(19) United States
(12) Patent Application Publication
(10) Pub. No.: US 2024/0245675 A1
(43) Pub. Date: Jul. 25, 2024

(54) PROTEIN SECRETION INHIBITORS

(71) Applicant: Gate Bioscience, Inc., Brisbane, CA (US)

(72) Inventors: Jordi MATA-FINK, Happy Valley, OR (US); Phillip Patrick SHARP, San Francisco, CA (US); Raman TALWAR, Denver, CO (US)

(21) Appl. No.: 18/425,076

(22) Filed: Jan. 29, 2024

Related U.S. Application Data
(63) Continuation of application No. PCT/US2022/074209, filed on Jul. 27, 2022.
(60) Provisional application No. 63/203,621, filed on Jul. 27, 2021.

(51) Int. Cl. A61K 31/496; A61K 31/426; A61K 31/427; A61K 31/437; A61K 31/4439; A61K 31/444; A61K 31/506; A61P 25/00; A61P 35/00; C07D 277/42; C07D 411/04; C07D 411/12; C07D 411/14; C07D 413/14; C07D 471/04

(52) U.S. Cl. CPC A61K 31/496; A61K 31/426; A61K 31/427; A61K 31/437; A61K 31/4439; A61K 31/444; A61K 31/506; A61P 25/00; A61P 35/00; C07D 277/42; C07D 411/04; C07D 411/12; C07D 411/14; C07D 413/14; C07D 471/04

(57) ABSTRACT

The present invention features compounds useful in the inhibition of the secretion of proteins. The compounds of the invention can be used in methods for treating diseases associated with protein secretion, and in methods for inhibiting protein secretion.

### SEQUENCE LISTING

The instant application contains a Sequence Listing which has been submitted electronically in XML format and is hereby incorporated by reference in its entirety. Said XML copy, created Jan. 26, 2024, is named ‘51573-003003_Sequence_Listing_1_26_24’ and is 4,594 bytes in size.

### FIELD OF THE INVENTION

In general, the present invention relates to small molecules that inhibit the secretion of proteins by inhibiting the Sec61 protein secretion complex. The invention further relates to methods for treating a subject having a disorder associated with the Sec61 protein secretion complex. The invention further relates to methods for inhibiting the translocation of a target protein through Sec61.

### BACKGROUND

Myriad proteins are secreted from cells, including proteins associated with disease. Sec61 is a membrane protein complex involved in the secretion of protein from cells, some of which are associated with disease. Sec61 is a component of the translocon, a complex of proteins which translocate proteins from the cytosol into the endoplasmic reticulum. Molecules capable of selectively inhibiting the secretion of specific, disease-associated proteins through Sec61, thereby preventing their secretion from cells, would be useful as therapeutic agents. Known inhibitors of protein secretion do not selectively inhibit the translocation of specific, disease-associated proteins through Sec61. Accordingly, there is a need for selective inhibitors of Sec61.

### SUMMARY OF THE INVENTION

In an aspect, this disclosure provides a compound, or a pharmaceutically acceptable salt thereof, having the structure of Formula 1:

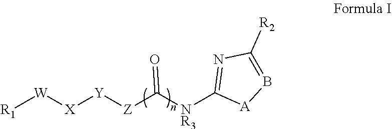

wherein
R₁ is H, optionally substituted C₁-C₆ alkyl, optionally substituted C₃-C₇ cycloalkyl, optionally substituted C₂-C₉ heterocycle, optionally substituted C₆-C₁₀ aryl, optionally substituted C₂-C₉ heteroaryl, C₂-C₉ heteroaryl C₁-C₆ alkyl, or Rₐ-R_{b}-R_{c},
   wherein Rₐ is optionally substituted C₆-C₁₀ arylene or optionally substituted C₂-C₉ heteroarylene;
   R_{b} is —O— or optionally substituted C₁-C₆ alkylene, and
   R_{c} is optionally substituted C₆-C₁₀ aryl;
Z is absent, optionally substituted C₃-C₁₀ cycloalkylene, optionally substituted C₂-C₉ heterocyclylene, C₆-C₁₀ arylene, or optionally substituted C₂-C₉ heteroarylene;
Y is absent, O, or NR₃;
X is absent or optionally substituted C₁-C₆ alkylene;
W is absent, S, O or NR₃;
n is 0 or 1;
A is S, O, NH, or CH;
B is C or N;
R₂ is optionally substituted C₁₋₆ alkyl, optionally substituted C₂-C₆ alkenyl, optionally substituted C₃₋₇ cycloalkyl, optionally substituted C₂₋₉ heterocycle, optionally substituted C₆-C₁₀ aryl, optionally substituted C₂₋₉ heteroaryl; optionally substituted C₂-C₉ heteroaryl C₁-C₆ alkyl, optionally substituted C₃-C₇ cycloalkyl C₁-C₆ aryl, optionally substituted C₁-C₆ heteroalkyl, cyano, or has the structure X₁—O—Y₁;
   wherein X₁ is optionally substituted C₁-C₆ alkylene, and
   Y₁ is optionally substituted C₁-C₆ alkyl, optionally substituted C₃-C₈ cycloalkyl, or optionally substituted C₆-C₁₀ aryl; and
each R₃ is, independently, H or C₁-C₆ alkyl.

In some embodiments, R₁ is H, optionally substituted C₁-C₆ alkyl, optionally substituted C₃-C₇ cycloalkyl, optionally substituted C₂-C₉ heterocycle, optionally substituted C₆-C₁₀ aryl, or optionally substituted C₂-C₉ heteroaryl;
Z is absent, optionally substituted C₃-C₁₀ cycloalkyl, optionally substituted C₂-C₉ heterocyclyl, C₆-C₁₀ aryl, or optionally substituted C₂-C₉ heteroaryl;
Y is absent, O, or NR₃;
X is absent, optionally substituted C₁-C₆ alkylene;
W is absent, S, O or NR₃
n is 0 or 1;
A is S, O, or CH;
B is C or N;
each R₃ is, independently, H or C₁-C₆ alkyl.

In some embodiments, n is 1.

In some embodiments, the compound of Formula I, or pharmaceutically acceptable salt thereof, has the structure of Formula II:

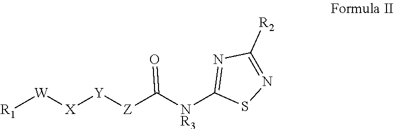

In some embodiments of the compound of Formula II, or the pharmaceutically acceptable salt thereof, Z is optionally substituted C₆-C₁₀ aryl, or optionally substituted C₂-C₉ heteroaryl. In some embodiments of the compound of Formula II, or the pharmaceutically acceptable salt thereof, Y is O or NR₃. In some embodiments of the compound of Formula II, or the pharmaceutically acceptable salt thereof, Y is O. In some embodiments of the compound of Formula II, or the pharmaceutically acceptable salt thereof, Y is NR₃. In some embodiments of the compound of Formula II, or the pharmaceutically acceptable salt thereof, Y is NH. In some embodiments of the compound of Formula II, or the pharmaceutically acceptable salt thereof, X is optionally substituted C₁-C₆ alkylene. In some embodiments of the compound of Formula II, or the pharmaceutically acceptable salt thereof, W is S, O, or NR₃. In some embodiments of the compound of Formula II, or the pharmaceutically acceptable salt thereof, W is S. In some embodiments of the compound of Formula II, or the pharmaceutically acceptable salt thereof, W is O. In some embodiments of the compound of Formula II, or the pharmaceutically acceptable salt thereof, W is NR₃. In some embodiments of the compound of Formula II, or the pharmaceutically acceptable salt thereof, W is NH.

In some embodiments, the compound of Formula I, or the pharmaceutically acceptable salt thereof, has the structure of Formula III:

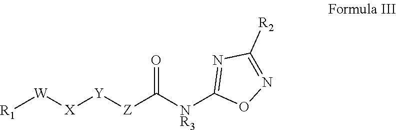

In some embodiments of the compound of Formula III, or the pharmaceutically acceptable salt thereof, Z is optionally substituted C₆-C₁₀ aryl, or optionally substituted C₂-C₉ heteroaryl. In some embodiments of the compound of Formula III, or the pharmaceutically acceptable salt thereof, Y is O or NR₃. In some embodiments of the compound of Formula III, or the pharmaceutically acceptable salt thereof, Y is O. In some embodiments of the compound of Formula III, or the pharmaceutically acceptable salt thereof, Y is NR₃. In some embodiments of the compound of Formula III, or the pharmaceutically acceptable salt thereof, Y is NH. In some embodiments of the compound of Formula III, or the pharmaceutically acceptable salt thereof, X is optionally substituted C₁-C₆ alkylene. In some embodiments of the compound of Formula III, or the pharmaceutically acceptable salt thereof, W is S, O, or NR₃. In some embodiments of the compound of Formula III, or the pharmaceutically acceptable salt thereof, W is S. In some embodiments of the compound of Formula III, or the pharmaceutically acceptable salt thereof, W is O. In some embodiments of the compound of Formula III, or the pharmaceutically acceptable salt thereof, W is NR₃. In some embodiments of the compound of Formula III, or the pharmaceutically acceptable salt thereof, W is NH.

In some embodiments, the compound of Formula I, or the pharmaceutically acceptable salt thereof, has the structure of Formula IV:

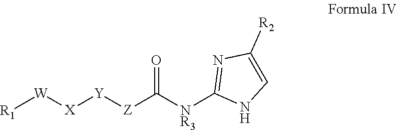

In some embodiments of the compound of Formula IV, or the pharmaceutically acceptable salt thereof, Z is optionally substituted C₆-C₁₀ aryl, or optionally substituted C₂-C₉ heteroaryl. In some embodiments of the compound of Formula IV, or the pharmaceutically acceptable salt thereof, Y is O or NR₃. In some embodiments of the compound of Formula IV, or the pharmaceutically acceptable salt thereof, Y is O. In some embodiments of the compound of Formula IV, or the pharmaceutically acceptable salt thereof, Y is NR₃. In some embodiments of the compound of Formula IV, or the pharmaceutically acceptable salt thereof, Y is NH. In some embodiments of the compound of Formula IV, or the pharmaceutically acceptable salt thereof, X is optionally substituted C₁-C₆ alkylene. In some embodiments of the compound of Formula IV, or the pharmaceutically acceptable salt thereof, W is S, O, or NR₃. In some embodiments of the compound of Formula IV, or the pharmaceutically acceptable salt thereof, W is S. In some embodiments of the compound of Formula IV, or the pharmaceutically acceptable salt thereof, W is O. In some embodiments of the compound of Formula IV, or the pharmaceutically acceptable salt thereof, W is NR₃. In some embodiments of the compound of Formula IV, or the pharmaceutically acceptable salt thereof, W is NH.

In some embodiments, the compound of Formula I, or the pharmaceutically acceptable salt thereof, A is S and B is C.

In some embodiments, the compound of Formula I, or the pharmaceutically acceptable salt thereof, has the structure of Formula V:

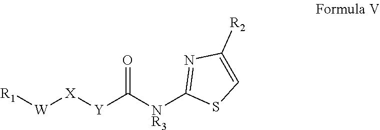

wherein Y is O or NR₃.

In some embodiments of the compound of Formula V, or the pharmaceutically acceptable salt thereof, X is an optionally substituted C₁-C₆ alkylene. In some embodiments of the compound of Formula V, or the pharmaceutically acceptable salt thereof, Y is O. In some embodiments of the compound of Formula V, or the pharmaceutically acceptable salt thereof, Y is NR₃. In some embodiments of the compound of Formula V, or the pharmaceutically acceptable salt thereof, Y is NH.

In some embodiments, the compound of Formula I, or the pharmaceutically acceptable salt thereof, has the structure of Formula VI:

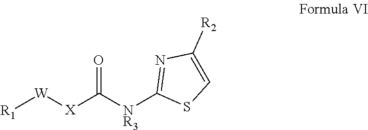

wherein W is NR₃

In some embodiments of the compound of Formula VI, or the pharmaceutically acceptable salt thereof, X is optionally substituted C₁-C₆ alkylene. In some embodiments of the compound of Formula VI, or the pharmaceutically acceptable salt thereof, W is NH.

In some embodiments of the compound of Formula I, or the pharmaceutically acceptable salt thereof, n is 0.

In some embodiments of the compound of any one of Formulae I-IV, or the pharmaceutically acceptable salt thereof, Z is an optionally substituted phenylene, optionally substituted pyridinylene, optionally substituted pyrimidinylene, optionally substituted pyridazinylene, pyrazinylene, triazinylene, optionally substituted tetrazinylene, optionally substituted thiophenylene, optionally substituted pyrrolylene, optionally substituted furanylene, optionally substituted pyrazolylene, optionally substituted thiazolylene, optionally substituted oxadiazolylene, optionally substituted thiadiazolylene, optionally substituted isoxazolylene, optionally substituted isothiazolylene, optionally substituted thiazolylene, optionally substituted oxazolylene, optionally substituted imidazolylene, optionally substituted cyclohexylene, optionally substituted cyclopentylene, optionally substituted cyclobutylene, optionally substituted cyclopropylene, optionally substituted cycloheptylene, optionally substituted cyclooctylene, optionally substituted indolylene, or optionally substituted azaindolylene. In some embodiments, Z is substituted with at least one halogen, e.g., at least one fluorine.

In some embodiments, Z has the structure Formula VIIi:

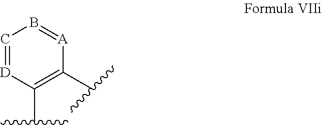

wherein A, B, C and D are, each, independently, N, CH, or CR₄; and
each R₄ is independently optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol.

In some embodiments, Z has the structure of Formula VIIIi:

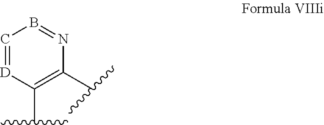

wherein B, C, and D are each, independently, CH or CR₄.

In some embodiments, Z has the structure of Formula IXi:

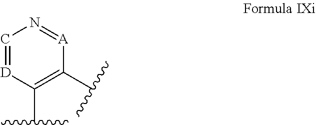

wherein A, C, and D are each, independently, CH or CR₄.

In some embodiments, Z has the structure of Formula Xi:

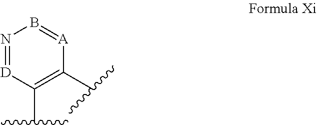

wherein A, B, and D are each, independently, CH or CR₄.

In some embodiments, Z has the structure of Formula XIi:

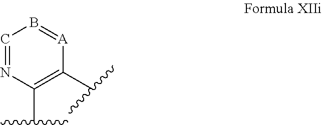

wherein A, B, and C are each, independently, CH or CR₄.

In some embodiments, Z has the structure of Formula XIIi:

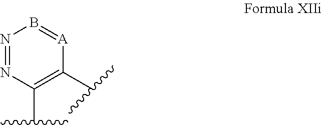

wherein A and B are each, independently, CH or CR₄.

In some embodiments, Z has the structure of Formula XIIIi:

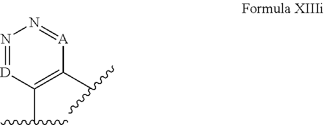

wherein A and D are each, independently, CH or CR₄.

In some embodiments, Z has the structure of Formula XIVi:

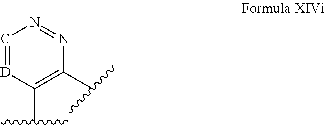

wherein C and D are each, independently, CH or CR₄.

In some embodiments, Z has the structure of Formula XVi:

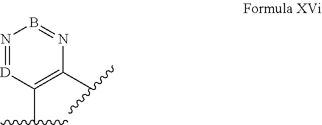

wherein B and D are each, independently, CH or CR₄.

In some embodiments, Z has the structure of Formula XVIi:

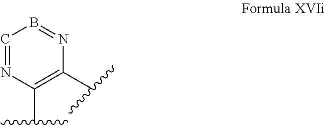

wherein B and C are each, independently, CH or CR₄.

In some embodiments, Z has the structure of Formula XVIIi:

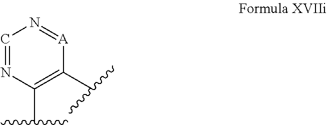

wherein A and C are each, independently, CH or CR₄.

In some embodiments, Z has the structure of any one of Formula XVIIIi:

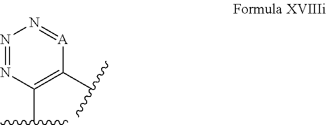

wherein A is CH or CR₄.

In some embodiments, Z has the structure of Formula XIXi:

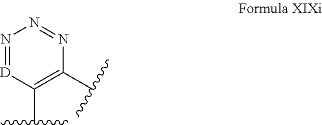

wherein D Is CH or CR₄

In some embodiments, Z has the structure of Formula XXi:

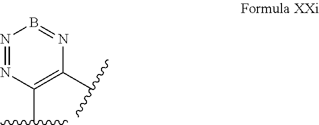

wherein B is CH or CR₄.

In some embodiments, Z has the structure of Formula XXIi:

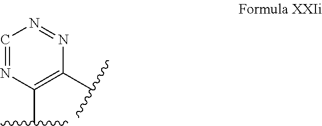

wherein C is CH or CR₄.

In some embodiments, Z has the structure of Formula XXIIi:

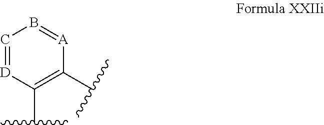

wherein A, B, C, and D are each, independently, CH or CR₄

In some embodiments, Z has the structure of Formula XXIIIi:

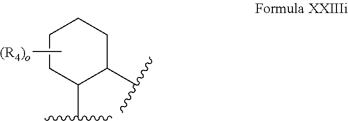

wherein o is 0, 1, 2, 3, or 4; and
each R₄ is, independently, optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol.

In some embodiments, Z has the structure of Formula XXIVi:

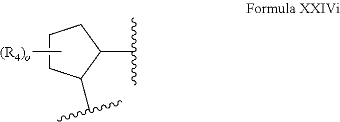

wherein o is 0, 1, 2, or 3; and
each R₄ is, independently, optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol.

In some embodiments, Z has the structure of Formula XXVi:

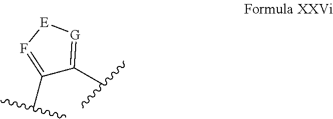

wherein
F and G are each, independently, N, CH, or CR₄;
each R₄ is independently optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol; and E is NH, O, or S.
In some embodiments, Z has the structure of Formula XXVIi:

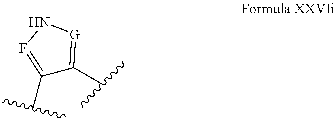

wherein F and G are each, independently, CH or CR₄.

In some embodiments, Z has the structure of Formula XXVIIi:

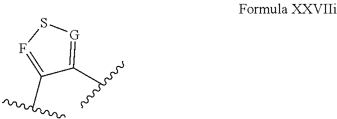

wherein F and G are each, independently, CH or CR₄.

In some embodiments, Z has the structure of Formula XXVIIIi:

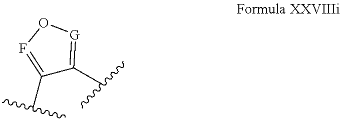

wherein F and G are each, independently, CH or CR₄.

In some embodiments, Z has the structure of Formula XXIXi:

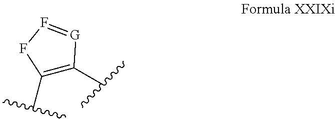

wherein
F and G are each, independently, N, CH, or CR₄;
each R₄ is independently optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol; and
E is NH, O, or S.

In some embodiments, Z has the structure of Formula XXXi:

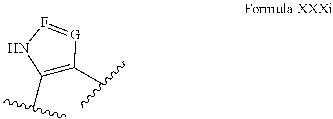

wherein F and G are each, independently, CH or CR₄.

In some embodiments, Z has the structure of Formula XXXIi:

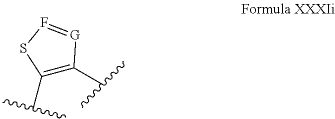

wherein F and G are each, independently, CH or CR₄.

In some embodiments, Z has the structure of Formula XXXIIi:

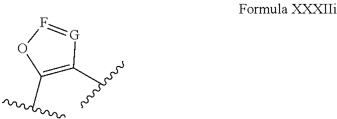

wherein F and G are each, independently, CH or CR₄.

In some embodiments, Z has the structure of Formula XXXIIIi:

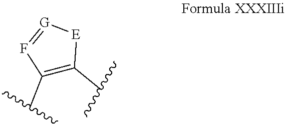

wherein
E is NH, O, or S;
F and G are each, independently, N, CH, or CR₄; and
   each R₄ is independently optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol.

In some embodiments, Z has the structure of Formula XXXIVi:

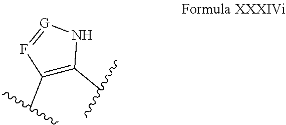

wherein F and G are each, independently, CH or CR₄.

In some embodiments, Z has the structure of Formula XXXVi:

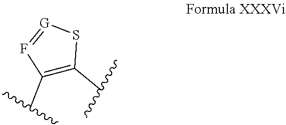

wherein F and G are each, independently, CH or CR₄.

In some embodiments, Z has the structure of Formula XXXVIi:

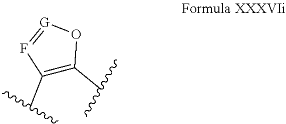

wherein F and G are each, independently, CH or CR₄.

In some embodiments, Z has the structure of Formula XXXVIIi:

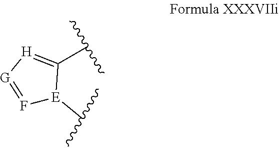

wherein E is N or CH;
F, G, and H are each, independently, N, CH, or CR₄; and
   each R₄ is independently optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol.

In some embodiments, Z has the structure of Formula XXXVIIIi:

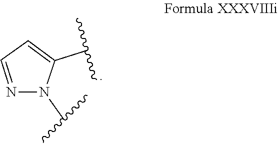 [SMILES: *c1ccnn1*]

In some embodiments, Z has the structure of Formula XXXIXi:

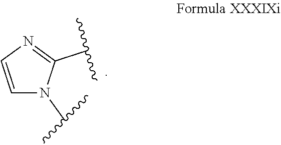 [SMILES: *c1nccn1*]

In some embodiments, Z has the structure of Formula XXXXi:

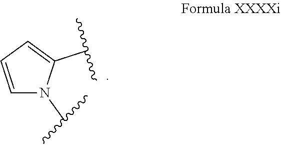 [SMILES: *c1cccn1*]

In some embodiments, Z has the structure of Formula XXXXIi:

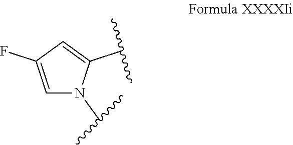 [SMILES: *c1cc(F)cn1*]

In some embodiments, Z has the structure of Formula XXXXIVi:

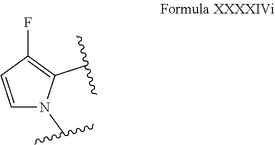 [SMILES: *c1c(F)ccn1*]

In some embodiments, Z has the structure of Formula XXXXIIi:

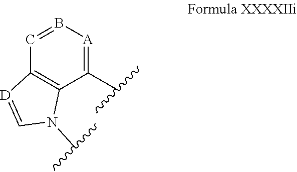

wherein A, B, C, and D are each, independently, CH, CR₄, or N.

In some embodiments, Z has the structure of Formula XXXXIIIi:

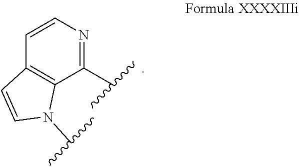 [SMILES: *c1nccc2ccn(*)c12]

In some embodiments, Z has the structure of Formula XXXXVi:

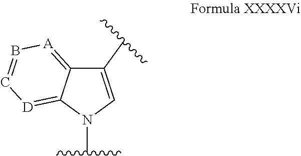

wherein A, B, C, and D are each, independently, CH, CR₄, or N; and
each R₄ is independently optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol.

In some embodiments, the compound of Formula I, or the pharmaceutically acceptable salt thereof, has the structure of Formula VII:

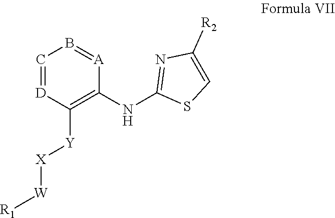

wherein A, B, C and D are, independently, N, CH, or CR₄; and
R₄ is optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol.

In some embodiments, the compound of Formula VII, or the pharmaceutically acceptable salt thereof, has the structure of Formula VIII:

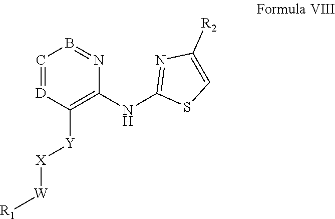

wherein B, C, and D are each, independently, CH or CR₄.

In some embodiments, the compound of Formula VII, or the pharmaceutically acceptable salt thereof, has the structure of Formula IX:

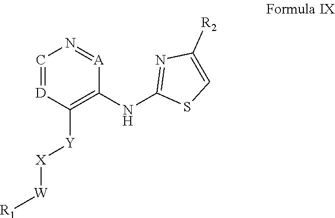

wherein A, C, and D are each, independently, CH or CR₄.

In some embodiments, the compound of Formula VII, or the pharmaceutically acceptable salt thereof, has the structure of Formula X:

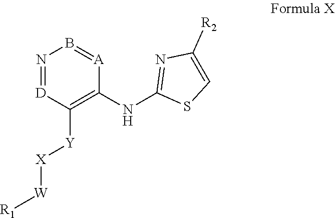

wherein A, B, and D are each, independently, CH or CR₄.

In some embodiments, the compound of Formula VII, or the pharmaceutically acceptable salt thereof, has the structure of Formula XI:

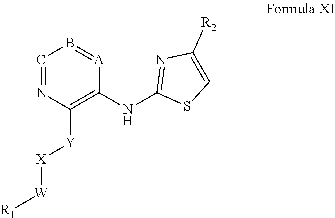

wherein A, B, and C are each, independently, CH or CR₄.

In some embodiments, the compound of Formula VII, or the pharmaceutically acceptable salt thereof, has the structure of Formula XII:

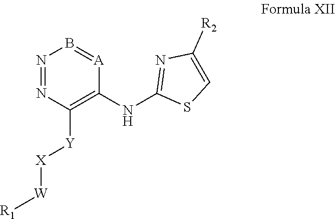

wherein A and B are each, independently, CH or CR₄.

In some embodiments, the compound of Formula VII, or the pharmaceutically acceptable salt thereof, has the structure of Formula XIII:

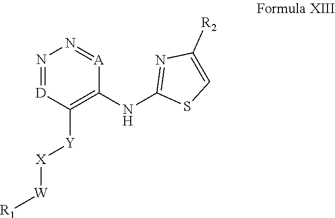

wherein A and D are each, independently, CH or CR₄.

In some embodiments, the compound of Formula VII, or the pharmaceutically acceptable salt thereof, has the structure of Formula XIII:

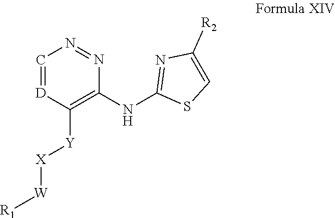

wherein C and D are each, independently, CH or CR₄.

In some embodiments, the compound of Formula VII, or the pharmaceutically acceptable salt thereof, has the structure of Formula XV:

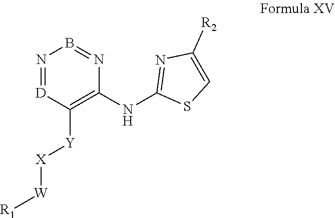

wherein B and D are each, independently, CH or CR₄.

In some embodiments, the compound of Formula VII, or the pharmaceutically acceptable salt thereof, has the structure of Formula XVI:

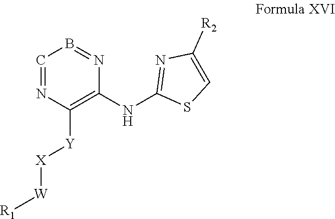

wherein B and C are each, independently, CH or CR₄.

In some embodiments, the compound of Formula VII, or the pharmaceutically acceptable salt thereof, has the structure of Formula XVII:

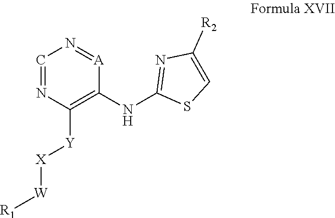

wherein A and C are each, independently, CH or CR₄.

In some embodiments, the compound of Formula VII, or the pharmaceutically acceptable salt thereof, has the structure of Formula XVIII:

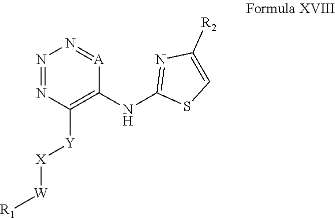

wherein A is CH or CR₄.

In some embodiments, the compound of Formula VII, or the pharmaceutically acceptable salt thereof, has the structure of Formula XIX:

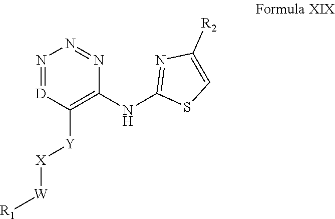

wherein D Is CH or CR₄

In some embodiments, the compound of Formula VII, or the pharmaceutically acceptable salt thereof, has the structure of Formula XX:

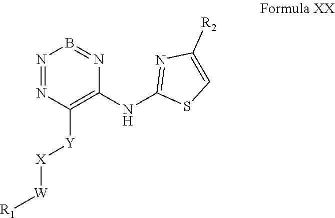

wherein B is CH or CR₄.

In some embodiments, the compound of Formula VII, or the pharmaceutically acceptable salt thereof, has the structure of Formula XXI:

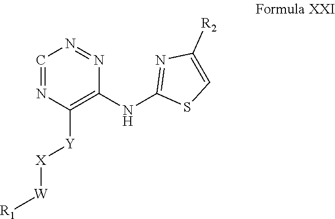

wherein C is CH or CR₄.

In some embodiments, the compound of Formula VIII, or the pharmaceutically acceptable salt thereof, has the structure of Formula XXII:

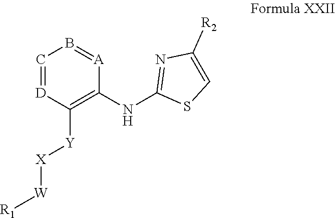

wherein A, B, C, and D are each, independently, CH or CR₄.

In some embodiments, the compound of Formula I, or the pharmaceutically acceptable salt thereof, has the structure of Formula XXIII:

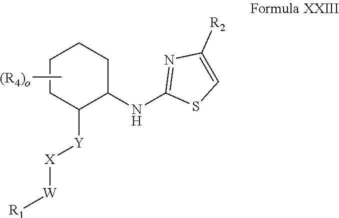

wherein o is 0, 1, 2, 3, or 4; and

each R₄ is, independently, optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol.

In some embodiments, the compound of Formula I, or the pharmaceutically acceptable salt thereof,

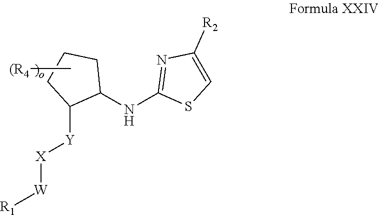

wherein o is 0, 1, 2, or 3; and
each R₄ is, independently, optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol.

In some embodiments, the compound of Formula I, or the pharmaceutically acceptable salt thereof, has the structure of Formula XXV:

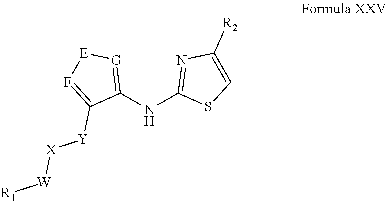

wherein E is NH, O, or S;
F and G are each, independently, N, CH, or CR₄; and
each R₄ is independently optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol.

In some embodiments, the compound of Formula XXV, or the pharmaceutically acceptable salt thereof, has the structure of Formula XXVI:

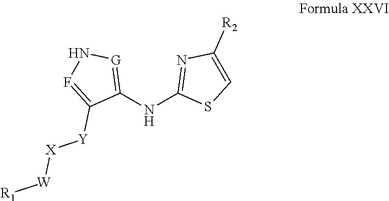

wherein F and G are each, independently, CH or CR₄.

In some embodiments, the compound of Formula XXV, or the pharmaceutically acceptable salt thereof, has the structure of Formula XXVII:

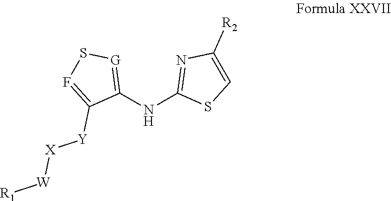

wherein F and G are each, independently, CH or CR₄

In some embodiments, the compound of Formula XXV, or the pharmaceutically acceptable salt thereof, has the structure of Formula XXVIII:

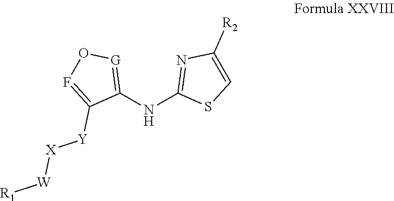

wherein F and G are each, independently, CH or CR₄.

In some embodiments, the compound of Formula I, or the pharmaceutically acceptable salt thereof, has the structure of Formula XXIX:

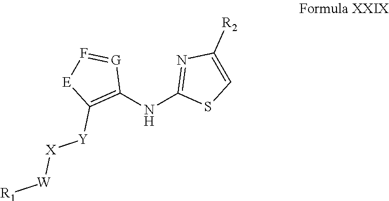

wherein E is NH, O, or S;
F and G are each, independently, N, CH, or CR₄; and
each R₄ is independently optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol.

In some embodiments, the compound of Formula XXIX, or the pharmaceutically acceptable salt thereof, has the structure of Formula XXX:

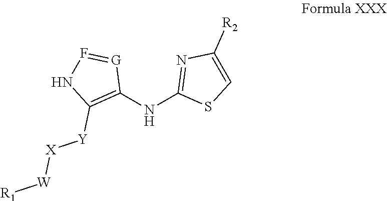

wherein F and G are each, independently, CH or CR₄.

In some embodiments, the compound of Formula XXIX, or the pharmaceutically acceptable salt thereof, has the structure of Formula XXXI:

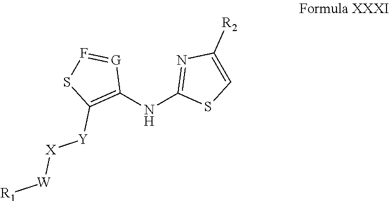

wherein F and G are each, independently, CH or CR₄.

In some embodiments, the compound of Formula XXIX, or the pharmaceutically acceptable salt thereof, has the structure of Formula XXXII:

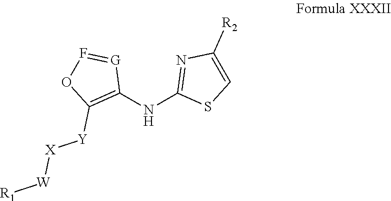

wherein F and G are each, independently, CH or CR₄.

In some embodiments, the compound of Formula I, or the pharmaceutically acceptable salt thereof, has the structure of Formula XXXIII:

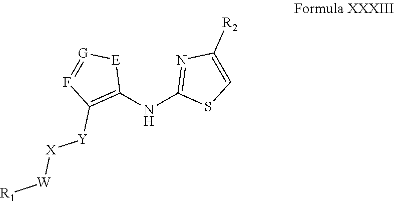

wherein E is NH, O, or S;
F and G are each, independently, N, CH, or CR₄; and
each R₄ is independently optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol;

In some embodiments, the compound of Formula XXXIII, or the pharmaceutically acceptable salt thereof, has the structure of Formula XXXIV:

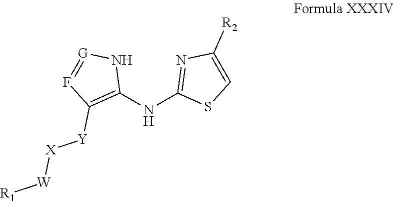

wherein F and G are each, independently, CH or CR₄.

In some embodiments, the compound of Formula XXXIII, or the pharmaceutically acceptable salt thereof, has the structure of Formula XXXV:

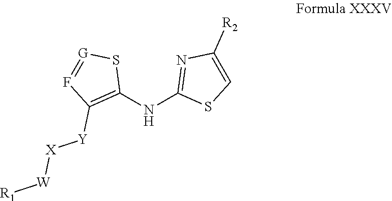

wherein F and G are each, independently, CH or CR₄.

In some embodiments, the compound of Formula XXXIII, or the pharmaceutically acceptable salt thereof, has the structure of Formula XXXVI:

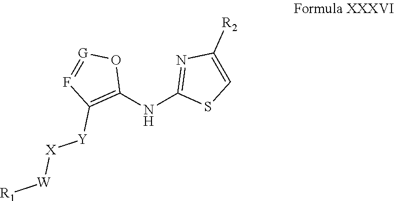

wherein F and G are each, independently, CH or CR₄.

In some embodiments, the compound of Formula I, or the pharmaceutically acceptable salt thereof has the structure of Formula XXXVII:

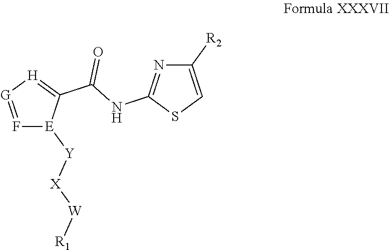

wherein E is N or CH;
F, G, and H are each, independently, N, CH, or CR₄; and
each R₄ is independently optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol; In some embodiments, the compound, or pharmaceutically acceptable salt thereof, of formula XXXVII has the structure of Formula XXXVIII:

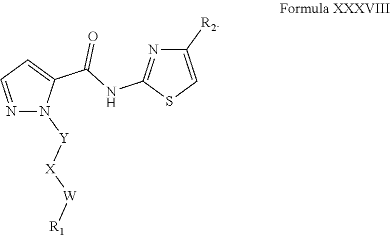

In some embodiments, the compound of Formula XXXVI, or the pharmaceutically acceptable salt thereof has the structure of Formula XXXIX:

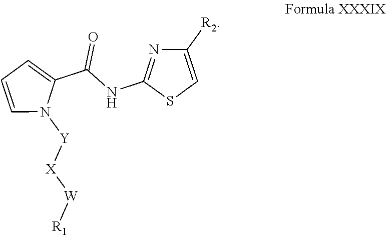

In some embodiments, the compound of Formula XXXVII, or the pharmaceutically acceptable salt thereof has the structure of Formula XXXX:

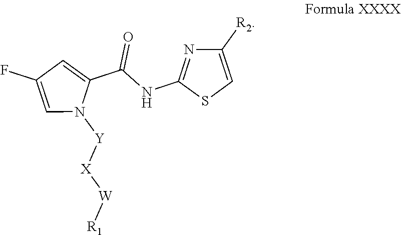

In some embodiments, the compound of Formula XXXVII, or the pharmaceutically acceptable salt thereof, has the structure of Formula XXXXIii:

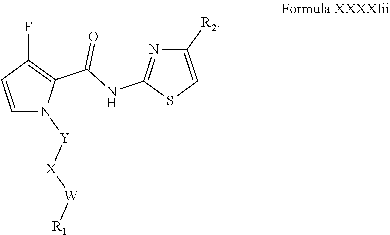

In some embodiments, the compound of Formula I, or the pharmaceutically acceptable salt thereof, has the structure of Formula XXXXII:

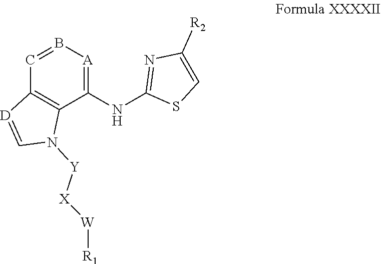

wherein A, B, C, and D are each, independently, CH, CR₄, or N; and
each R is independently optionally substituted aryl, optionally carbocyclyl, halogen, hydroxyl, optionally substituted heteroalkyl, optionally substituted heteroaryl, optionally substituted heterocyclyl, optionally substituted amino azido, cyano, nitro, or thiol.

In some embodiments, the compound of Formula XXXXII, or the pharmaceutically acceptable salt thereof, has the structure of Formula XXXXIII:

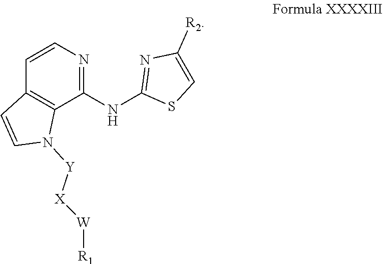

In some embodiments of the compound of any one of Formulae VII-XXXXIII, or the pharmaceutically acceptable salt thereof, X is C₁-C₆ alkylene. In some embodiments of the compound of any one of Formulae VII-XXXXIII, or the pharmaceutically acceptable salt thereof, Y is O or NR₃. In some embodiments of the compound of any one of Formulae VII-XXXXIII, or the pharmaceutically acceptable salt thereof, Y is O. In some embodiments of the compound of any one of Formulae VII-XXXXIII, Y is NR₃. In some embodiments of the compound of any one of Formulae VII-XXXXIII, Y is NH. In some embodiments of the compound of any one of Formulae VII-XXXXIII, or the pharmaceutically acceptable salt thereof, W is S, O, or NR₃. In some embodiments of the compound of any one of Formulae VII-XXXXIII, or the pharmaceutically acceptable salt thereof, W is S. In some embodiments of the compound of any one of Formulae VII-XXXXIII, or the pharmaceutically acceptable salt thereof, W is O. In some embodiments of the compound of any one of Formulae VII-XXXXIII, or the pharmaceutically acceptable salt thereof, W is NR₃. In some embodiments of the compound of any one of Formulae VII-XXXXIII, or the pharmaceutically acceptable salt thereof, W is S, O, or NH.

In some embodiments of the compound of any one of Formulae I-XXXXIII, or the pharmaceutically acceptable salt thereof, R₂ is an optionally substituted C₁-C₆ alkyl, e.g., methyl, ethyl, propyl, isopropyl, butyl, tert-butyl, pentyl, neopentyl, or hexyl. In some embodiments, R₂ is

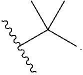 [SMILES: *C(C)(C)C]

In some embodiments of the compound of any one of Formulae I-XXXXIII, or the pharmaceutically acceptable salt thereof, R₂ is an optionally substituted C₂-C₉ heterocyclyl. In some embodiments, R₂ is an optionally substituted pyrrolidinyl group. In some embodiments, R₂ is substituted with one or more fluorine, e.g., one fluorine substituent, two fluorine substituents, or three fluorine substituents. In some embodiments, R₂

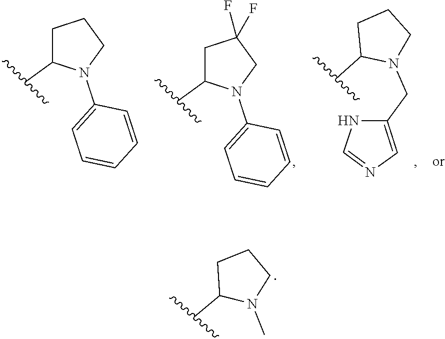

In some embodiments of the compound of any one of Formulae I-XXXXIII, or the pharmaceutically acceptable salt thereof, R₂ is an optionally substituted C₂-C₉ heteroaryl, e.g., an optionally substituted pyridine or optionally substituted oxazole. In some embodiments, R₂ is an optionally substituted pyridinyl group. In some embodiments, R₂ is

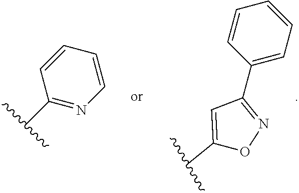 [SMILES: *c1ccccn1 ; *c1cc(-c2ccccc2)no1]

In some embodiments of the compound of any one of Formulae I-XXXXIII, or the pharmaceutically acceptable salt thereof, R₂ is an optionally substituted C₁-C₆ heteroalkyl, e.g., optionally substituted methoxy, ethoxy, propoxy, or butoxy.

In some embodiments of the compound of any one of Formulae I-XXXXIII, R₂ has the structure X₁—O—Y₁. In some embodiments, X₁ is substituted with at least one methyl group, e.g., one methyl group, two methyl groups, or three methyl groups. In some embodiments, Y₁ is an optionally substituted phenyl group.

In some embodiments, Y₁ is an optionally substituted cyclopropyl group. In some embodiments, R₂ is

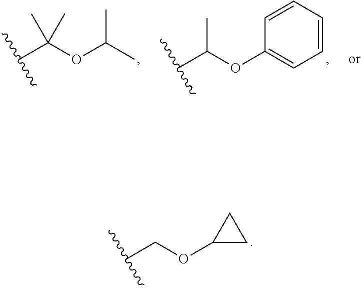

In some embodiments of the compound of any one of Formulae I-XXXXIII, or the pharmaceutically acceptable salt thereof, R₂ is an optionally substituted C₆-C₁₀ aryl. In some embodiments, R₂ is phenyl,

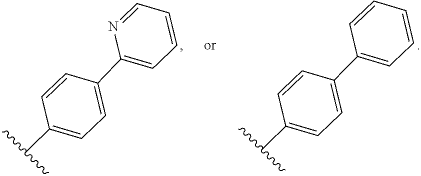 [SMILES: *c1ccc(-c2ccccn2)cc1 ; *c1ccc(-c2ccccc2)cc1]

In some embodiments of the compound of any one of Formulae I-XXXXIII, R² is optionally substituted C₂-C₆ alkenyl. In some embodiments, R² is

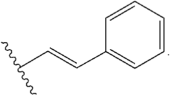 [SMILES: */C=C/c1ccccc1]

In some embodiments of the compound of any one of Formulae I-XXXXIII, or the pharmaceutically acceptable salt thereof, R₁ is an optionally substituted C₂-C₉ heteroaryl, e.g., an optionally substituted pyridinyl or optionally substituted oxazolyl. In some embodiments, R₁ is

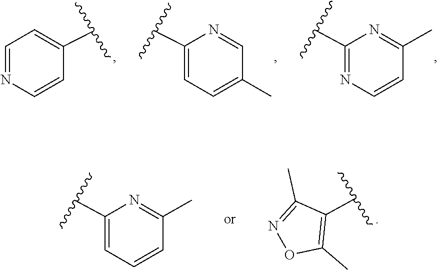

In some embodiments of the compound of any one of Formulae I-XXXXIII, or the pharmaceutically acceptable salt thereof, R₁ is Rₐ-R_{b}-R_{c}. In some embodiments, Rₐ is an optionally substituted C₆-C₁₀ arylene. In some embodiments, Rₐ is an optionally substituted C₂-C₉ heteroarylene. In some embodiments, R_{b} is —O—.

In some embodiments, R₁ is.

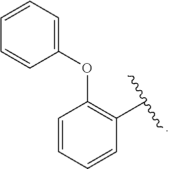 [SMILES: *c1ccccc1Oc1ccccc1]

In some embodiments, R_{b} is an optionally substituted C₁-C₆ alkylene. In some embodiments, R₁ is

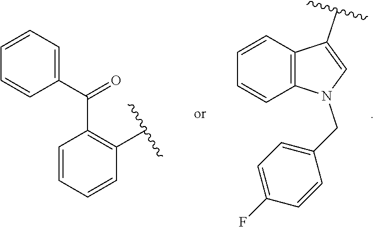 [SMILES: *c1ccccc1C(=O)c1ccccc1 ; *c1cn(Cc2ccc(F)cc2)c2ccccc12]

In some embodiments of the compound of any one of Formulae I-XXXXIII, or the pharmaceutically acceptable salt thereof, R₁ is an optionally substituted C₂-C₉ heteroaryl C₁-C₆ alkyl. In some embodiments, R₁ is

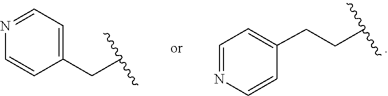 [SMILES: *Cc1ccncc1 ; *CCc1ccncc1]

In some embodiments of the compound of any one of Formulae I-XXXXIII, or the pharmaceutically acceptable salt thereof, R₁ is an optionally substituted C₆-C₁₀ aryl, e.g., an optionally substituted phenyl. In some embodiments, R₁ is

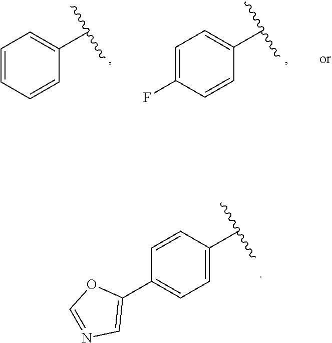 [SMILES: *c1ccccc1 ; *c1ccc(F)cc1 ; *c1ccc(-c2cnco2)cc1]

In some embodiments of the compound of any one of Formulae I-XXXXIII, or the pharmaceutically acceptable salt thereof, X is

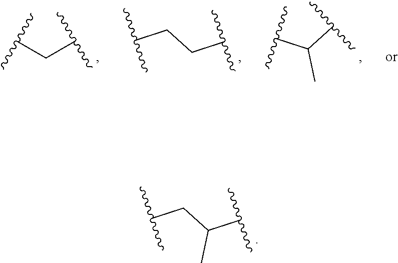

In some embodiments, X is

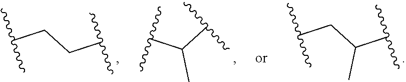

In some embodiments, the compound of any one of Formulae I-XXXXIII has the structure 144.

In some embodiments, the compound has the structure of

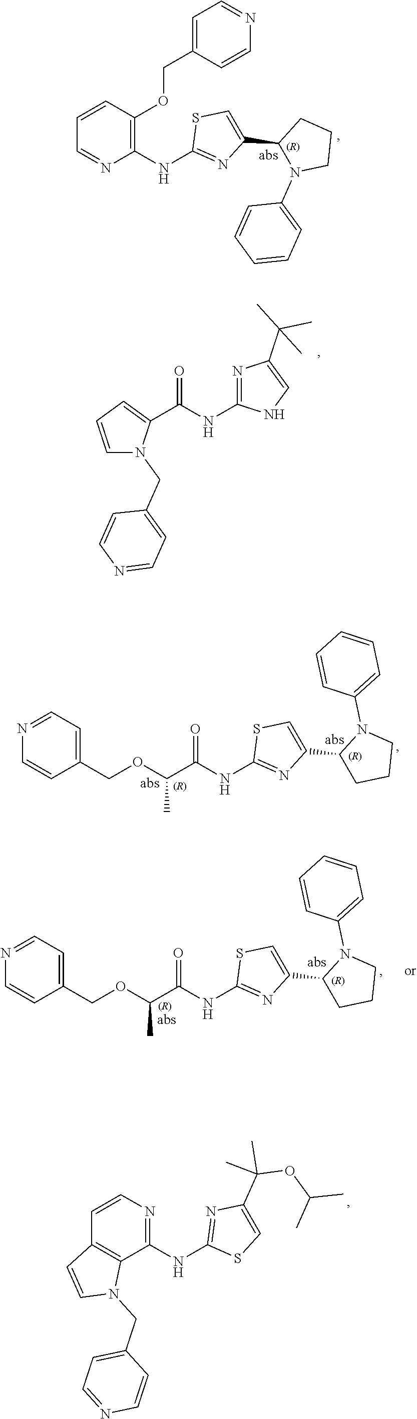

or a pharmaceutically acceptable salt thereof.

In some embodiments, the compound of any one of Formulae I-XXXXIII has the structure of any one of compounds 1-50 in Table 1.

In another aspect, the disclosure provides a pharmaceutical composition comprising any of the foregoing compounds, or pharmaceutically acceptable salts thereof, and a pharmaceutically acceptable excipient.

In another aspect, the disclosure provides a method of treating a Sec61-associated disorder in a subject in need thereof, the method comprising administering, or contacting the cell with, an effective amount of any of the foregoing compounds, or pharmaceutically acceptable salts thereof, or a pharmaceutical composition comprising any of the foregoing compounds, or pharmaceutically acceptable salts thereof, and a pharmaceutically acceptable excipient.

Sec61-associated diseases or disorders include, but are not limited to, amyloidosis, light chain amyloidosis, autoantibody diseases, chronic kidney disease, fibrosis, neurodegeneration, autoimmune disease, genetically-defined kidney disease, viral disease, influenza, dengue virus, zika virus, hepatitis B virus, hepatitis C virus, SARS-CoV-2, human immunodeficiency virus, malaria, cancer, glioma, myeloma, multiple types of cancer with solid tumors, autoimmune diseases, rheumatoid arthritis, ankylosing spondylitis, celiac disease, multiple sclerosis, atopic dermatitis, Crohn's disease, psoriasis, allergic asthma, autoimmune antibody diseases, myasthenia gravis, neuromyelitis optica, warm antibody hemolytic anemia, prion disease, immune thrombocytopenic purpura, chronic inflammatory demyelinating polyradiculoneuropathy, fibrotic diseases, idiopathic pulmonary fibrosis, endometriosis, nonalcoholic steatohepatitis, neurodegenerative diseases, Alzheimer's disease, Parkinson's disease, Huntington's disease, amyotrophic lateral sclerosis, hypercholesterolemia, Creutzfeldt-Jakob disease, Gerstmann-Sträussler-Scheinker syndrome, high cholesterol, metabolic syndrome, and fatal familial insomnia.

In some embodiments, the disclosure provides a method of treating viral diseases, cancer, prion disease, light chain amyloidosis, autoimmune antibody disease, genetically-defined kidney disease, or malaria, the method comprising administering, or contacting the cell with, an effective amount of any of the foregoing compounds, or pharmaceutically acceptable salts thereof, or a pharmaceutical composition comprising any of the foregoing compounds, or pharmaceutically acceptable salts thereof, and a pharmaceutically acceptable excipient. In another aspect, the disclosure provides a method of inhibiting the translocation of a target protein through Sec-61, the method comprising contacting a cell with an effective amount of any of the foregoing compounds, or pharmaceutically acceptable salts thereof, or a pharmaceutical composition comprising any of the foregoing compounds, or pharmaceutically acceptable salts thereof, and a pharmaceutically acceptable excipient. In some embodiments, the inhibition of translocation is selective for a target protein over a non-target protein.

### Chemical Terms

It is to be understood that the terminology employed herein is for the purpose of describing particular embodiments and is not intended to be limiting.

The term “acyl,” as used herein, represents a hydrogen or an alkyl group, as defined herein, that is attached to a parent molecular group through a carbonyl group, as defined herein, and is exemplified by formyl (i.e., a carboxyaldehyde group), acetyl, trifluoroacetyl, propionyl, and butanoyl. Exemplary unsubstituted acyl groups include from 1 to 6, from 1 to 11, or from 1 to 21 carbons.

The term “alkyl,” as used herein, refers to a branched or straight-chain monovalent saturated aliphatic hydrocarbon radical of 1 to 20 carbon atoms (e.g., 1 to 16 carbon atoms, 1 to 10 carbon atoms, or 1 to 6 carbon atoms). An alkylene is a divalent alkyl group.

The term “alkenyl,” as used herein, alone or in combination with other groups, refers to a straight-chain or branched hydrocarbon residue having a carbon-carbon double bond and having 2 to 20 carbon atoms (e.g., 2 to 16 carbon atoms, 2 to 10 carbon atoms, 2 to 6, or 2 carbon atoms).

The term “alkynyl,” as used herein, alone or in combination with other groups, refers to a straight-chain or branched hydrocarbon residue having a carbon-carbon triple bond and having 2 to 20 carbon atoms (e.g., 2 to 16 carbon atoms, 2 to 10 carbon atoms, 2 to 6, or 2 carbon atoms).

The term “amino,” as used herein, represents —N(R^{N1})₂, wherein each R^{N1} is, independently, H, OH, NO₂, N(R^{N2})₂, SO₂OR^{N2}, SO₂R^{N2}, SOR^{N2}, an N-protecting group, alkyl, alkoxy, aryl, arylalkyl, cycloalkyl, acyl (e.g., acetyl, trifluoroacetyl, or others described herein), wherein each of these recited R^{N1} groups can be optionally substituted; or two R^{N1} combine to form an alkylene or heteroalkylene, and wherein each R^{N2} is, independently, H, alkyl, or aryl. The amino groups of the invention can be an unsubstituted amino (i.e., —NH₂) or a substituted amino (i.e., —N(R^{N})₂).

The term “aryl,” as used herein, refers to an aromatic mono- or polycarbocyclic radical of 6 to 12 carbon atoms having at least one aromatic ring. Examples of such groups include, but are not limited to, phenyl, naphthyl, 1,2,3,4-tetrahydronaphthyl, 1,2-dihydronaphthyl, indanyl, and 1H-indenyl.

The term “arylalkyl,” as used herein, represents an alkyl group substituted with an aryl group. Exemplary unsubstituted arylalkyl groups are from 7 to 30 carbons (e.g., from 7 to 16 or from 7 to 20 carbons, such as C₆₋₁₀ aryl C₁₋₆ alkyl, C₆₋₁₀ aryl C₁₋₁₀alkyl, or C₆₋₁₀ aryl C₁₋₂₀ alkyl), such as, benzyl and phenethyl. In some embodiments, the alkyl and the aryl each can be further substituted with 1, 2, 3, or 4 substituent groups as defined herein for the respective groups.

The term “azido,” as used herein, represents a —N₃ group.

The term “cyano,” as used herein, represents a —CN group.

The terms “carbocyclyl,” as used herein, refer to a non-aromatic C₃-12 monocyclic, bicyclic, or tricyclic structure in which the rings are formed by carbon atoms. Carbocyclyl structures include cycloalkyl groups and unsaturated carbocyclyl radicals.

The term “cycloalkyl,” as used herein, refers to a saturated, non-aromatic, monovalent mono- or polycarbocyclic radical of three to ten, preferably three to six carbon atoms. This term is further exemplified by radicals such as cyclopropyl, cyclobutyl, cyclopentyl, cyclohexyl, cycloheptyl, norbornyl, and adamantyl.

The term “halogen,” as used herein, means a fluorine (fluoro), chlorine (chloro), bromine (bromo), or iodine (iodo) radical.

The term “heteroalkyl,” as used herein, refers to an alkyl group, as defined herein, in which one or more of the constituent carbon atoms have been replaced by nitrogen, oxygen, or sulfur. In some embodiments, the heteroalkyl group can be further substituted with 1, 2, 3, or 4 substituent groups as described herein for alkyl groups. Examples of heteroalkyl groups are an “alkoxy” which, as used herein, refers alkyl-O— (e.g., methoxy and ethoxy). A heteroalkylene is a divalent heteroalkyl group.

The term “heteroalkenyl,” as used herein, refers to an alkenyl group, as defined herein, in which one or more of the constituent carbon atoms have been replaced by nitrogen, oxygen, or sulfur. In some embodiments, the heteroalkenyl group can be further substituted with 1, 2, 3, or 4 substituent groups as described herein for alkenyl groups. Examples of heteroalkenyl groups are an “alkenoxy” which, as used herein, refers alkenyl-O—. A heteroalkenylene is a divalent heteroalkenyl group.

The term “heteroalkynyl,” as used herein, refers to an alkynyl group, as defined herein, in which one or more of the constituent carbon atoms have been replaced by nitrogen, oxygen, or sulfur. In some embodiments, the heteroalkynyl group can be further substituted with 1, 2, 3, or 4 substituent groups as described herein for alkynyl groups. Examples of heteroalkynyl groups are an “alkynoxy” which, as used herein, refers alkynyl-O—. A heteroalkynylene is a divalent heteroalkynyl group.

The term “heteroaryl,” as used herein, refers to an aromatic mono- or polycyclic radical of 5 to 12 atoms having at least one aromatic ring containing one, two, or three ring heteroatoms selected from N, O, and S, with the remaining ring atoms being C. One or two ring carbon atoms of the heteroaryl group may be replaced with a carbonyl group. Examples of heteroaryl groups are pyridyl, pyrazoyl, benzooxazolyl, benzoimidazolyl, benzothiazolyl, imidazolyl, oxaxolyl, and thiazolyl.

The term “heteroarylalkyl,” as used herein, represents an alkyl group substituted with a heteroaryl group. Exemplary unsubstituted heteroarylalkyl groups are from 7 to 30 carbons (e.g., from 7 to 16 or from 7 to 20 carbons, such as C₂-C₉ heteroaryl C₁₋₆ alkyl, C₂-C₉ heteroaryl C₁₋₁₀ alkyl, or C₁₋₂₀ C₂-C₉ heteroaryl alkyl).

In some embodiments, the alkyl and the heteroaryl each can be further substituted with 1, 2, 3, or 4 substituent groups as defined herein for the respective groups.

The term “heterocyclyl,” as used herein, denotes a mono- or polycyclic radical having 3 to 12 atoms having at least one ring containing one, two, three, or four ring heteroatoms selected from N, O or S, wherein no ring is aromatic. Examples of heterocyclyl groups include, but are not limited to, morpholinyl, thiomorpholinyl, furyl, piperazinyl, piperidinyl, pyranyl, pyrrolidinyl, tetrahydropyranyl, tetrahydrofuranyl, and 1,3-dioxanyl.

The term “heterocyclylalkyl,” as used herein, represents an alkyl group substituted with a heterocyclyl group. Exemplary unsubstituted heterocyclylalkyl groups are from 7 to 30 carbons (e.g., from 7 to 16 or from 7 to 20 carbons, such as C₂-C₉ heterocyclyl C₁₋₆ alkyl, C₂-C₉ heterocyclyl C₁-C₁₀ alkyl, or C₂-C₉ heterocyclyl C₁-C₂₀ alkyl). In some embodiments, the akyl and the heterocyclyl each can be further substituted with 1, 2, 3, or 4 substituent groups as defined herein for the respective groups.

The term “hydroxyl,” as used herein, represents an —OH group.

The term “N-protecting group,” as used herein, represents those groups intended to protect an amino group against undesirable reactions during synthetic procedures. Commonly used N-protecting groups are disclosed in Greene, “Protective Groups in Organic Synthesis,” 3^{rd} Edition (John Wiley & Sons, New York, 1999). N-protecting groups include acyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, 4-nitrobenzoyl, and chiral auxiliaries such as protected or unprotected D, L or D, L-amino acids such as alanine, leucine, and phenylalanine; sulfonyl-containing groups such as benzenesulfonyl, and p-toluenesulfonyl; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, and phenylthiocarbonyl, arylalkyl groups such as benzyl, triphenylmethyl, and benzyloxymethyl, and silyl groups, such as trimethylsilyl. Preferred N-protecting groups are alloc, formyl, acetyl, benzoyl, pivaloyl, t-butylacetyl, alanyl, phenylsulfonyl, benzyl, t-butyloxycarbonyl (Boc), and benzyloxycarbonyl (Cbz).

The term “nitro,” as used herein, represents an —NO₂ group.

The term “thiol,” as used herein, represents an —SH group.

The alkyl, alkenyl, alkynyl, heteroalkyl, heteroalkenyl, heteroalkynyl, carbocyclyl (e.g., cycloalkyl), aryl, heteroaryl, and heterocyclyl groups may be substituted or unsubstituted. When substituted, there will generally be 1 to 4 substituents present, unless otherwise specified. Substituents include, for example: alkyl (e.g., unsubstituted and substituted, where the substituents include any group described herein, e.g., aryl, halo, hydroxy), aryl (e.g., substituted and unsubstituted phenyl), carbocyclyl (e.g., substituted and unsubstituted cycloalkyl), halogen (e.g., fluoro), hydroxyl, heteroalkyl (e.g., substituted and unsubstituted methoxy, ethoxy, or thioalkoxy), heteroaryl, heterocyclyl, amino (e.g., NH₂ or mono- or dialkyl amino), azido, cyano, nitro, or thiol. Another exemplary substituent is oxo. For example, a carbonyl group is a carbon (e.g., alkyl carbon, alkenyl carbon, alkynyl carbon, heteroalkyl carbon, heteroalkenyl carbon, heteroalkynyl carbon, carbocyclyl carbon, etc.) substituted with oxo. Alternatively, sulfur may be substituted with one or two oxo groups (e.g., —SO— or —SO₂—within a substituted heteroalkyl, heteroalkenyl, heteroalkynyl, or heterocyclyl group). Aryl, carbocyclyl (e.g., cycloalkyl), heteroaryl, and heterocyclyl groups may also be substituted with alkyl (unsubstituted and substituted such as arylalkyl (e.g., substituted and unsubstituted benzyl)).

Compounds of the invention can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates or mixtures of diastereoisomeric racemates. The optically active forms can be obtained for example by resolution of the racemates, by asymmetric synthesis or asymmetric chromatography (chromatography with a chiral adsorbents or eluant). That is, certain of the disclosed compounds may exist in various stereoisomeric forms.

Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of a compound can be prepared, for example, by separating an enantiomer from a racemate using one or more well-known techniques and methods, such as, for example, chiral chromatography and separation methods based thereon. The appropriate technique and/or method for separating an enantiomer of a compound described herein from a racemic mixture can be readily determined by those of skill in the art. “Racemate” or “racemic mixture” means a compound containing two enantiomers, wherein such mixtures exhibit no optical activity; i.e., they do not rotate the plane of polarized light. “Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds of the invention may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods. When the stereochemistry of a disclosed compound is named or depicted by structure, the named or depicted stereoisomer is at least 60%, 70%, 80%, 90%, 99% or 99.9%) by weight relative to the other stereoisomers. When a single enantiomer is named or depicted by structure, the depicted or named enantiomer is at least 60%, 70%, 80%, 90%, 99% or 99.9% by weight optically pure. When a single diastereomer is named or depicted by structure, the depicted or named diastereomer is at least 60%, 70%, 80%, 90%, 99% or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers. When the stereochemistry of a disclosed compound is named or depicted by structure, the named or depicted stereoisomer is at least 60%, 70%, 80%, 90%, 99% or 99.9% by mole fraction pure relative to the other stereoisomers. When a single enantiomer is named or depicted by structure, the depicted or named enantiomer is at least 60%, 70%, 80%, 90%, 99% or 99.9% by mole fraction pure. When a single diastereomer is named or depicted by structure, the depicted or named diastereomer is at least 60%, 70%, 80%, 90%, 99% or 99.9% by mole fraction pure. Percent purity by mole fraction is the ratio of the moles of the enantiomer or over the moles of the enantiomer plus the moles of its optical isomer. Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The invention embraces all of these forms.

### Definitions

In this application, unless otherwise clear from context, (i) the term “a” may be understood to mean “at least one”; (ii) the terms “comprising” and “including” may be understood to encompass itemized components or steps whether presented by themselves or together with one or more additional components or steps; and (iii) the term “approximately” may be understood to permit standard variation as would be understood by those of ordinary skill in the art; and (iv) where ranges are provided, endpoints are included.

As used herein, the term “about” represents a value that is in the range of ±10% of the value that follows the term “about.” Reference to “about” a value or parameter herein includes (and describes) variations that are directed to that value or parameter per se. For example, a description referring to “about X” includes the description of “X”.

As used herein, the term “administration” refers to the administration of a composition (e.g., a compound or a preparation that includes compound 1) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal, and vitreal.

An “effective amount” of a compound (e.g., compound 1) may vary according to factors such as the disease state, age, sex, and weight of the individual, and the ability of the compound to elicit the desired response. A therapeutically effective amount encompasses an amount in which any toxic or detrimental effects of the compound are outweighed by the therapeutically beneficial effects. An effective amount also encompasses an amount sufficient to confer benefit, e.g., clinical benefit.

In the practice of the methods of the present invention, an “effective amount” of any one of the compounds of the invention or a combination of any of the compounds of the invention or a pharmaceutically acceptable salt thereof, is administered via any of the usual and acceptable methods known in the art, either singly or in combination.

The term “pharmaceutical composition,” as used herein, represents a composition containing a compound described herein formulated with a pharmaceutically acceptable excipient, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other pharmaceutically acceptable formulation.

A “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

As used herein, the term “pharmaceutically acceptable salt” means any pharmaceutically acceptable salt of the compound of formula (I). For example pharmaceutically acceptable salts of any of the compounds described herein include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting a free base group with a suitable organic acid.

The compounds of the invention may have ionizable groups so as to be capable of preparation as pharmaceutically acceptable salts. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts may be prepared from pharmaceutically acceptable non-toxic acids and bases including inorganic and organic acids and bases.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

As used herein, a “Sec61-associated disorder” is a disorder caused by a polypeptide that is translocated through Sec61.

As used herein, the term “selective” refers to the preferential inhibition of a targeted process (e.g., the translocation of a given protein through a protein secretory complex such as Sec-61) rather than a non-targeted process. Compounds of the present disclosure that are selective may inhibit a targeted process at least 10-fold lower concentration than a non-targeted process (e.g., compounds that are selective may inhibit a targeted process at least 10-fold, at least 20-fold, at least 50-fold, at least 100-fold, at least 1,000 fold, at least 10,000 fold, at least 100,000-fold, at least 1,000,000-fold lower concentration than a non-targeted process). A compound that is selective may be described as having “pharmacologically appropriate selectivity” if, over the course of administration to a subject, it is sufficiently selective to inhibit a targeted process in a subject (e.g., the translocation of a disease-related protein through Sec61) while being tolerated by the subject.

As used herein, the term “subject” refers to any organism to which a composition in accordance with the invention may be administered, e.g., for experimental, diagnostic, prophylactic, and/or therapeutic purposes. Typical subjects include any animal (e.g., mammals such as mice, rats, rabbits, non-human primates, and humans). A subject may seek or be in need of treatment, require treatment, be receiving treatment, be receiving treatment in the future, or be a human or animal who is under care by a trained professional for a particular disease or condition.

As used herein, the terms “treat,” “treated,” or “treating” mean both therapeutic treatment and prophylactic or preventative measures wherein the object is to prevent or slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

As used herein, the term “translocation” refers to a process by which proteins move between cellular compartments.

Unless otherwise defined, all technical and scientific terms used herein have the same meaning as commonly understood by one of ordinary skill in the art to which this invention belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the Description below. Other features, objects, and advantages of the invention will be apparent from the Description and from the claims.

### DETAILED DESCRIPTION OF THE INVENTION

The invention features compounds useful for the selective inhibition of protein secretion, e.g., by selectively inhibiting the Sec61 protein secretion complex. Exemplary compounds described herein include compounds having a structure according to formula I:

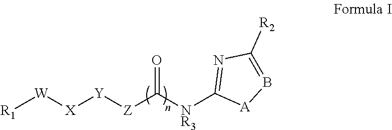

Or pharmaceutically acceptable salts thereof.

Other embodiments, as well as exemplary compounds, methods for the synthesis or production of these compounds, the use of these compounds in the treatment of a disease and/or disorder associated with Sec61, and the use of these compounds in the selective inhibition of Sec61 are described herein.

### Sec61 Inhibitors

Exemplary Sec61 inhibitors disclosed herein include a compound of Formula 1:

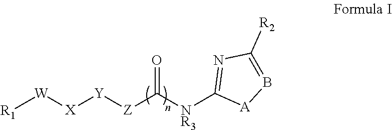

wherein
R₁ is H, optionally substituted C₁-C₆ alkyl, optionally substituted C₃-C₇ cycloalkyl, optionally substituted C₂-C₉ heterocycle, optionally substituted C₆-C₁₀ aryl, optionally substituted C₂-C₉ heteroaryl, C₂-C₉ heteroaryl C₁-C₆ alkyl, or Rₐ-R_{b}-R_{c},
   wherein Rₐ is C₆-C₁₀ arylene
   R_{b} is —O— or optionally substituted C₁-C₆ alkylene, and
R_{c} is C₆-C₁₀ aryl;
Z is absent, optionally substituted C₃-C₁₀ cycloalkylene, optionally substituted C₂-C₉ heterocyclylene, C₆-C₁₀ arylene, or optionally substituted C₂-C₉ heteroarylene;
Y is absent, O, or NR₃;
X is absent or optionally substituted C₁-C₆ alkylene;
W is absent, S, O or NR₃;
n is 0 or 1;
A is S, O, or CH;
B is C or N;
R₂ is optionally substituted C₁₋₆ alkyl, optionally substituted C₂-C₆ alkenyl, optionally substituted C₃₋₇ cycloalkyl, optionally substituted C₂₋₉ heterocycle, optionally substituted C₆-C₁₀ aryl, optionally substituted C₂₋₉ heteroaryl; optionally substituted C₂-C₉ heteroaryl C₁-C₆ alkyl, optionally substituted C₃-C₇ cycloalkyl C₁-C₆ aryl, optionally substituted C₁-C₆ heteroalkyl, or cyano; and
each R₃ is, independently, H or C₁-C₆ alkyl.

Exemplary Sec61 inhibitors described herein include a compound of Formula II:

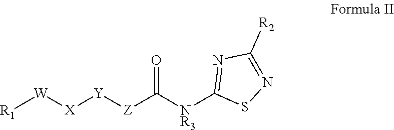

Exemplary Sec61 inhibitors described herein include a compound of Formula III:

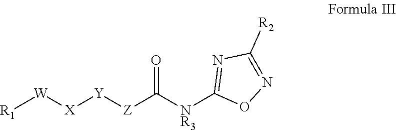

Exemplary Sec61 inhibitors described herein include a compound of Formula IV:

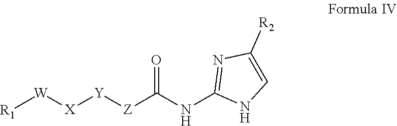

Exemplary Sec61 inhibitors described herein include a compound of Formula V:

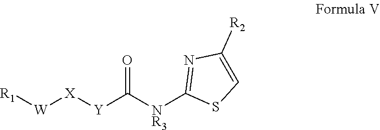

wherein Y is O or NR₃.

Exemplary Sec61 inhibitors described herein include a compound of Formula VI:

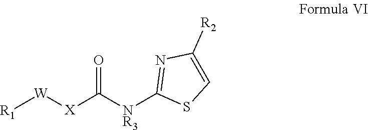

wherein W is NR₃.

Exemplary Sec61 inhibitors described herein include a compound of Formula VII:

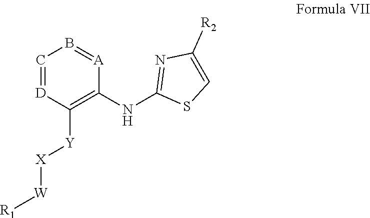

wherein A, B, C and D are, each, independently, N, CH, or CR₄; and
each R₄ is independently optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol.

Exemplary Sec61 inhibitors described herein include a compound of Formula VIII:

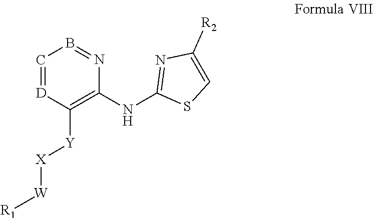

wherein B, C, and D are each, independently, CH or CR₄

Exemplary Sec61 inhibitors described herein include a compound of Formula IX:

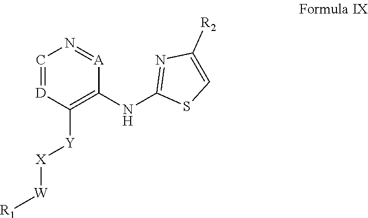

wherein A, C, and D are each, independently, CH or CR₄.

Exemplary Sec61 inhibitors described herein include a compound of Formula X:

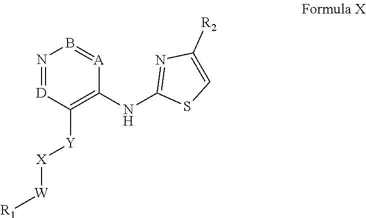

wherein A, B, and D are each, independently, CH or CR₄

Exemplary Sec61 inhibitors described herein include a compound of Formula XI:

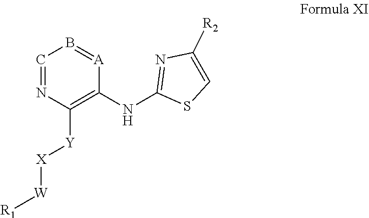

wherein A, B, and C are each, independently, CH or CR₄.

Exemplary Sec61 inhibitors described herein include a compound of Formula XII:

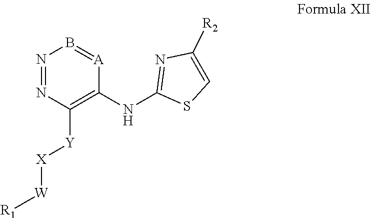

wherein A and B are each, independently, CH or CR₄.

Exemplary Sec61 inhibitors described herein include a compound of Formula XIII:

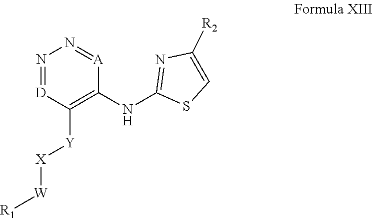

wherein A and D are each, independently, CH or CR₄.

Exemplary Sec61 inhibitors described herein include a compound of Formula XIV:

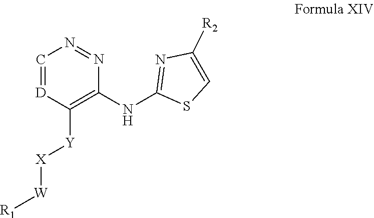

wherein C and D are each, independently, CH or CR₄.

Exemplary Sec61 inhibitors described herein include a compound of Formula XV:

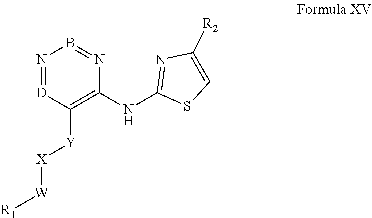

wherein B and D are each, independently, CH or CR₄.

Exemplary Sec61 inhibitors described herein include a compound of Formula XVI:

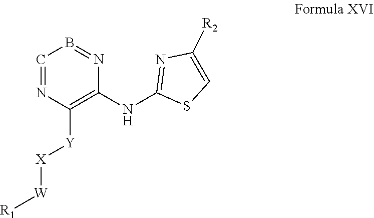

wherein B and C are each, independently, CH or CR₄.

Exemplary Sec61 inhibitors described herein include a compound of Formula XVII:

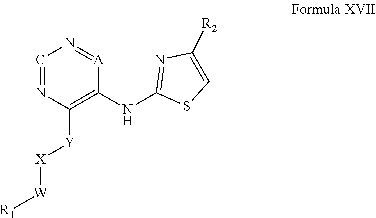

wherein A and C are each, independently, CH or CR₄.

Exemplary Sec61 inhibitors described herein include a compound of Formula XVIII:

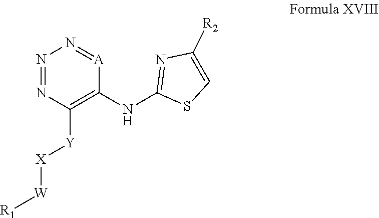

wherein A is CH or CR₄.

Exemplary Sec61 inhibitors described herein include a compound of Formula XIX:

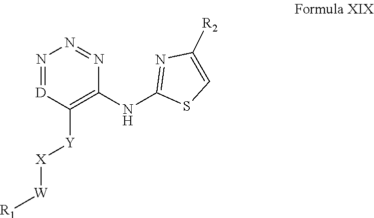

wherein D Is CH or CR₄.

Exemplary Sec61 inhibitors described herein include a compound of Formula XX:

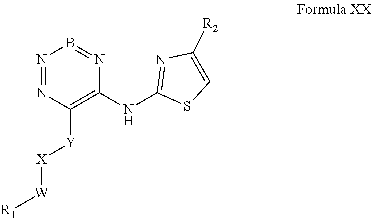

wherein B is CH or CR₄.

Exemplary Sec61 inhibitors described herein include a compound of Formula XXI:

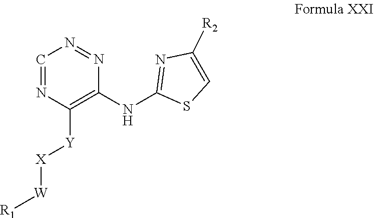

wherein C is CH or CR₄.

Exemplary Sec61 inhibitors described herein include a compound of Formula XXII:

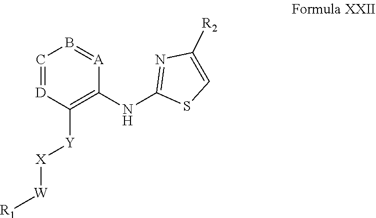

wherein A, B, C, and D are each, independently, CH or CR₄

Exemplary Sec61 inhibitors described herein include a compound of Formula XXIII:

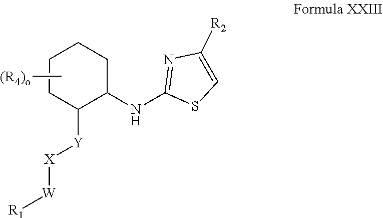

wherein o is 0, 1, 2, 3, or 4; and
each R₄ is, independently, optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol.

Exemplary Sec61 inhibitors described herein include a compound of Formula XXIV:

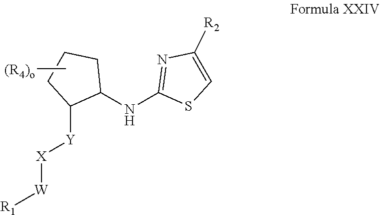

wherein o is 0, 1, 2, or 3; and
each R₄ is, independently, optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol.

Exemplary Sec61 inhibitors described herein include a compound of Formula XXV:

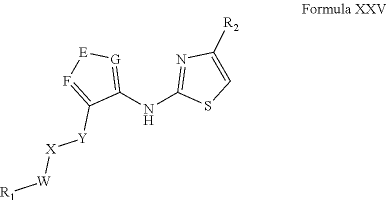

wherein E is NH, O, or S;
F and G are each, independently, N, CH, or CR₄; and
each R₄ is independently optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol.

Exemplary Sec61 inhibitors described herein include a compound of Formula XXVI:

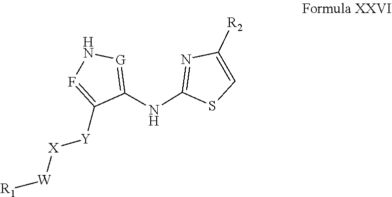

wherein F and G are each, independently, CH or CR₄.

Exemplary Sec61 inhibitors described herein include a compound of Formula XXVII:

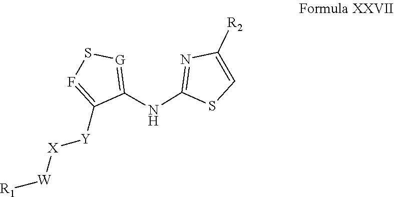

wherein F and G are each, independently, CH or CR₄

Exemplary Sec61 inhibitors described herein include a compound of Formula XXVIII:

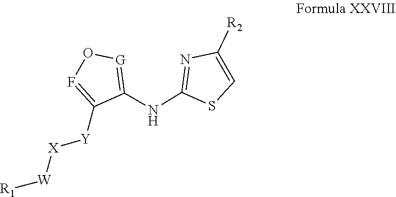

wherein F and G are each, independently, CH or CR₄.

Exemplary Sec61 inhibitors described herein include a compound of Formula XXIX:

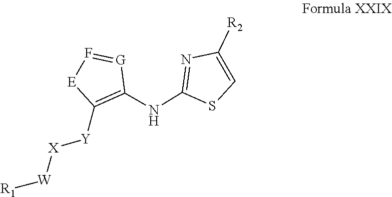

wherein E is NH, O, or S;
F and G are each, independently, N, CH, or CR₄; and
each R₄ is independently optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol.

Exemplary Sec61 inhibitors described herein include a compound of Formula XXX:

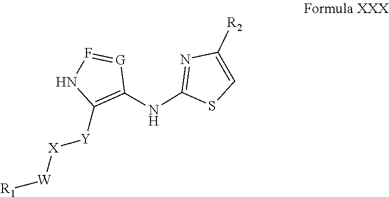

wherein F and G are each, independently, CH or CR₄.

Exemplary Sec61 inhibitors described herein include a compound of Formula XXXI:

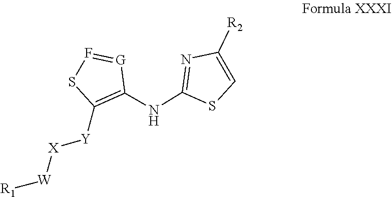

wherein F and G are each, independently, CH or CR₄.

Exemplary Sec61 inhibitors described herein include compounds of Formula XXXII:

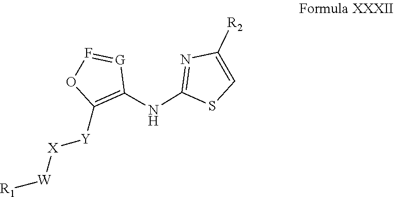

wherein F and G are each, independently, CH or CR₄.

Exemplary Sec61 inhibitors described herein include compounds of Formula XXXIII:

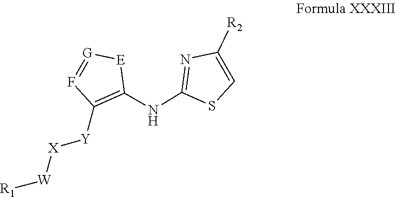

wherein
E is NH, O, or S;
F and G are each, independently, N, CH, or CR₄; and
each R₄ is independently optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol.

Exemplary Sec61 inhibitors described herein include compounds of Formula XXXIV:

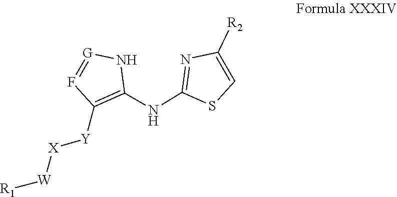

wherein F and G are each, independently, CH or CR₄.

Exemplary Sec61 inhibitors described herein include compounds of Formula XXXV:

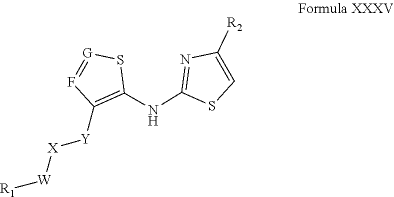

wherein F and G are each, independently, CH or CR₄.

Exemplary Sec61 inhibitors described herein include compounds of Formula XXXVI:

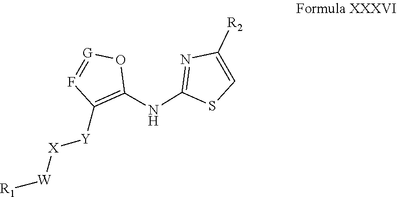

wherein F and G are each, independently, CH or CR₄.

Exemplary Sec61 inhibitors described herein include compounds of Formula XXXVII:

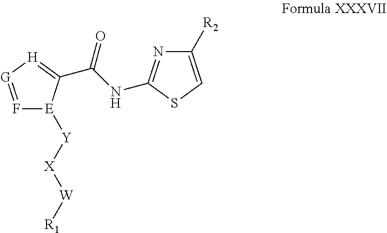

wherein E is N or CH;
F, G, and H are each, independently, N, CH, or CR₄; and
each R₄ is independently optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol.

Exemplary Sec61 inhibitors described herein include compounds of Formula XXXVIII:

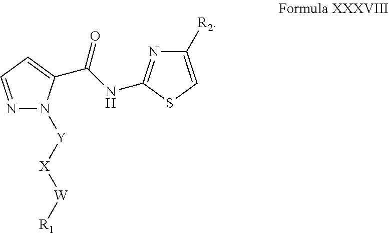

Exemplary Sec61 inhibitors described herein include compounds of Formula XXXIX:

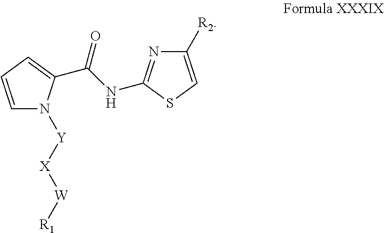

Exemplary Sec61 inhibitors described herein include compounds of Formula XXXX:

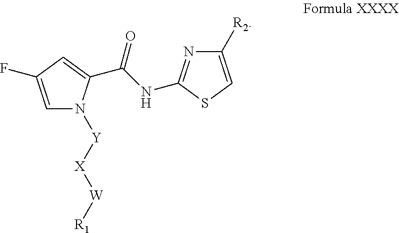

Exemplary Sec61 inhibitors described herein include compounds of Formula XXXXI:

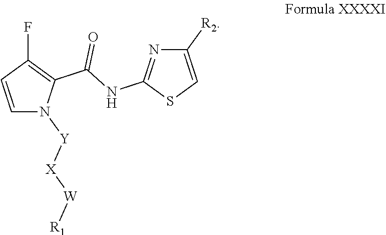

Exemplary Sec61 inhibitors described herein include compounds of Formula XXXXII:

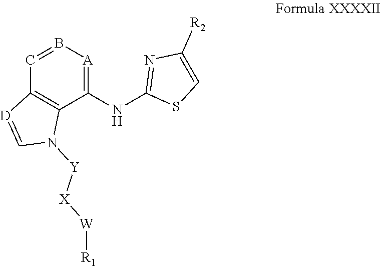

wherein A, B, C, and D are each, independently, CH, CR₄, or N; and
each R₄ is independently optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol.

Exemplary Sec61 inhibitors described herein include compounds of Formula XXXXIII:

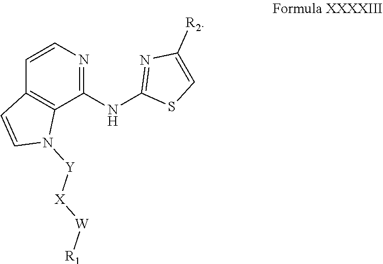

Sec61 inhibitors described here in include any one of the compounds in Table 1.

TABLE 1
Compounds of the Invention

| # | Structure |
|---|---|
| 1 | 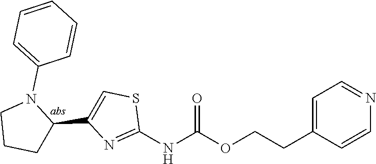 [SMILES: O=C(Nc1nc([C@H]2CCCN2c2ccccc2)cs1)OCCc1ccncc1] |
| 2 | 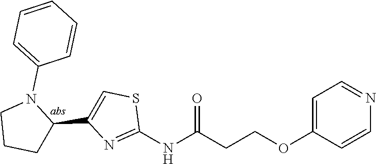 [SMILES: O=C(CCOc1ccncc1)Nc1nc([C@H]2CCCN2c2ccccc2)cs1] |
| 3 | 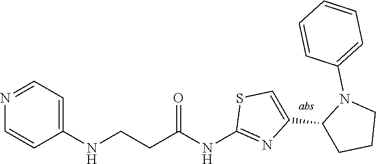 [SMILES: O=C(CCNc1ccncc1)Nc1nc([C@H]2CCCN2c2ccccc2)cs1] |
| 4 | 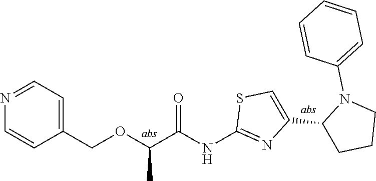 [SMILES: C[C@@H](OCc1ccncc1)C(=O)Nc1nc([C@H]2CCCN2c2ccccc2)cs1] |
| 5 | 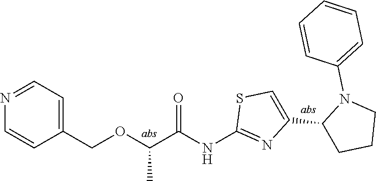 [SMILES: C[C@H](OCc1ccncc1)C(=O)Nc1nc([C@H]2CCCN2c2ccccc2)cs1] |
| 6 | 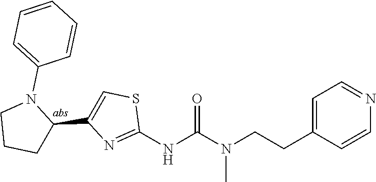 [SMILES: CN(CCc1ccncc1)C(=O)Nc1nc([C@H]2CCCN2c2ccccc2)cs1] |
| 7 | 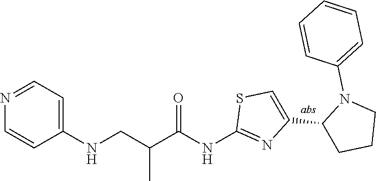 [SMILES: CC(CNc1ccncc1)C(=O)Nc1nc([C@H]2CCCN2c2ccccc2)cs1] |
| 8 | 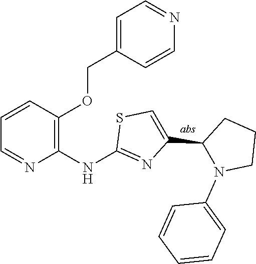 [SMILES: c1ccc(N2CCC[C@@H]2c2csc(Nc3ncccc3OCc3ccncc3)n2)cc1] |
| 9 | 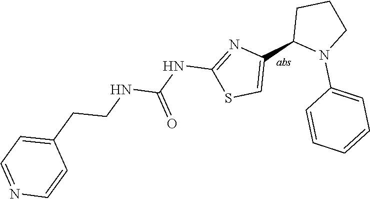 [SMILES: O=C(NCCc1ccncc1)Nc1nc([C@H]2CCCN2c2ccccc2)cs1] |
| 10 | 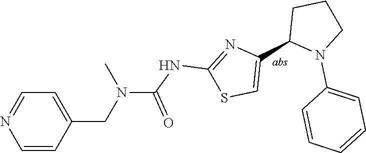 [SMILES: CN(Cc1ccncc1)C(=O)Nc1nc([C@H]2CCCN2c2ccccc2)cs1] |
| 11 | 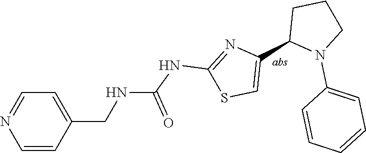 [SMILES: O=C(NCc1ccncc1)Nc1nc([C@H]2CCCN2c2ccccc2)cs1] |
| 12 | 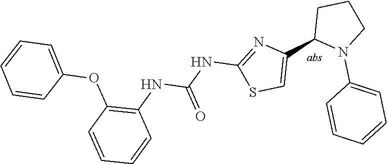 [SMILES: O=C(Nc1nc([C@H]2CCCN2c2ccccc2)cs1)Nc1ccccc1Oc1ccccc1] |
| 13 | 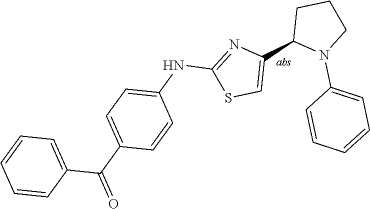 [SMILES: O=C(c1ccccc1)c1ccc(Nc2nc([C@H]3CCCN3c3ccccc3)cs2)cc1] |
| 14 | 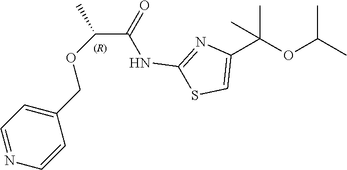 |
| 15 | 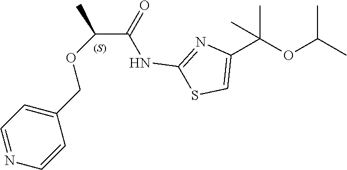 |
| 16 | 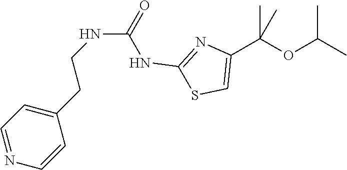 [SMILES: CC(C)OC(C)(C)c1csc(NC(=O)NCCc2ccncc2)n1] |
| 17 | 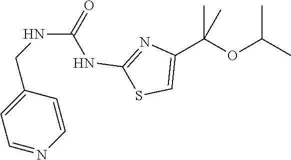 [SMILES: CC(C)OC(C)(C)c1csc(NC(=O)NCc2ccncc2)n1] |
| 18 | 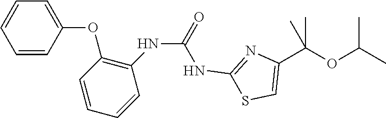 [SMILES: CC(C)OC(C)(C)c1csc(NC(=O)Nc2ccccc2Oc2ccccc2)n1] |
| 19 | 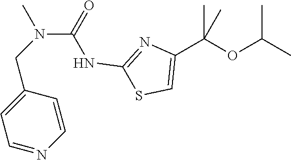 [SMILES: CC(C)OC(C)(C)c1csc(NC(=O)N(C)Cc2ccncc2)n1] |
| 20 | 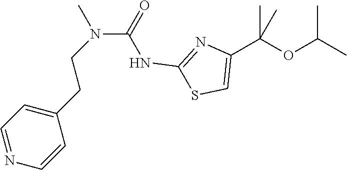 [SMILES: CC(C)OC(C)(C)c1csc(NC(=O)N(C)CCc2ccncc2)n1] |
| 21 | 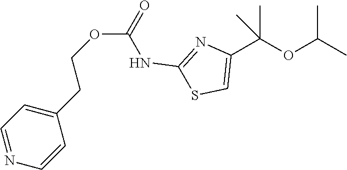 [SMILES: CC(C)OC(C)(C)c1csc(NC(=O)OCCc2ccncc2)n1] |
| 22 | 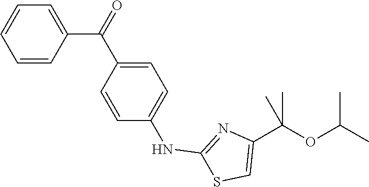 [SMILES: CC(C)OC(C)(C)c1csc(Nc2ccc(C(=O)c3ccccc3)cc2)n1] |
| 23 | 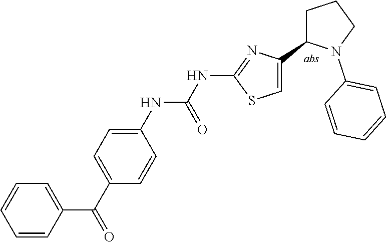 [SMILES: O=C(Nc1ccc(C(=O)c2ccccc2)cc1)Nc1nc([C@H]2CCCN2c2ccccc2)cs1] |
| 24 | 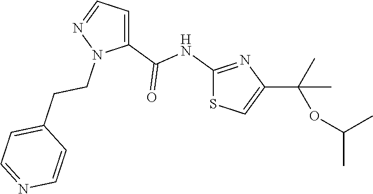 [SMILES: CC(C)OC(C)(C)c1csc(NC(=O)c2ccnn2CCc2ccncc2)n1] |
| 25 | 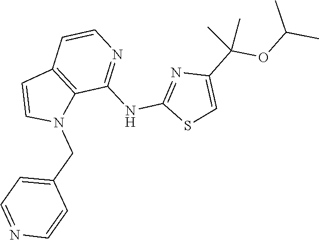 [SMILES: CC(C)OC(C)(C)c1csc(Nc2nccc3ccn(Cc4ccncc4)c23)n1] |
| 26 | 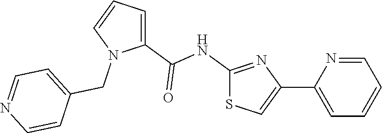 [SMILES: O=C(Nc1nc(-c2ccccn2)cs1)c1cccn1Cc1ccncc1] |
| 27 | 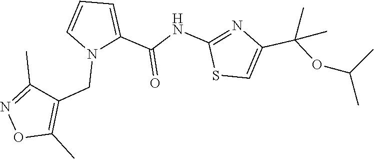 [SMILES: Cc1noc(C)c1Cn1cccc1C(=O)Nc1nc(C(C)(C)OC(C)C)cs1] |
| 28 | 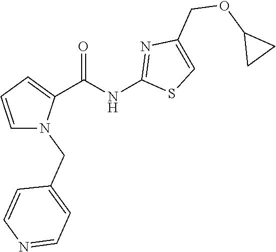 [SMILES: O=C(Nc1nc(COC2CC2)cs1)c1cccn1Cc1ccncc1] |
| 29 | 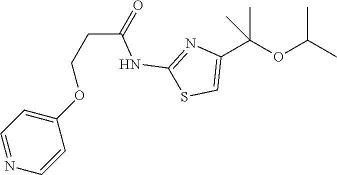 [SMILES: CC(C)OC(C)(C)c1csc(NC(=O)CCOc2ccncc2)n1] |
| 30 | 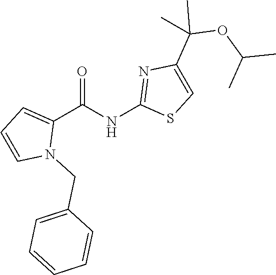 [SMILES: CC(C)OC(C)(C)c1csc(NC(=O)c2cccn2Cc2ccccc2)n1] |
| 31 | 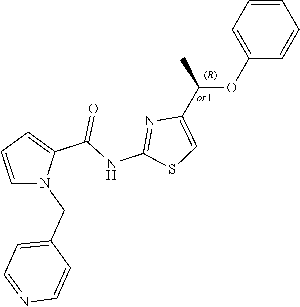 |
| 32 | 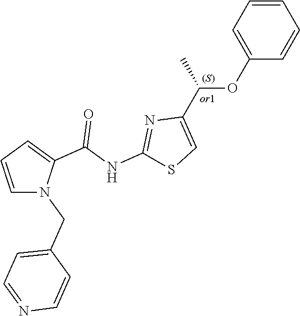 |
| 33 | 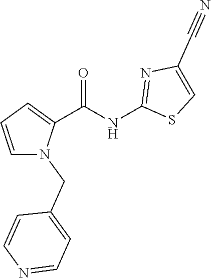 [SMILES: N#Cc1csc(NC(=O)c2cccn2Cc2ccncc2)n1] |
| 35 | 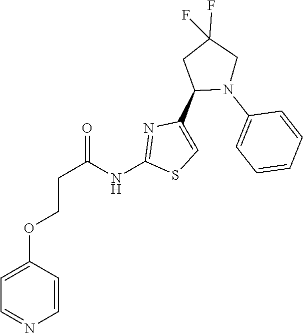 [SMILES: O=C(CCOc1ccncc1)Nc1nc([C@H]2CC(F)(F)CN2c2ccccc2)cs1] |
| 36 | 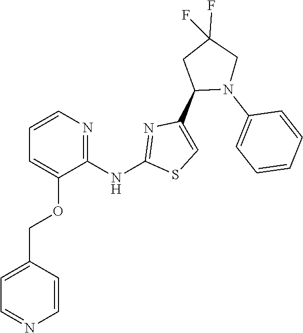 [SMILES: FC1(F)C[C@H](c2csc(Nc3ncccc3OCc3ccncc3)n2)N(c2ccccc2)C1] |
| 37 | 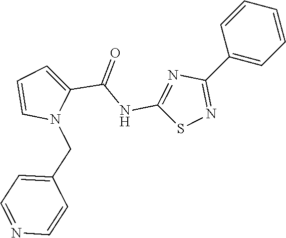 [SMILES: O=C(Nc1nc(-c2ccccc2)ns1)c1cccn1Cc1ccncc1] |
| 38 | 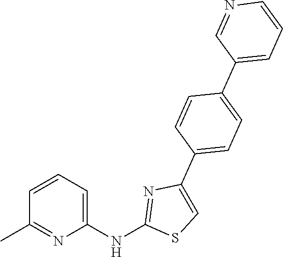 [SMILES: Cc1cccc(Nc2nc(-c3ccc(-c4cccnc4)cc3)cs2)n1] |
| 39 | 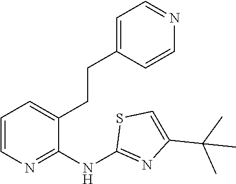 [SMILES: CC(C)(C)c1csc(Nc2ncccc2CCc2ccncc2)n1] |
| 43 | 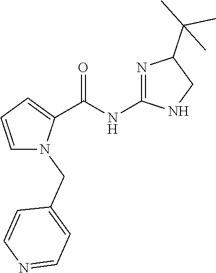 [SMILES: CC(C)(C)C1CNC(NC(=O)c2cccn2Cc2ccncc2)=N1] |
| 44 | 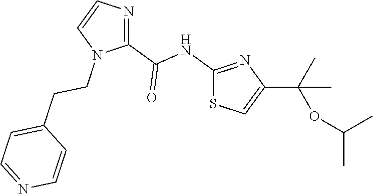 [SMILES: CC(C)OC(C)(C)c1csc(NC(=O)c2nccn2CCc2ccncc2)n1] |
| 45 | 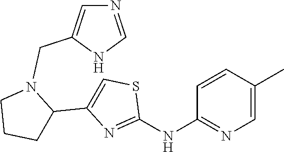 [SMILES: Cc1ccc(Nc2nc(C3CCCN3Cc3cnc[nH]3)cs2)nc1] |
| 46 | 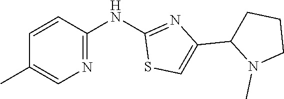 [SMILES: Cc1ccc(Nc2nc(C3CCCN3C)cs2)nc1] |
| 47 | 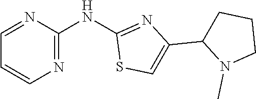 [SMILES: CN1CCCC1c1csc(Nc2ncccn2)n1] |
| 48 | 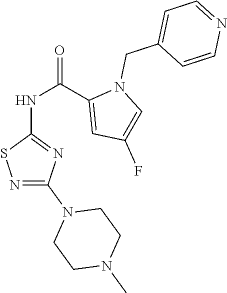 [SMILES: CN1CCN(c2nsc(NC(=O)c3cc(F)cn3Cc3ccncc3)n2)CC1] |
| 49 | 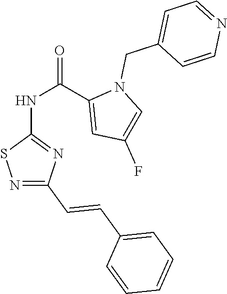 [SMILES: O=C(Nc1nc(/C=C/c2ccccc2)ns1)c1cc(F)cn1Cc1ccncc1] |
| 50 | 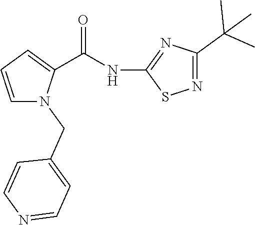 [SMILES: CC(C)(C)c1nsc(NC(=O)c2cccn2Cc2ccncc2)n1] |

### Pharmaceutical Compositions

The compounds of the invention are preferably formulated into pharmaceutical compositions for administration to human subjects in a biologically compatible form suitable for administration in vivo.

Accordingly, in another aspect, the present invention provides a pharmaceutical composition comprising a compound of the invention in admixture with a suitable diluent, carrier, or excipient.

The compounds of the invention may be used in the form of the free base, in the form of salts, solvates, and as prodrugs. All forms are within the scope of the invention. In accordance with the methods of the invention, the described compounds or salts, solvates, or prodrugs thereof may be administered to a patient in a variety of forms depending on the selected route of administration, as will be understood by those skilled in the art. The compounds of the invention may be administered, for example, by oral, parenteral, buccal, sublingual, nasal, rectal, patch, pump, or transdermal administration and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

A compound of the invention may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, a compound of the invention may be incorporated with an excipient and used in the form of ingestible tablets, buccal tablets, troches, capsules, elixirs, suspensions, syrups, and wafers.

A compound of the invention may also be administered parenterally. Solutions of a compound of the invention can be prepared in water suitably mixed with a surfactant, such as hydroxypropylcellulose. Dispersions can also be prepared in glycerol, liquid polyethylene glycols, DMSO and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington's Pharmaceutical Sciences (2003, 20^{th} ed.) and in The United States Pharmacopeia: The National Formulary (USP 24 NF19), published in 1999.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that may be easily administered via syringe.

Compositions for nasal administration may conveniently be formulated as aerosols, drops, gels, and powders. Aerosol formulations typically include a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, such as a single dose nasal inhaler or an aerosol dispenser fitted with a metering valve which is intended for disposal after use. Where the dosage form comprises an aerosol dispenser, it will contain a propellant, which can be a compressed gas, such as compressed air or an organic propellant, such as fluorochlorohydrocarbon. The aerosol dosage forms can also take the form of a pump-atomizer.

Compositions suitable for buccal or sublingual administration include tablets, lozenges, and pastilles, where the active ingredient is formulated with a carrier, such as sugar, acacia, tragacanth, gelatin, and glycerine.

Compositions for rectal administration are conveniently in the form of suppositories containing a conventional suppository base, such as cocoa butter.

The compounds of the invention may be administered to an animal, e.g., a human, alone or in combination with pharmaceutically acceptable carriers, as noted herein, the proportion of which is determined by the solubility and chemical nature of the compound, chosen route of administration, and standard pharmaceutical practice.

### Dosages

The dosage of the compounds of the invention, and/or compositions comprising a compound of the invention, can vary depending on many factors, such as the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The compounds of the invention may be administered initially in a suitable dosage that may be adjusted as required, depending on the clinical response.

### Protein Secretion

Myriad proteins are secreted from cells. In humans, secreted proteins are trafficked from the cytosol to outside the cell by the protein secretory pathway. The proteins secretory pathway comprises the endoplasmic reticulum, Golgi bodies, secretory or transport vesicles, and the cell membrane. In order to be secreted from cells, proteins must pass through the biological membranes that divide cells into compartments. The passage of proteins from the cytosol into the membrane of the endoplasmic reticulum is facilitated by the translocon, a complex of proteins comprising at least Sec61. Polypeptides which are secreted from cells may comprise a signal peptide. The signal peptide is a sequence of amino acids located at the N-terminus of the polypeptide to be secreted. The signal peptide facilitates targeting of the polypeptide to the translocon, e.g., targeting to Sec61. In some instances, after translocation through the translocon, e.g., through Sec61, the signal peptide is cleaved from the polypeptide, generating a free signal peptide and a mature polypeptide.

### Inhibition of Sec61

Sec61 is a membrane protein complex that, in humans, translocates nascent proteins from the cytosol into the endoplasmic reticulum. Sec61 is a hetero-trimeric complex comprising three subunits: SecY, SecE, and SecG. Newly synthesized polypeptides pass from the ribosome, which associates with Sec61, through Sec61 and into the membrane of the endoplasmic reticulum. Sec61 comprises a channel through which polypeptides pass, and a luminal plug, which, when closed, blocks passage through the channel. The luminal plug is displaced, opening the channel, when polypeptides interact with other portions of Sec61.

The present inventors have discovered small molecules that selectively inhibit the translocation of specific polypeptides through Sec61. The following description of the Sec61 inhibitors described herein is provided without wishing to be bound by theory. In some embodiments, the Sec61 inhibitors of the present disclosure bind to the luminal plug region of Sec61. In some embodiments, a conserved portion of the Sec61 inhibitors binds to Sec61. In some embodiments, a variable portion of the Sec61 inhibitors makes contacts with the signal peptide of a nascent polypeptide that is in the process of translocating through Sec61. In some embodiments, the contacts between the variable portion of the Sec61 inhibitor and the signal peptide of the nascent polypeptide prevent the translocation of the polypeptide. In some embodiments, selectivity can be achieved for different signal peptides by chemical modification of the variable portion of the Sec61 inhibitor.

In some embodiments, the signal peptide is orthogonal and independent of the mature protein sequence. In some embodiments, targeting the signal portion of the polypeptide allows for the inhibition of targets that lack druggable handles.

In some embodiments, components of the body, e.g., the immune system, recognize cells by the proteins that are expressed on the cell surface. In some embodiments, cell identity is determined by the proteins expressed on the cell surface. In some embodiments, selectively targeting the translocation through Sec61 of proteins that are expressed on the cell surface allows for selective editing of the proteins expressed on the cell surface. In some embodiments, selectively targeting the translocation through Sec61 of proteins that are expressed on the cell surface allows for the non-destructive changes to cell identity. In some embodiments, selectively targeting the translocation through Sec61 of proteins that are expressed on the cell surface allows for the removal of surface proteins without harming the cell.

In some embodiments, selective targeting of the translocation through Sec61 of proteins allows for the targeting of tissue types that are inaccessible to other modalities, e.g., the brain (mABS) and non-liver (siRNA).

In some embodiments, selectively targeting the translocation through Sec61 of newly made proteins and cells that actively synthesize proteins allows for the targeting of dynamically-regulated targets and synthesis-sensitive cells.

In some embodiments, selectively targeting the translocation of irreversibly pathogenic proteins through Sec61 allows for targeting the aggregation of irreversibly pathogenic proteins and cascades of irreversibly pathogenic proteins.

In some embodiments, selectively targeting the translocation of viral proteins thorough Sec61 blocks viral protein secretion and inhibits viral replication.

### Methods of Treatment

The compounds described herein may be used to treat diseases and/or disorders associated with secreted proteins that are translocated through Sec61. In some embodiments, the compounds described herein selectively inhibit the translocation of a disease-associated protein through Sec61. Sec6l-associated diseases and/or disorders that may be treated by a compound described herein include, amyloidosis, light chain amyloidosis, autoantibody diseases, chronic kidney disease, fibrosis, neurodegeneration, autoimmune disease, genetically-defined kidney disease, viral disease, influenza, dengue virus, zika virus, hepatitis B virus, hepatitis C virus, SARS-CoV-2, and human immunodeficiency virus, malaria, cancer, glioma, myeloma, multiple types of cancer with solid tumors, autoimmune diseases, rheumatoid arthritis, ankylosing spondylitis, celiac disease, multiple sclerosis, atopic dermatitis, Crohn's disease, psoriasis, allergic asthma, autoimmune antibody diseases, myasthenia gravis, neuromyelitis optica, warm antibody hemolytic anemia, prion disease, immune thrombocytopenic purpura, chronic inflammatory demyelinating polyradiculoneuropathy, fibrotic diseases, idiopathic pulmonary fibrosis, endometriosis, nonalcoholic steatohepatitis, neurodegenerative diseases, Alzheimer's disease, Parkinson's disease, Huntington's disease, amyotrophic lateral sclerosis, and hypercholesterolemia, Creutzfeldt-Jakob disease, Gerstmann-Strsussler-Scheinker syndrome, high cholesterol, metabolic syndrome, and fatal familial insomnia.

### Light Chain Amyloidosis

Amyloidosis is a group of diseases characterized by the harmful and undesired buildup of misfolded amyloid protein in various tissues. Nonlimiting examples of tissues that may suffer from buildup of misfolded amyloid protein include the kidneys, the heart, the brain, the liver, the thyroid glands, the adrenal glands, the musculoskeletal system, the eyes, and the oral cavity. Light chain amyloidosis is a form of amyloidosis characterized by the buildup of amyloid protein formed from the light chains (AL) of antibodies produced in bone marrow by plasma cells. The secretion of light chain in plasma cells is facilitated by Sec61. In light chain amyloidosis, amyloid protein is deposited in tissues throughout the body including but not limited to the kidneys, the heart, the digestive system, the liver, and the nervous system. Without wishing to be bound by theory, inhibition of light chain translocation by Sec61 may reduce the levels of amyloid derived from light chains present in tissues including but not limited to the kidneys, the heart, the digestive system, the liver, and the nervous system.

### Prion Disease

Prion disease is a fatal neurodegenerative disease caused by the elevated levels of misfolded prion protein (PrP). Individuals suffering from prion disease may experience impaired brain function causing changes in memory, personality and behavior, dementia, and ataxia. PrP is translocated from the cytosol to the endoplasmic reticulum by Sec61. Without wishing to bound by theory, inhibition of PrP translocation via Sec61 may reduce circulating PrP levels, e.g., in the brain.

### Autoimmune Antibody Disease

Autoimmune Antibody Disease is characterized by the failure of the immune system to differentiate between host antigens and foreign antigens. In some instances, Autoimmune Antibody Disease comprises immunoglobulin G (IgG) having pathogenic effects on the body. The neonatal crystallizable fragment receptor (FcRn) is a protective IgG receptor and is positively correlated with the level of circulating IgG. FcRn is translocated into the endoplasmic reticulum via Sec61. Without wishing to be bound by theory, inhibition of FcRn translocation may reduce IgG levels.

### Genetically Defined Kidney Disease

Genetically defined kidney disease comprises a group of kidney disease that have a genetic origin. Nonlimiting examples of genetically-defined kidney diseases include End-stage kidney disease (ESKD) and

Focal segmental glomerulosclerosis (FSGS). APOL1 is a protein associated with genetically defined kidney diseases. APOL1 is translocated into the endoplasmic reticulum via Sec61. Without wishing to be bound by theory, inhibition of APOL1 translocation by Sec61 may reduce the levels of APOL1 in the kidney and thus preserve podocytes and slow the progression of kidney disease.

### Malaria

Malaria is a disease caused by the parasite Plasmodium falciparum. P. falciparum utilizes Sec61 to export proteins that are important for its survival and replication into host erythrocytes. Without wishing to be bound by theory, inhibition of translocation of P. falciparum proteins via Sec61 may inhibit the replication of P. falciparum.

### Viral Diseases

All viruses co-opt the cellular machinery of their host cell in order to replicate. Nonlimiting examples of viruses whose ability to replicate inside host cells may be Sec61-dependent include influenza, dengue virus, zika virus, hepatitis B virus, hepatitis C virus, SARS-CoV-2, and human immunodeficiency virus (HIV). Without wishing to be bound by theory, Sec61 may contribute to the transportation of viral proteins into biological membranes, e.g., into the endoplasmic reticulum. Without wishing to be bound by theory, inhibition of the translocation of viral proteins through Sec61 may prevent the replication of viruses in host cells.

### Cancer

Cancer is a group of diseases characterized by the harmful, abnormal, uncontrolled, and undesirable growth of cells. Cancer occurs in multiple tissues and organs, including the lungs, the breasts, the bladder, the colon, the rectum, the uterus, the testes, the kidneys, the blood, the lymphatic system, the liver, the bile ducts, the skin, the pancreas, the prostate, the thyroid gland, the brain, the spinal cord, and the stomach. Cancer cells differ from healthy cells in their protein expression profiles. For example, cancer cells may express proteins which are involved in tumor metastasis, such as CD74, at higher levels than healthy cells.

Without wishing to be bound by theory, the proteins translocated through Sec61 in cancer cells may differ from the proteins translocated through Sec61 in healthy cells. Additionally, proteins that are translocated through Sec61 in both cancer cells and healthy cells may be translocated through Sec61 at a higher rate in cancer cells than in healthy cells. Furthermore, cancer cells may rely on proteins translocated through Sec61 for survival, such as increased proliferation or evasion from immune recognition. Inhibiting the translocation of proteins that are translocated through Sec61 in cancer cells but not in healthy cells, and/or inhibiting the translocation of proteins that are translocated through Sec61 at higher levels in cancer cells than in healthy cells, and/or inhibiting the translocation of proteins that cancer cells rely on for increased proliferation or evasion from immune recognition, may inhibit the proliferation of cancer cells.

### Equivalents and Scope

Those skilled in the art will recognize or be able to ascertain using no more than routine experimentation, many equivalents to the specific embodiments in accordance with the invention described herein. The scope of the present invention is not intended to be limited to the above Description.

The following Examples are illustrative only and not intended to limit the invention in any way.

### EXAMPLES

### Example 1. Synthesis of Compounds

Compounds of the invention can be synthesized according to one or more of the exemplary syntheses shown below.

### Materials and Methods

NMR Equipment: NMR spectra were recorded at 400 MHz using a QOne AS400 400 MHz spectrometer.

### LC-MS (Aglient-P2):

LC: Agilent Technologies 1290 series, Binary Pump, Diode Array Detector. Agilent EclipsePlus RRHD C18, 1.8 μm, 3.0×50 mm. Mobile phase: A: 0.05% Formate in water (v/v), B: 0.05% Formate in MeCN(v/v). Flow Rate: 0.8 mL/min at 25° C. Detector: 214 nm, 254 nm.

### Timetable:

| T (min) | A (%) | B (%) |
|---|---|---|
| 0.00 | 90 | 10 |
| 0.50 | 90 | 10 |
| 4.00 | 10 | 90 |
| 4.50 | 0 | 100 |
| 4.51 | 90 | 10 |
| 5.00 | 90 | 10 |

### LC-MS (SHIMADZU-2020-P2):

LC: Shimadzu LC-20 AD series, Binary Pump, Diode Array Detector. Waters Sunfire, 3.5 μm, 4.6×50 mm column. Mobile phase: A: 0.05% Formate in water (v/v), B: 0.05% Formate in MeCN(v/v). Flow Rate: 1 mL/min at 25° C. Detector: 214 nm, 254 nm. Gradient stop time, 5 min. Timetable:

| T (min) | A (%) | B (%) |
|---|---|---|
| 0.00 | 85 | 15 |
| 0.50 | 85 | 15 |
| 4.00 | 0 | 100 |
| 4.50 | 0 | 100 |
| 4.51 | 85 | 15 |
| 5.00 | 85 | 15 |

1. MS: 2020, Quadrupole LC/MS, Ion Source: API-ESI, TIC: 100-900 m/z, Drying gas flow: 15 L/min, Nebulizer pressure: 1.5 L/min, Drying gas temperature: 250° C., Vcap: 4500V.
2. Sample preparation: samples were dissolved in methanol at 1-10 μg/mL, then filtered through a 0.22 μm filter membrane. Injection volume: 1-10 μL.

### HPLC (Agilent-1200-A2):

LC: Agilent Technologies 1200 series, Binary Pump, Diode Array Detector. Column Temperature: 35° C.; Acquisition wavelength: 214 nm, 254 nm; Mobile Phase A: 0.1% TFA in water (v/v); Mobile Phase B: CAN; Run time: 18.01 min; Post time: 2 min; Flow rate: 1.0 ml/min HPLC-01-A2: Gradient stop time, 18 min.

### Timetable:

| Time(min) | A | B |
|---|---|---|
| 0 | 90 | 10 |
| 1 | 90 | 10 |
| 17 | 0 | 100 |
| 18 | 0 | 100 |

### Synthesis of Intermediate 1

### Step 1: Synthesis of methyl (2R)-4,4-difluoro-1-[phenyl]pyrrolidine-2-carboxylate

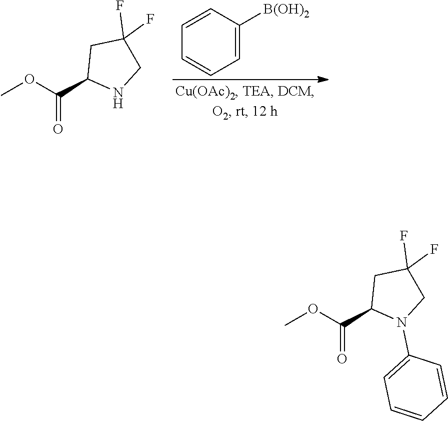

A 100 mL round bottom flask with stir bar was charged with methyl (2R)-4,4-difluoropyrrolidine-2-carboxylate (2.00 g, 4.4 mmol, 1.00 equiv), phenyl boronic acid (1.59 g, 13.0 mmol, 3 equiv), Cu(OAc)₂ (2.37 g, 13.0 mmol, 3 equiv), TEA (2.20 g, 21.8 mmol, 5 equiv) and DCM (30 mL, 0.15 M) under nitrogen atmosphere. The reaction flask was then vacuumed and flushed with oxygen, and the sequence was repeated twice. The vial was capped and placed in a 25° C. bath. The reaction mixture was stirred at 25° C. for 48 h under oxygen atmosphere using an oxygen balloon. The reaction mixture was poured into DCM (150 mL) and quenched by the addition of NH₃·H₂O (20 mL), washed with H₂O (1×50 mL) and brine (3×50 mL). The organic layer was dried over Na₂SO₄, filtered and concentrated in vacuo. The resulting crude material was purified via silica gel chromatography to yield the desired product. LCMS (+ESI): calc. [M+H]+=242; found 242.

### Step 2: synthesis of 2-(2-chloroacetyl)-4,4-difluoro-1-[phenyl]pyrrolidine

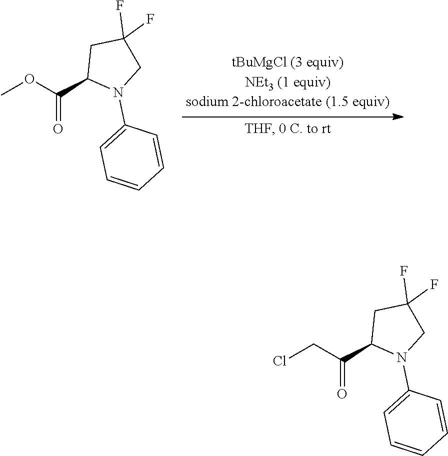 [SMILES: COC(=O)[C@H]1CC(F)(F)CN1c1ccccc1 ; O=C(CCl)[C@H]1CC(F)(F)CN1c1ccccc1]

A 20 mL vial with stir bar was charged with methyl (2R)-4,4-difluoro-1-[phenyl]pyrrolidine-2-carboxylate (182 g, 0.887 mmol, 1.0 equiv), sodium 2-chloroacetate (155 mg, 1.33 mmol, 1.5 equiv), triethylamine (0.124 mL, 0.887 mmol, 1.0 equiv) and THE (1.0 mL, 0.2 M). The reaction mixture was cooled to 0° C., and tert-butylmagnesium chloride (1.0 M in THF, 2.7 mL, 2.70 mmol, 3.0 equiv) was added. The reaction mixture was allowed to warm to room temperature overnight. The next morning, the reaction mixture was poured into EtOAc (50 mL). The organic layer was washed with saturated NaHCO₃ (2×50 mL). The combined aqueous layers were extracted with EtOAc (1×50 mL), and the combined organic layers were dried over Na₂SO₄, filtered and concentrated in vacuo. The resulting crude material was used in the next step without further purification.

LCMS (+ESI): calc. [M+H]+=260; found 260.

### Step 3: Synthesis of (R)-4-(1-phenyl-4,4-difluoropyrrolidin-2-yl)thiazol-2-amine (intermediate 1)

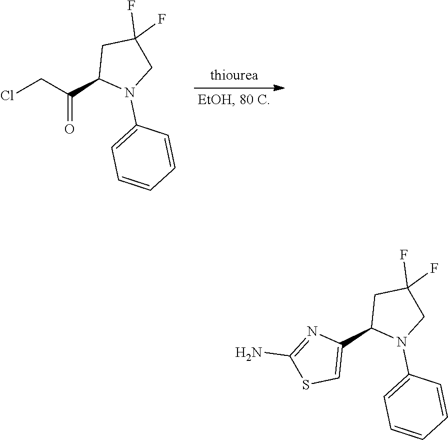 [SMILES: O=C(CCl)[C@H]1CC(F)(F)CN1c1ccccc1 ; Nc1nc([C@H]2CC(F)(F)CN2c2ccccc2)cs1]

A 20 mL vial with stir bar was charged with 2-(2-chloroacetyl)-4,4-difluoro-1-[phenyl]pyrrolidine (307 mg, 1.28 mmol, 1.0 equiv), thiourea (108 mg, 1.41 mmol, 1.1 equiv) and EtOH (7 mL). The resulting solution was stirred at 80° C. overnight. The next morning, the resulting solution was cooled and poured into EtOAc (100 ml). The mixture was washed with saturated NaHCO₃ (2×100 ml), and the combined aqueous layers were extracted with EtOAc (1×100 ml). The combined organic layers were dried over Na₂SO₄, filtered and concentrated in vacuo. The resulting crude material was purified via silica gel chromatography to yield the desired product.

LCMS (+ESI): calc. [M+H]+=282; found 282.

Intermediate 1 may be used in the preparation of compounds of the invention. For example, the following procedure describes the synthesis of a compound of the invention that contains an amide using intermediate 1 and a carboxylic acid.

Synthesis of amides using intermediate 1 and carboxylic acids Intermediate 1 is added to a solution of carboxylic acid and EDCI in DMF followed by DIPEA. After 24 hours the mixture was diluted with EtOAc, washed with water (4×), then the organic fraction was concentrated under reduced pressure and purified using silica gel chromatography.

### Synthesis of 2-(pyridin-4-yl)ethyl (R)-(4-(1-phenylpyrrolidin-2-yl)thiazol-2-yl)carbamate (Compound 1)

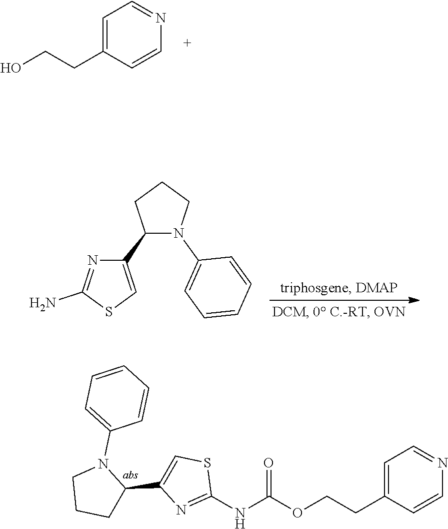 [SMILES: OCCc1ccncc1 ; Nc1nc([C@H]2CCCN2c2ccccc2)cs1 ; O=C(Nc1nc([C@H]2CCCN2c2ccccc2)cs1)OCCc1ccncc1]

To a solution of 2-(pyridin-4-yl)ethan-1-ol (29 mg, 0.24 mmol) and triphosgene (35 mg, 0.12 mmol) in DCM (2 mL) was added a solution of DMAP (99 mg, 0.84 mmol) in DCM (1 mL) dropwise at OoC. The reaction mixture was stirred at 0° C. for 30 mins, then (R)-4-(1-phenylpyrrolidin-2-yl)thiazol-2-amine (51 mg, 0.24 mmol) was added. The reaction mixture was warmed to RT and stirred at RT overnight. After completion, the reaction mixture was poured into water (10 mL) and extracted with DCM (10 mL×3). The combined organic layers were washed with brine (10 mL), dried over Na₂SO₄ and concentrated under reduced pressure. The crude product was purified by prep-TLC (DCM/MeOH=10/1, v/v) to provide the title compound (15 mg, 16% yield) as a pink solid.

LCMS (Agilent-S12): Rt=2.65 min; m/z [M+H]+ 395.2.

1H NMR (400 MHz, DMSO-d6) δ 11.71 (s, 1H), 8.49-8.47 (m, 2H), 7.34-7.32 (m, 2H), 7.09 (t, J=8.4 Hz, 2H), 6.66 (s, 1H), 6.55 (t, J=7.2 Hz, 1H), 6.47 (d, J=8.0 Hz, 2H), 4.69 (d, J=7.6 Hz, 1H), 4.40 (t, J=6.4 Hz, 2H), 3.56 (t, J=8.0 Hz, 1H), 3.26-3.16 (m, 1H), 2.97 (t, J=6.4 Hz, 2H), 2.24-2.17 (m, 1H), 1.99-1.92 (m, 3H).

### Synthesis of amide (R)-(4-(4,4-difluoro-1-phenylpyrrolidin-2-yl)thiazol-2-yl) 3-(4-pyridyloxy)propenamide (Compound 35)

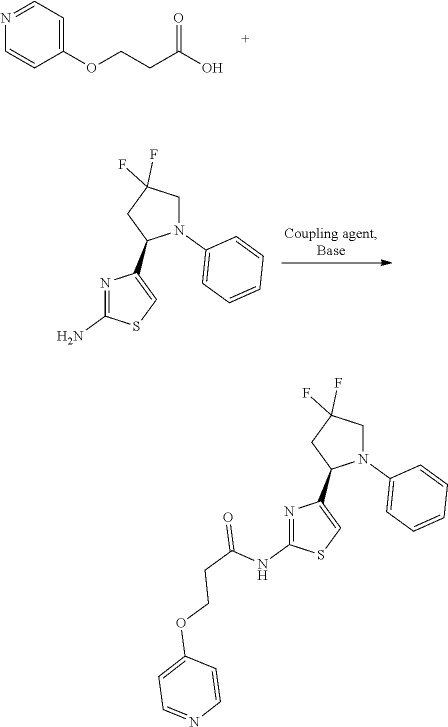 [SMILES: O=C(O)CCOc1ccncc1 ; Nc1nc([C@H]2CC(F)(F)CN2c2ccccc2)cs1 ; O=C(CCOc1ccncc1)Nc1nc([C@H]2CC(F)(F)CN2c2ccccc2)cs1]

A vial with stir bar is charged with 3-(4-pyridyloxy)propionic acid (1.00 equiv), 2-(2-aminothiazolyl)-4,4-difluoro-1-[phenyl]pyrrolidine (1.30 equiv), NMI (3.50 equiv) and ACN. TCFH (1.21 equiv) is added, and the vial is capped and placed in a 25° C. bath. The reaction mixture is stirred at 25° C. overnight. The next morning, the reaction mixture is poured into EtOAc and washed with brine. The combined aqueous layers are extracted with EtOAc, and the combined organic layers are dried over Na₂SO₄, filtered and concentrated in vacuo. The resulting crude material was purified via silica gel chromatography & Prep-HPLC or RP column to yield the desired product.

### Synthesis of 3-((4-pyridyl)methoxy)pyridinyl (R)-(4-(4,4-difluoro-1-phenylpyrrolidin-2-yl)thiazol-2-yl)amine (Compound 36)

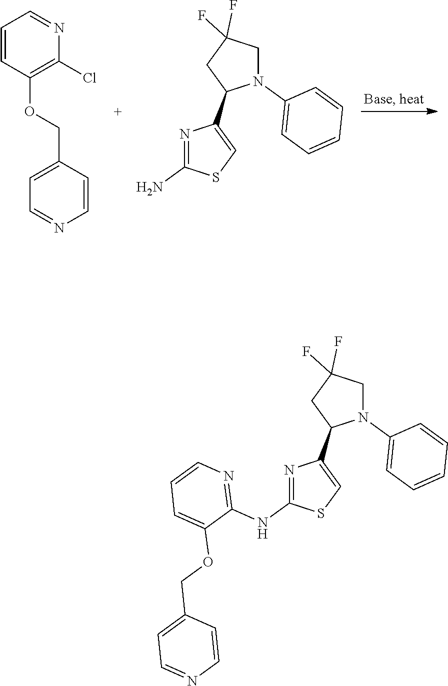 [SMILES: Clc1ncccc1OCc1ccncc1 ; Nc1nc([C@H]2CC(F)(F)CN2c2ccccc2)cs1 ; FC1(F)C[C@H](c2csc(Nc3ncccc3OCc3ccncc3)n2)N(c2ccccc2)C1]

A vial with stir bar is charged with 2-chloro-3-((4-pyridyl)methoxy)pyridine (1.00 equiv), (R)-4-(1-phenyl-4,4-difluoropyrrolidin-2-yl)thiazol-2-amine (1.30 equiv), Cesium carbonate (3.50 equiv) and DMF. The vial was capped and placed in a 100° C. bath. The reaction mixture was stirred at 100° C. overnight. The next morning, the reaction mixture was poured into EtOAc and washed with brine. The combined aqueous layers were extracted with EtOAc, and the combined organic layers were dried over Na₂SO₄, filtered and concentrated in vacuo. The resulting crude material was purified via silica gel chromatography & Prep-HPLC or RP column to yield the desired product.

### Synthesis of Compound 2

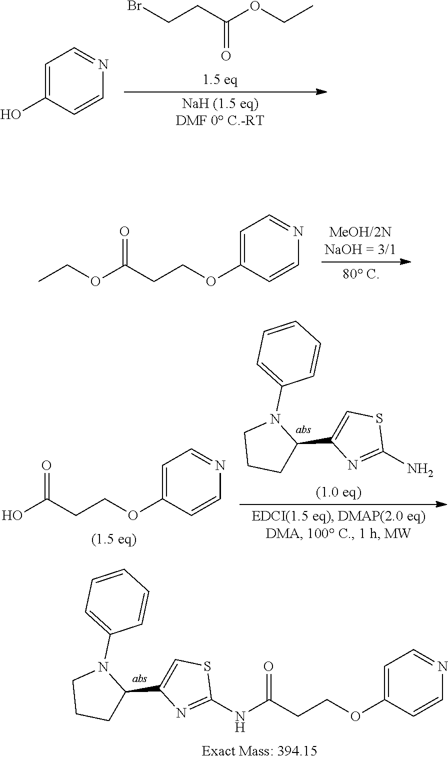

### Step 1: Synthesis of ethyl 3-(pyridin-4-yloxy)propanoate

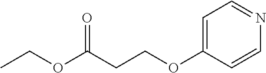

To a solution of pyridin-4-ol (500 mg, 5.26 mmol, 1 eq) in DMF (8 mL) was added NaH (316 g, 7.89 mmol, 1.5 eq) at 0° C. The solution was stirred at 0° C. for 0.5 h. ethyl 3-bromopropanoate (1.42 g, 7.89 mmol, 1.5 eq) was added to the solution. After stirring at room temperature for 4 h. The solution was extracted with EtOAc (10 mL×3), washed with water (10 mL×3), brine (10 mL), dried over Na₂SO₄ and concentrated to give a crude, the crude was purification by silica gel column (PE/EtOAc=2:1, v/v) to give the desired product (650 mg, 3.33 mmol, 63%) as a white oil.

LCMS (Waters-QDa-02): Rt 1.01 min; [M+1]⁺=196.09

### Step 2: Synthesis of 3-(pyridin-4-yloxy)propanoic acid

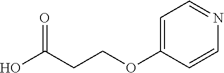 [SMILES: O=C(O)CCOc1ccncc1]

To a solution of ethyl 3-(pyridin-4-yloxy)propanoate (650 mg, 3.33 mmol, 1 eq) in THE (9 mL) and H₂ (3 mL) was added LiOH (240 mg, 9.99 mmol, 3 eq). After stirring at room temperature for 2 h, the reaction mixture was adjusted PH to 2 by HCl aqueous solution (1 M) and concentrated to give the crude (550 mg) as a yellow solid.

LCMS (Waters-QDa-02): Rt 0.27 min; [M+1]⁺=168.05

### Step 3: Synthesis of (R)—N-(4-(1-phenylpyrrolidin-2-yl)thiazol-2-yl)-3-(pyridin-4-yloxy)propanamide

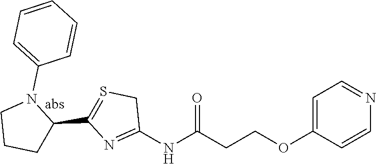

To a solution of 3-(pyridin-4-yloxy)propanoic acid (380 mg, 2.27 mmol, 2 eq) in DMA (3 mL) were added (R)-4-(1-phenylpyrrolidin-2-yl)thiazol-2-amine (100 mg, 1.13 mmol, 1 eq), EDCI (117 mg, 0.61 mmol, 1.5 eq) and DMAP (100 mg, 0.81 mmol, 2 eq). After stirring at 100° C. for 1 h in microwave. The solution was extracted with EtOAc (3 mL×3), washed with water (5 mL×3), brine (5 mL), dried over Na₂SO₄ and concentrated to give a crude, the crude was purification by prep.TLC (DCM/MeOH=10:1, v/v) to give the desired product (6 mg, 0.015 mmol, 1%) as a white solid.

LCMS (Agilent-1290): Rt: 2.61 min, m/z [M+1]+=395.1/396.2

HNMR (400 MHz, DMSO-d6): δ ppm: 12.23 (s, 1H), 7.93 (s, 2H), 7.72 (s, 1H), 7.19 (s, 1H), 7.13-7.06 (m, 2H), 6.73 (s, 1H), 6.56-6.52 (m, 1H), 6.47-6.45 (d, J=8 Hz, 1H), 6.41 (s, 1H), 4.74-4.72 (m, 1H), 4.28 (s, 2H), 3.58-3.54 (m, 2H), 2.98-2.94 (m, 2H), 2.26-2.19 (m, 1H), 2.01-1.93 (m, 3H).

HPLC (Agilent-1200-A2): Rt: 10.47 min, 96% purity.

### Synthesis of Compound 3

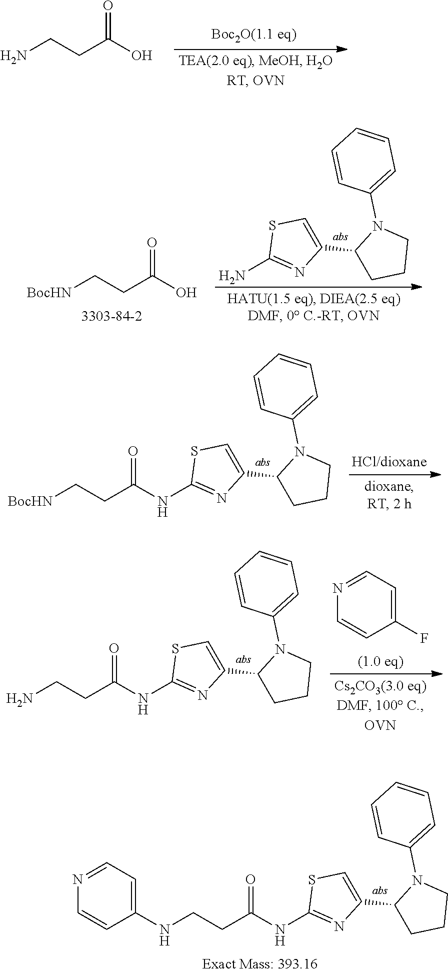

### Step 1: Synthesis of 3-((tert-butoxycarbonyl)amino)propanoic acid

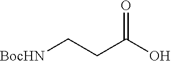 [SMILES: CC(C)(C)OC(=O)NCCC(=O)O]

To a solution of 3-aminopropanoic acid (1 g, 11.23 mmol, 1 eq) in MeOH (15 mL) were added Boc₂O (2.7 g, 12.35 mmol, 1.1 eq) and TEA (2.3 g, 22.46 mmol, 2.0 eq). After stirring at room temperature for 16 h, the reaction adjusted PH to 2 by HCl aqueous solution (1M) and extracted with DCM (30 mL×3), washed with water (20 mL×3), brine (20 mL), dried over Na₂SO₄ and concentrated to give the desired product (2.11 g, 11.23 mmol, 99%) as a white solid.

HNMR: (400 MHz, DMSO-d₆) δ ppm: 6.77 (s, 1H), 3.13-3.08 (m, 2H), 2.35-2.31 (m, 2H), 1.36 (s, 9H).

### Step 2: Synthesis of tert-butyl (R)-(3-oxo-3-((4-(1-phenylpyrrolidin-2-yl)thiazol-2-yl)amino)propyl)carbamate

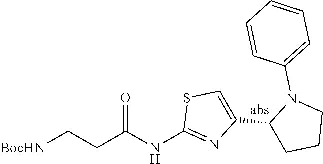 [SMILES: CC(C)(C)OC(=O)NCCC(=O)Nc1nc([C@H]2CCCN2c2ccccc2)cs1]

To a solution of 3-((tert-butoxycarbonyl)amino)propanoic acid (300 m, 1.58 mmol, 2 eq) in DMF (6 mL) were added (R)-4-(1-phenylpyrrolidin-2-yl)thiazol-2-amine (200 mg, 0.79 mmol, 1 eq), DIEA (369 mg, 1.58 mmol, 2 eq) and HATU (621 mg, 1.63 mmol, 2 eq) at 0° C. After stirring at room temperature for 16 h. The solution was extracted with EtOAc (6 mL×3), washed with water (6 mL×3), brine (6 mL), dried over Na₂SO₄ and concentrated to give a crude. The crude was purified by silica gel column (PE/EtOAc=2/1, v/v) to give the desired product (300 g, 0.72 mmol, 45%) as a white oil.

LCMS (Waters-QDa-02): Rt 1.89 min; [M+1]⁺=417.19/418.34

### Step 3: (R)-3-amino-n-(4-(1-phenylpyrrolidin-2-yl)thiazol-2-yl)propanamide

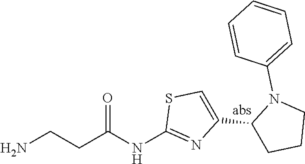 [SMILES: NCCC(=O)Nc1nc([C@H]2CCCN2c2ccccc2)cs1]

To a solution of tert-butyl (R)-(3-oxo-3-((4-(1-phenylpyrrolidin-2-yl)thiazol-2-yl)amino)propyl)carbamate (280 mg, 0.67 mmol, 1 eq) in dioxane (3 mL) was added HCl/dioxanne (4 M) (6 ml). After stirring at room temperature for 2 h, the reaction mixture was concentrated under reduced pressure to give the desired product (210 mg, 0.67 mmol, 99%) as a yellow oil.

LCMS (Waters-QDa-02): Rt 0.67 min; [M+1]⁺=317.19/418.20

### Step 4: Synthesis of (R)—N-(4-(1-phenylpyrrolidin-2-yl)thiazol-2-yl)-3-(pyridin-4-ylamino)propanamide

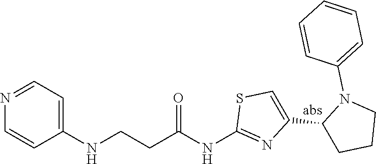 [SMILES: O=C(CCNc1ccncc1)Nc1nc([C@H]2CCCN2c2ccccc2)cs1]

To a solution of (R)-3-amino-N-(4-(1-phenylpyrrolidin-2-yl)thiazol-2-yl)propanamide (300 mg, 0.94 mmol, 1 eq) in DMF (5 mL) at were added 4-fluoropyridine (125 mg, 0.94 mmol, 1 eq) and Cs2CO3 (924 mg, 2.85 mmol, 3 eq). The solution was stirred at 100° C. for 16 h. The reaction was extracted with ethyl acetate (6 mL×3), washed with water (6 mL×3), brine (6 mL), dried over Na₂SO₄ and concentrated to give a crude, the crude was purified by prep.TLC (DCM/MeOH=10/1, v/v) to give the desired product (80 mg, 0.94 mmol, 21%) as a yellow solid.

LCMS (SHIMADZU-2020-P2): Rt: 3.45 min, m/z [M+1]+=394.2/385.1

HNMR (400 MHz, DMSO-d6): δ ppm: 11.99 (s, 1H), 8.01-8.00 (d, J=4 Hz, 2H), 7.10-7.08 (m, 2H), 7.06 (s, 1H), 6.70-6.46 (m, 6H), 4.75-4.72 (m, 1H), 3.60-3.55 (m, 1H), 3.41-3.36 (m, 2H), 3.25-3.17 (m, 1H), 2.69-2.65 (m, 2H), 2.27-2.22 (m, 1H), 2.02-1.92 (m, 3H).

HPLC (Agilent-1200-A2): Rt: 10.40 min, 98% purity.

### Synthesis of Compound 4

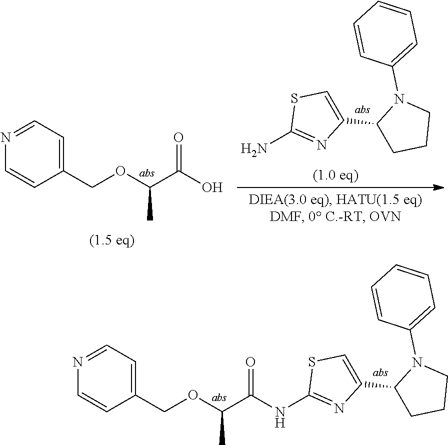 [SMILES: C[C@@H](OCc1ccncc1)C(=O)O ; Nc1nc([C@H]2CCCN2c2ccccc2)cs1 ; C[C@@H](OCc1ccncc1)C(=O)Nc1nc([C@H]2CCCN2c2ccccc2)cs1]

Step 1: Synthesis of (R)—N-(4-((R)-1-phenylpyrrolidin-2-yl)thiazol-2-yl)-2-(pyridin-4-ylmethoxy)propanamide

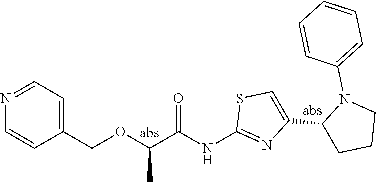 [SMILES: C[C@@H](OCc1ccncc1)C(=O)Nc1nc([C@H]2CCCN2c2ccccc2)cs1]

To a solution of (S)-2-(pyridin-4-ylmethoxy)propanoic acid (210 mg, 1.16 mmol, 2 eq) in DMA (3 mL) were added (R)-4-(1-phenylpyrrolidin-2-yl)thiazol-2-amine (142 mg, 0.58 mmol, 1 eq), EDCI (111 mg, 0.57 mmol, 1.5 eq) and DMAP (141 mg, 1.16 mmol, 2 eq). After stirring at 130° C. for 5 h in microwave. The solution was extracted with EtOAc (3 mL×3), washed with water (5 mL×3), brine (5 mL), dried over Na₂SO₄ and concentrated to give a crude, the crude was purification by prep.TLC (DCM/MeOH=10:1, v/v) to give the desired product (18 mg, 0.04 mmol, 7%) as a white solid.

LCMS (Agilent-1290): Rt: 2.74 min, m/z [M+1]+=409.2/410.2

HNMR (400 MHz, DMSO-d6): δ ppm: 12.18 (s, 1H), 8.53-8.51 (m, 2H), 7.37-7.36 (m, 2H), 7.10-7.07 (m, 2H), 6.76 (s, 1H), 6.57-6.53 (m, 1H), 6.49-6.47 (m, 2H), 4.76-4.74 (m, 1H), 4.62-4.58 (m, 1H), 4.52-4.47 (m, 1H), 4.29-4.24 (m, 1H), 3.60-3.56 (m, 1H), 3.27-3.23 (m, 1H), 2.29-2.20 (m, 1H), 2.04-1.98 (m, 3H), 1.40-1.38 (m, 3H).

HPLC (Agilent-1200-A2): Rt: 10.77 min, 98% purity.

### Synthesis of Compound 5

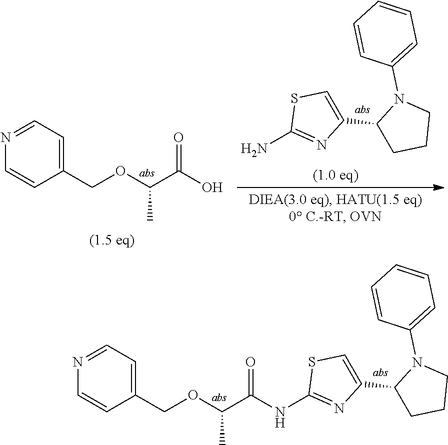 [SMILES: C[C@H](OCc1ccncc1)C(=O)O ; Nc1nc([C@H]2CCCN2c2ccccc2)cs1 ; C[C@H](OCc1ccncc1)C(=O)Nc1nc([C@H]2CCCN2c2ccccc2)cs1]

### Step 1: Synthesis of (S)—N-(4-((R)-1-phenylpyrrolidin-2-yl)thiazol-2-yl)-2-(pyridin-4-ylmethoxy)propanamide

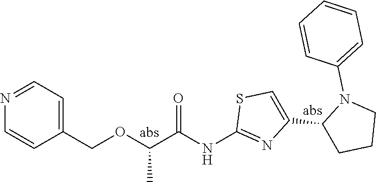 [SMILES: C[C@H](OCc1ccncc1)C(=O)Nc1nc([C@H]2CCCN2c2ccccc2)cs1]

To a solution of (S)-2-(pyridin-4-ylmethoxy)propanoic acid (50 mg, 0.3 mmol, 1.5 eq) in DMA (2 mL) were added (R)-4-(1-phenylpyrrolidin-2-yl)thiazol-2-amine (50 mg, 0.25 mmol, 1 eq), EDCI (58 mg, 0.3 mmol, 1.5 eq) and DMAP (49 mg, 0.4 mmol, 2 eq). After stirring at 100° C. for 2 h in microwave. The solution was extracted with EtOAc (3 mL×3), washed with water (5 mL×3), brine (5 mL), dried over Na₂SO₄ and concentrated to give a crude, the crude was purification by prep.TLC (DCM/MeOH=10:1, v/v) to give the desired product (20 mg, 0.04 mmol, 4%) as a white solid.

LCMS (Agilent-1290): Rt: 2.77 min, m/z [M+1]+=409.2/410.2

HNMR (400 MHz, DMSO-d6): δ ppm: 12.18 (s, 1H), 8.54-8.52 (m, 2H), 7.37-7.36 (m, 2H), 7.11-7.07 (m, 2H), 6.76 (s, 1H), 6.57-6.53 (m, 1H), 6.49-6.47 (m, 2H), 4.76-4.74 (d, J=8 Hz, 1H), 4.63-4.58 (m, 1H), 4.52-4.48 (m, 1H), 4.47-4.24 (m, 1H), 3.60-3.56 (m, 1H), 3.26-3.16 (m, 1H), 2.27-2.20 (m, 1H), 2.11-1.95 (m, 3H), 1.40-1.37 (m, 3H).

HPLC (Agilent-1200-A2): Rt: 10.71 min, 98% purity.

### Synthesis of Compound 6

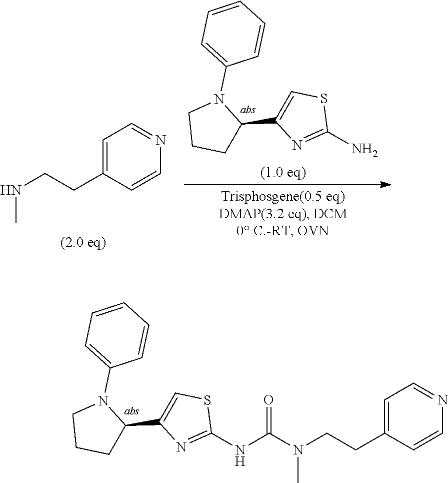

### Step 1: Synthesis of (R)-1-methyl-3-(4-(1-phenylpyrrolidin-2-yl)thiazol-2-yl)-1-(2-(pyridin-4-yl)ethyl)urea

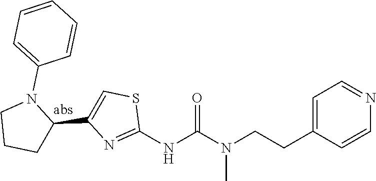 [SMILES: CN(CCc1ccncc1)C(=O)Nc1nc([C@H]2CCCN2c2ccccc2)cs1]

To a solution of (R)-4-(1-phenylpyrrolidin-2-yl)thiazol-2-amine (100 mg, 0.4 mmol, 1 eq) in DCM (4 mL) were added Triphosgene (60 mg, 0.2 mmol, 0.5 eq), DMAP (159 mg, 1.3 mmol, 3.2 eq) at 0° C. The solution was stirred at 0° C. for 2 h. N-methyl-2-(pyridin-4-yl)ethan-1-amine (111 mg, 0.81 mmol, 2 eq) was added to the solution. After stirring at room temperature for 16 h. The solution was extracted with DCM (5 mL×3), washed with water (5 mL×3), brine (5 mL), dried over Na₂SO₄ and concentrated to give a crude, the crude was purification by prep.TLC (DCM/MeOH=20:1, v/v) to give the desired product (56 mg, 0.13 mmol, 33%) as a white solid.

LCMS (Agilent): Rt: 2.53 min, m/z [M+1]⁺=408.4/409.3

HNMR (400 MHz, DMSO-d6): δ ppm: 10.71 (s, 1H), 8.46-8.44 (m, 2.5H), 7.30-7.28 (m, 2H), 7.21-7.20 (m, 0.5H), 7.11-7.07 (m, 2H), 6.57-6.53 (m, 1H), 6.49-6.46 (m, 3H), 4.68-4.66 (d, J=8 Hz, 1H), 3.62-3.56 (m, 3H), 3.27-3.22 (m, 1H), 2.93 (s, 3H), 2.84-2.80 (m, 2H), 2.24-2.17 (m, 1H), 2.02-1.95 (m, 3H).

HPLC (Agilent-1200-A2): Rt: 9.51 min, 97% purity.

### Synthesis of Compound 7

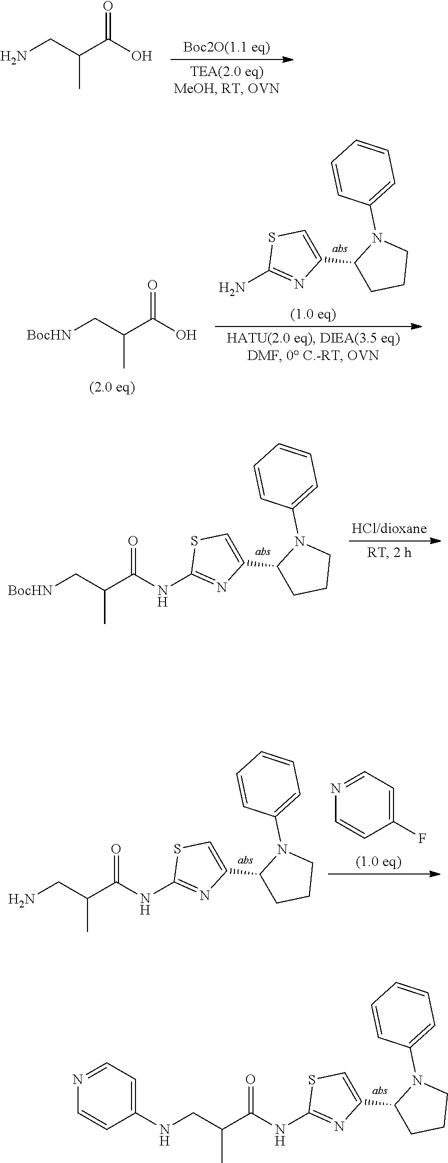

### Step 1: Synthesis of 3-((tert-butoxycarbonyl)amino)-2-methylpropanoic acid

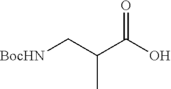

To a solution of 3-amino-2-methylpropanoic acid (500 mg, 4.85 mmol, 1 eq) in MeOH (25 mL) were added Boc₂O (1.16 g, 5.33 mmol, 1.1 eq) and TEA (982 mg, 9.7 mmol, 2.0 eq). After stirring at room temperature for 16 h, the reaction adjusted PH to 2 by HCl aqueous solution (1M) and extracted with DCM (30 mL×3), washed with water (20 mL×3), brine (20 mL), dried over Na₂SO₄ and concentrated to give the desired product (900 mg, 4.85 mmol, 91%) as a white solid.

HNMR (400 MHz, DMSO-d6): δ ppm: 6.80 (s, 1H), 3.18-3.08 (m, 1H), 2.94-2.88 (m, 1H), 2.46-2.41 (m, 1H), 1.36 (s, 9H), 1.01-0.99 (d, J=8 Hz, 3H).

### Step 2: Synthesis of tert-butyl (2-methyl-3-oxo-3-((4-((R)-1-phenylpyrrolidin-2-yl)thiazol-2-yl)amino)propyl)carbamate

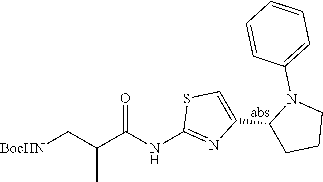 [SMILES: CC(CNC(=O)OC(C)(C)C)C(=O)Nc1nc([C@H]2CCCN2c2ccccc2)cs1]

To a solution of 3-((tert-butoxycarbonyl)amino)-2-methylpropanoic acid (414 mg, 2.03 mmol, 2.5 eq) in DMF (6 mL) were added (R)-4-(1-phenylpyrrolidin-2-yl)thiazol-2-amine (200 mg, 0.82 mmol, 1 eq), DIEA (369 mg, 2.04 mmol, 2.5 eq) and HATU (369 mg, 2.86 mmol, 3.5 eq) at 0° C. After stirring at room temperature for 16 h. The solution was extracted with EtOAc (6 mL×3), washed with water (6 mL×3), brine (6 mL), dried over Na₂SO₄ and concentrated to give a crude. The crude was purified by silica gel column (PE/EtOAc=2/1, v/v) to give the desired product (280 mg, 0.82 mmol, 80%) as a white solid.

LCMS (Waters-QDa-02): Rt 1.98 min; [M+1]⁺=431.1/432.2

### Step 3: Synthesis of 3-amino-2-methyl-N-(4-((R)-1-phenylpyrrolidin-2-yl)thiazol-2-yl)propanamide

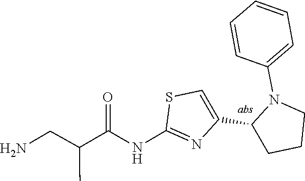 [SMILES: CC(CN)C(=O)Nc1nc([C@H]2CCCN2c2ccccc2)cs1]

To a solution of tert-butyl (2-methyl-3-oxo-3-((4-((R)-1-phenylpyrrolidin-2-yl)thiazol-2-yl)amino)propyl)carbamate (140 mg, 0.32 mmol, 1 eq) in dioxane (2 mL) was added HCl/dioxanne (4 M) (4 ml). After stirring at room temperature for 2 h, the reaction mixture was concentrated under reduced pressure to give the desired product (170 mg crude) as a yellow oil.

LCMS (Waters-QDa-02): Rt 0.93 min; [M+1]⁺=331.1/332.2

### Step 4: Synthesis of 2-methyl-N-(4-((R)-1-phenylpyrrolidin-2-yl)thiazol-2-yl)-3-(pyridin-4-ylamino)propanamide

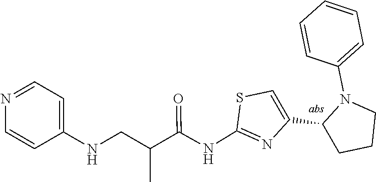 [SMILES: CC(CNc1ccncc1)C(=O)Nc1nc([C@H]2CCCN2c2ccccc2)cs1]

To a solution of 3-amino-2-methyl-N-(4-((R)-1-phenylpyrrolidin-2-yl)thiazol-2-yl)propanamide (50 mg, 0.15 mmol, 1 eq) in DMF (5 mL) were added 4-fluoropyridine (21 mg, 0.15 mmol, 1 eq) and Cs₂CO₃ (155 mg, 0.45 mmol, 3 eq). The solution was stirred at 100° C. for 16 h. The reaction was extracted with ethyl acetate (6 mL×3), washed with water (6 mL×3), brine (6 mL), dried over Na₂SO₄ and concentrated to give a crude, the crude was purified by prep.TLC (DCM/MeOH=10/1, v/v) to give the desired product (7 mg, 0.15 mmol, 11%) as a yellow solid.

LCMS (Agilent-1290): Rt: 2.65 min, m/z [M+1]⁺=408.2/409.3

HNMR (400 MHz, DMSO-d6): δ ppm: 12.14 (s, 1H), 8.00-7.98 (m, 2H), 7.10-7.16 (m, 2H), 6.71-6.46 (m, 7H), 4.74-4.72 (d, J=8 Hz, 1H), 3.57-3.55 (m, 1H), 3.24-3.16 (m, 2H), 3.09 (m, 1H), 2.94-2.90 (m, 1H), 2.24-2.21 (m, 1H), 1.97-1.93 (m, 3H), 1.14-1.12 (m, 3H).

HPLC (Agilent-1200-A2): Rt: 13.86 min, 100% purity.

### Synthesis of Compound 8

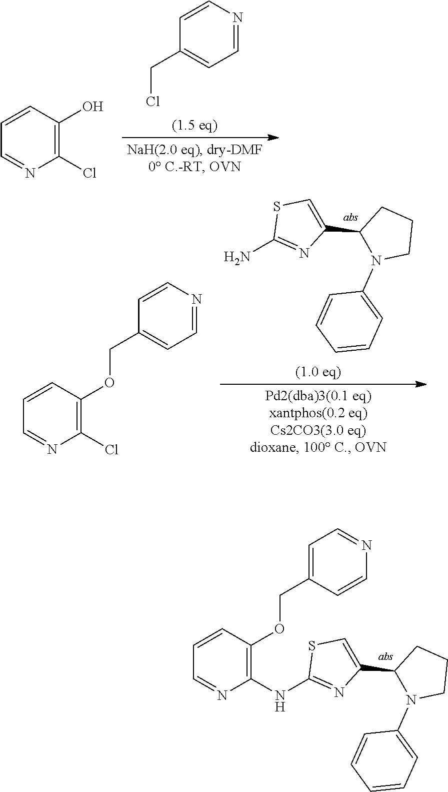 [SMILES: ClCc1ccncc1 ; Oc1cccnc1Cl ; Clc1ncccc1OCc1ccncc1 ; Nc1nc([C@H]2CCCN2c2ccccc2)cs1 ; c1ccc(N2CCC[C@@H]2c2csc(Nc3ncccc3OCc3ccncc3)n2)cc1]

### Step 1: Synthesis of 2-chloro-3-(pyridin-4-ylmethoxy)pyridine

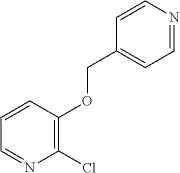 [SMILES: Clc1ncccc1OCc1ccncc1]

To a mixture of 2-chloropyridin-3-ol (500 mg, 3.8 mmol, 1 eq) in DMF (15 mL) was added NaH (310 mg, 7.7 mmol, 2 eq) at 0° C. After stirring at 0° C. for 0.5 h, 4-(chloromethyl)pyridine (947 mg, 5.8 mmol, 1.5 eq) was added to the solution. The solution was stirred at room temperature for 16 h. The solution was quenched with NH₄Cl solution (20 ml), extracted with EtOAc (20 mL×3), washed with water (30 mL×3), brine (30 mL), dried over Na₂SO₄ and concentrated to give a crude, the residue was purified by silica gel column (PE/EtOAc=2/1, v/v) to give the desired product (50 mg, 0.22 mmol, 6%) as a yellow oil.

LCMS (Waters-QDa-02): Rt 1.34 min; [M+1]⁺=221.1/222.1

### Step 2: Synthesis of (R)-4-(1-phenylpyrrolidin-2-yl)-N-(3-(pyridin-4-ylmethoxy)pyridin-2-yl)thiazol-2-amine

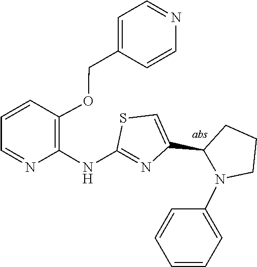 [SMILES: c1ccc(N2CCC[C@@H]2c2csc(Nc3ncccc3OCc3ccncc3)n2)cc1]

To a solution of (R)-4-(1-phenylpyrrolidin-2-yl)thiazol-2-amine (96 mg, 0.4 mmol, 1 eq) in dioxane (5 mL) were added 2-chloro-3-(pyridin-4-ylmethoxy)pyridine (86 mg, 0.4 mmol, 1 eq), Pd2(dba)₃ (40 mg, 0.03 mmol, 0.1 eq), xantphos (45 mg, 0.07 mmol, 0.2 eq) and Cs2CO3 (385 mg, 1.18 mmol, 3 eq). The solution was stirred at 100° C. for 16 h. The reaction was concentrated to give a crude, the crude was purified by prep.TLC (DCM/MeOH=10/1, v/v) to give the desired product (60 mg, 0.13 mmol, 35%) as a yellow solid.

LCMS (Agilent-1290): Rt: 3.24 min, m/z [M+1]+=430.2/432.2

HNMR (400 MHz, DMSO-d6): δ ppm: 10.29 (s, 1H), 8.60-8.59 (m, 2H), 7.87-7.86 (m, 1H), 7.62-7.61 (m, 2H), 7.37-7.35 (m, 1H), 7.11-7.07 (m, 2H), 6.91-6.89 (m, 1H), 6.57-6.48 (m, 4H), 5.31 (s, 2H), 4.74-4.72 (m, 1H), 3.60 (s, 1H), 3.25-3.16 (m, 1H), 2.24-2.22 (m, 3H).

HPLC (Agilent-1200-A2): Rt: 11.01 min, 98% purity.

### Synthesis of Compound 9

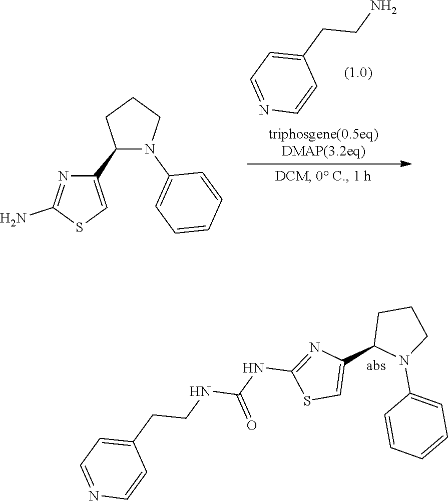 [SMILES: Nc1nc([C@H]2CCCN2c2ccccc2)cs1 ; NCCc1ccncc1 ; O=C(NCCc1ccncc1)Nc1nc([C@H]2CCCN2c2ccccc2)cs1]

### Step 1: Synthesis of (R)-1-(4-(1-phenylpyrrolidin-2-yl)thiazol-2-yl)-3-(2-(pyridin-4-yl)ethyl)urea

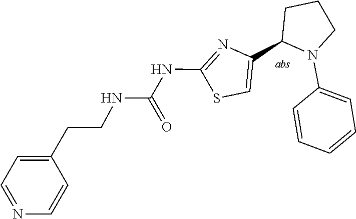 [SMILES: O=C(NCCc1ccncc1)Nc1nc([C@H]2CCCN2c2ccccc2)cs1]

To a solution of (R)-4-(1-phenylpyrrolidin-2-yl)thiazol-2-amine (100 mg, 0.4 mmol, 1 eq) in DCM (4 mL) were added Triphosgene (61 mg, 0.4 mmol, 0.5 eq), DMAP (160 mg, 1.3 mmol, 3.2 eq) at 0° C. The solution was stirred at 0° C. for 2 h. 2-(pyridin-4-yl)ethan-1-amine (47 mg, 0.4 mmol, 1 eq) was added to the solution. After stirring at room temperature for 16 h. The solution was extracted with DCM (5 mL×3), washed with water (5 mL×3), brine (5 mL), dried over Na₂SO₄ and concentrated to give a crude, the crude was purification by prep.TLC (DCM/MeOH=20:1, v/v) to give the desired product (10 mg, 0.02 mmol, 6%) as a white solid.

LCMS (Agilent-1290): Rt: 2.42 min, m/z [M+1]+=394.1/395.2

HNMR (400 MHz, DMSO-d6): δ ppm: 10.38 (s, 1H), 8.48-8.46 (m, 2H), 7.26-7.25 (m, 2H), 7.10-7.06 (m, 2H), 6.56-6.46 (m, 5H), 4.67-4.65 (m, 1H), 3.55-3.51 (m, 1H), 3.43-3.38 (m, 2H), 3.27-3.20 (m, 1H), 2.79-2.76 (m, 2H), 2.21-2.17 (m, 1H), 2.01-1.94 (m, 3H).

HPLC (Agilent-1200-A2): Rt: 9.91 min, 95% purity.

### Synthesis of Compound 10

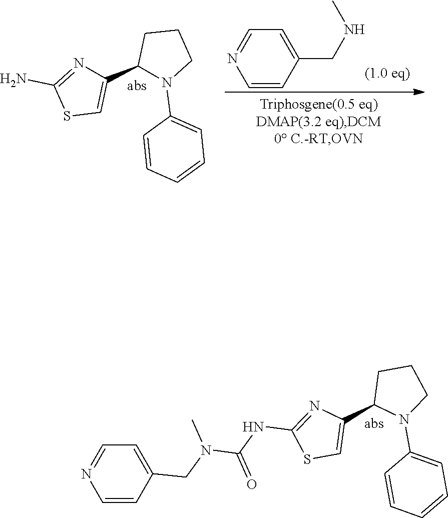

### Step 1: Synthesis of (R)-1-methyl-3-(4-(1-phenylpyrrolidin-2-yl)thiazol-2-yl)-1-(pyridin-4-ylmethyl)urea

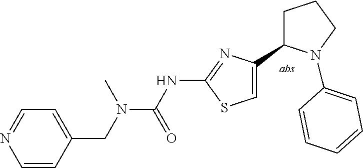 [SMILES: CN(Cc1ccncc1)C(=O)Nc1nc([C@H]2CCCN2c2ccccc2)cs1]

To a solution of (R)-4-(1-phenylpyrrolidin-2-yl)thiazol-2-amine (100 mg, 0.4 mmol, 1 eq) in DCM (4 mL) were added Triphosgene (61 mg, 0.2 mmol, 0.5 eq), DMAP (160 mg, 1.3 mmol, 3.2 eq) at 0° C. The solution was stirred at 0° C. for 2 h. N-methyl-1-(pyridin-4-yl)methanamine (50 mg, 0.4 mmol, 1 eq) was added to the solution. After stirring at room temperature for 16 h. The solution was extracted with DCM (5 mL×3), washed with water (5 mL×3), brine (5 mL), dried over Na₂SO₄ and concentrated to give a crude, the crude was purification by prep.TLC (DCM/MeOH=20:1, v/v) to give the desired product (40 mg, 0.1 mmol, 25%) as a white solid.

LCMS (Agilent-1290): Rt: 2.53 min, m/z [M+1]+=394.1/395.2

HNMR (400 MHz, DMSO-d6): δ ppm: 10.88 (s, 1H), 8.52-8.51 (m, 2H), 7.21-7.20 (m, 2H), 7.11-7.07 (m, 2H), 6.57-6.65 (m, 2H), 6.48-6.46 (m, 2H), 4.69-4.67 (d, J=8 Hz, 1H), 4.61 (s, 1H), 3.60-3.56 (m, 1H), 3.26-3.20 (m, 1H), 2.99 (s, 3H), 2.24-2.07 (m, 1H), 1.98-1.90 (m, 3H).

HPLC (Agilent-1200-A2): Rt: 9.82 min, 99% purity.

### Synthesis of Compound 11

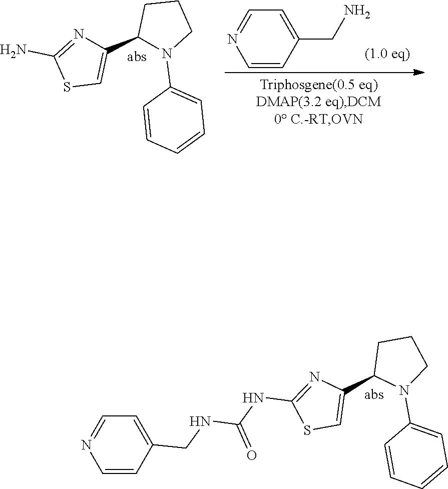

### Step 1: Synthesis of (R)-1-(4-(1-phenylpyrrolidin-2-yl)thiazol-2-yl)-3-(pyridin-4-ylmethyl)urea

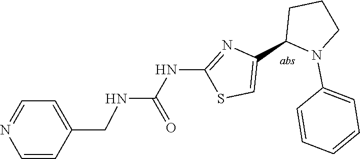 [SMILES: O=C(NCc1ccncc1)Nc1nc([C@H]2CCCN2c2ccccc2)cs1]

To a solution of (R)-4-(1-phenylpyrrolidin-2-yl)thiazol-2-amine (100 mg, 0.4 mmol, 1 eq) in DCM (4 mL) were added Triphosgene (61 mg, 0.2 mmol, 0.5 eq), DMAP (160 mg, 1.3 mmol, 3.2 eq) at 0° C. The solution was stirred at 0° C. for 2 h. pyridin-4-ylmethanamine (44 mg, 0.4 mmol, 1 eq) was added to the solution. After stirring at room temperature for 16 h. The solution was extracted with DCM (5 mL×3), washed with water (5 mL×3), brine (5 mL), dried over Na₂SO₄ and concentrated to give a crude, the crude was purification by prep.TLC (DCM/MeOH=20:1, v/v) to give the desired product (25 mg, 0.06 mmol, 16%) as a white solid.

LCMS (Agilent-1290): Rt: 2.45 min, m/z [M+1]+=380.1/381.2

HNMR (400 MHz, DMSO-d6): δ ppm: 10.68 (s, 1H), 8.50-8.49 (m, 2H), 7.27-7.25 (m, 2H), 7.10-7.06 (m, 3H), 6.56-6.47 (m, 4H), 4.70-4.68 (m, 1H), 4.36-4.35 (m, 2H), 3.56-3.52 (m, 1H), 3.27-3.21 (m, 1H), 2.23-2.19 (m, 1H), 1.97 (m, 3H).

HPLC (Agilent-1200-A2): Rt: 9.68 min, 98% purity.

### Synthesis of Compound 12

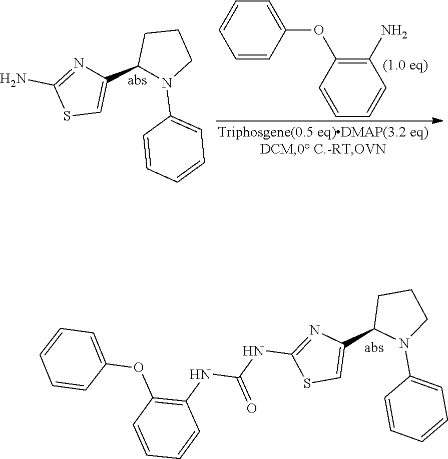

### Step 1: Synthesis of (R)-1-(2-phenoxyphenyl)-3-(4-(1-phenylpyrrolidin-2-yl)thiazol-2-yl)urea

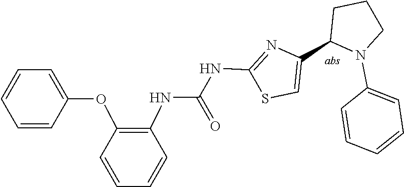 [SMILES: O=C(Nc1nc([C@H]2CCCN2c2ccccc2)cs1)Nc1ccccc1Oc1ccccc1]

To a solution of (R)-4-(1-phenylpyrrolidin-2-yl)thiazol-2-amine (100 mg, 0.4 mmol, 1 eq) in DCM (4 mL) were added Triphosgene (60 mg, 0.2 mmol, 0.5 eq), DMAP (159 mg, 1.3 mmol, 3.2 eq) at 0° C. The solution was stirred at 0° C. for 2 h. 2-phenoxyaniline (76 mg, 0.41 mmol, 1 eq) was added to the solution. After stirring at room temperature for 16 h. The solution was extracted with DCM (5 mL×3), washed with water (5 mL×3), brine (5 mL), dried over Na₂SO₄ and concentrated to give a crude, the crude was purification by prep.TLC (DCM/MeOH=20:1, v/v) to give the desired product (50 mg, 0.10 mmol, 26%) as a white solid.

LCMS (Agilent-1290): Rt: 4.44 min, m/z [M+1]+=457.3/458.2

HNMR (400 MHz, DMSO-d6): δ ppm: 11.00 (s, 1H), 8.92 (s, 1H), 8.25-8.23 (m, 1H), 7.42-7.40 (m, 2H), 7.38-7.00 (m, 7H), 6.91-6.88 (m, 1H), 6.61 (s, 1H), 6.56-6.52 (m, 1H), 6.48-6.46 (m, 2H), 4.67-4.65 (m, 1H), 3.55-3.51 (m, 1H), 3.31-3.20 (m, 1H), 2.18-2.11 (m, 1H), 2.04-1.91 (m, 3H).

HPLC (Agilent-1200-A2): Rt: 16.02 min, 98% purity.

### Synthesis of Compound 13

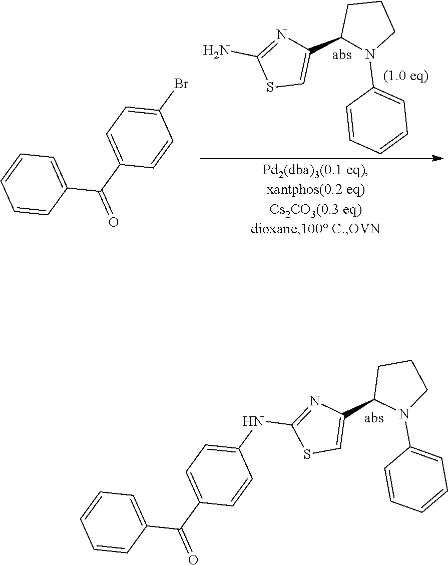 [SMILES: O=C(c1ccccc1)c1ccc(Br)cc1 ; Nc1nc([C@H]2CCCN2c2ccccc2)cs1 ; O=C(c1ccccc1)c1ccc(Nc2nc([C@H]3CCCN3c3ccccc3)cs2)cc1]

### Step 1: Synthesis of (R)-phenyl(4-((4-(1-phenylpyrrolidin-2-yl)thiazol-2-yl)amino)phenyl)methanone

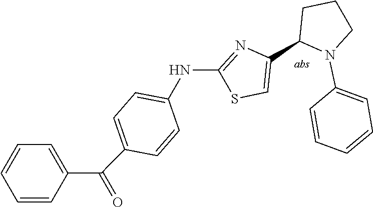 [SMILES: O=C(c1ccccc1)c1ccc(Nc2nc([C@H]3CCCN3c3ccccc3)cs2)cc1]

To a solution of (4-bromophenyl)(phenyl)methanone (106 mg, 0.4 mmol, 1 eq) in dioxane (6 mL) were added (R)-4-(1-phenylpyrrolidin-2-yl)thiazol-2-amine (100 mg, 0.4 mmol, 1 eq), Pd2(dba)₃ (42 mg, 0.04 mmol, 0.1 eq), xantphos (47.2 mg, 0.08 mmol, 0.2 eq) and Cs₂CO₃ (398 mg, 1.22 mmol, 3 eq). The solution was stirred at 100° C. for 16 h. The reaction was concentrated to give a crude, the crude was purified by prep.TLC (DCM/MeOH=10/1, v/v) to give the desired product (72 mg, 0.16 mmol, 41%) as a yellow solid.

LCMS (Agilet-1290): Rt: 4.43 min, m/z [M+1]+=426.1/427.2

HNMR (400 MHz, DMSO-d6): δ ppm: 10.67 (s, 1H), 7.78-7.73 (m, 4H), 7.71-7.51 (m, 5H), 7.12-7.08 (m, 2H), 6.57-6.52 (m, 4H), 4.77-4.75 (m, 1H), 3.60-3.55 (m, 1H), 3.29-3.23 (m, 1H), 2.27-2.20 (m, 1H), 2.15-1.98 (m, 3H).

HPLC (Agilent-1200-A2): Rt: 15.73 min, 96% purity.

### Synthesis of Compound 14

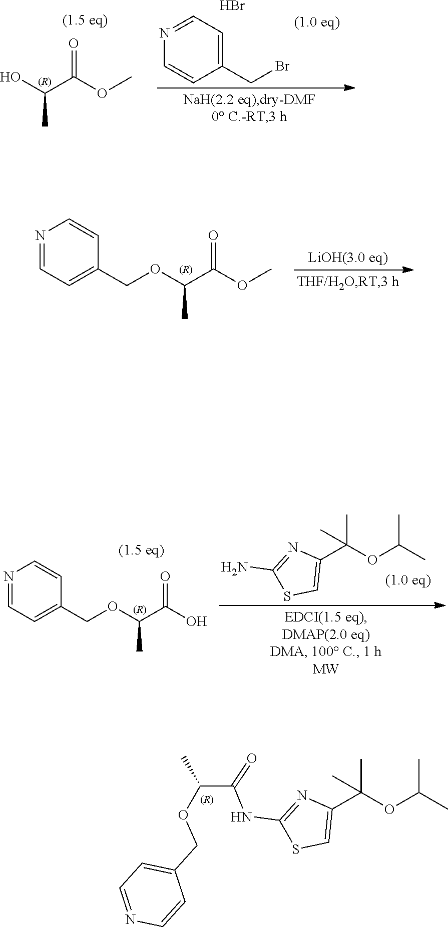

### Step 1: Synthesis of methyl (R)-2-(pyridin-4-ylmethoxy)propanoate

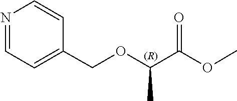

To a solution of methyl (R)-2-hydroxypropanoate (1 g, 9.6 mmol, 1.5 eq) in DMF (20 mL) was added NaH (0.56 g, 14 mmol, 2.2 eq) at 0° C. After stirring at 0° C. for 0.5 h, 4-(bromomethyl)pyridine (1.6 g, 6.3 mmol, 1 eq) was added to the solution. The solution was stirred at room temperature for 2 h. The solution was quenched with NH4Cl solution (20 ml), extracted with EtOAc (20 mL×3), washed with water (30 mL×3), brine (30 mL), dried over Na₂SO₄ and concentrated to give a crude, the residue was purified by silica gel column (PE/EtOAc=2/1, v/v) to give the desired product (300 mg, 9.6 mmol, 24%) as a yellow oil.

LCMS (Waters-QDa-02): Rt 1.35 min; [M+1]⁺=196.1

### Step 2: Synthesis of (R)-2-(pyridin-4-ylmethoxy)propanoic acid

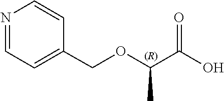

To a solution of methyl (R)-2-(pyridin-4-ylmethoxy)propanoate (300 mg, 1.53 mmol, 1 eq) in THE (3 mL) and H₂ (1 mL) was added LiOH (111 mg, 4.61 mmol, 3 eq). After stirring at room temperature for 2 h, the reaction mixture was adjusted PH to 2 by HCl aqueous solution (1 M) and concentrated to give the crude (270 mg) as a yellow solid.

LCMS (Waters-QDa-02): Rt 0.94 min; [M+1]⁺=182.08

### Step 3: Synthesis of (R)—N-(4-(2-isopropoxypropan-2-yl)thiazol-2-yl)-2-(pyridin-4-ylmethoxy)propanamide

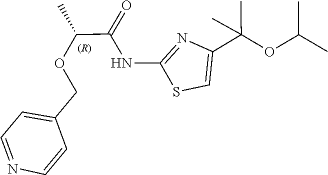

To a solution of (R)-2-(pyridin-4-ylmethoxy)propanoic acid (280 mg, 1.4 mmol, 1.5 eq) in DMA (3 mL) were added 4-(2-isopropoxypropan-2-yl)thiazol-2-amine (100 mg, 0.93 mmol, 1 eq), EDCI (144 mg, 0.75 mmol, 1.5 eq) and DMAP (122 mg, 1 mmol, 2 eq). After stirring at 100° C. for 3 h in microwave. The solution was extracted with EtOAc (3 mL×3), washed with water (5 mL×3), brine (5 mL), dried over Na₂SO₄ and concentrated to give a crude, the crude was purification by prep.TLC (DCM/MeOH=10:1, v/v) to give the desired product (60 mg, 0.93 mmol, 33%) as a white solid.

LCMS (Agilent-1290): Rt: 2.26 min, m/z [M+1]+=364.1

HNMR (400 MHz, DMSO-d6): δ ppm: 12.29 (s, 1H), 8.53-8.52 (m, 2H), 7.37-7.35 (m, 2H), 7.05 (s, 1H), 4.62-4.49 (m, 2H), 4.30-4.25 (m, 1H), 3.63-3.57 (m, 1H), 1.46 (s, 6H), 1.40-1.38 (m, 3H), 0.93-0.91 (m, 6H).

HPLC (Agilent-1200-A2): Rt: 9.48 min, 100% purity.

### Synthesis of Compound 15

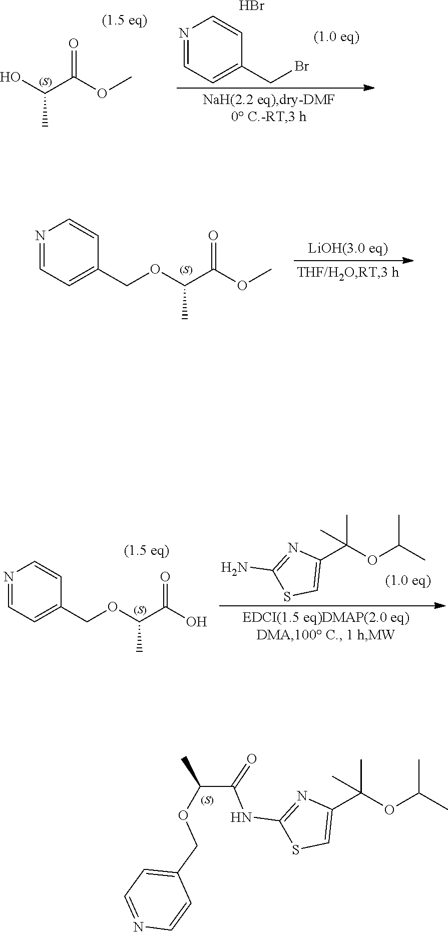

### Step 1: Synthesis of methyl (S)-2-(pyridin-4-ylmethoxy)propanoate

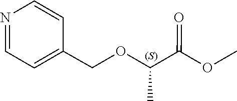

To a solution of methyl (S)-2-hydroxypropanoate (1 g, 9.6 mmol, 1 eq) in DMF (20 mL) was added NaH (0.56 g, 14 mmol, 2.2 eq) at 0° C. After stirring at 0° C. for 0.5 h, 4-(bromomethyl)pyridine (1.6 g, 6.3 mmol, 1 eq) was added to the solution. The solution was stirred at room temperature for 2 h. The solution was quenched with NH4Cl solution (20 ml), extracted with EtOAc (20 mL×3), washed with water (30 mL×3), brine (30 mL), dried over Na₂SO₄ and concentrated to give a crude, the residue was purified by silica gel column (PE/EtOAc=2/1, v/v) to give the desired product (160 mg, 9.6 mmol, 8%) as a yellow oil.

LCMS (Waters-QDa-02): Rt 1.36 min; [M+1]+=196.08

### Step 2: Synthesis of (S)-2-(pyridin-4-ylmethoxy)propanoic acid

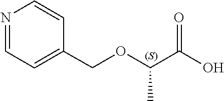

To a solution of methyl (S)-2-(pyridin-4-ylmethoxy)propanoate (160 mg, 0.82 mmol, 1 eq) in THF (3 mL) and H₂O (1 mL) was added LiOH (58 mg, 2.46 mmol, 3 eq). After stirring at room temperature for 2 h, the reaction mixture was adjusted PH to 2 by HCl aqueous solution (1 M) and concentrated to give the crude (140 mg) as a yellow solid.

LCMS (Waters-QDa-02): Rt 0.91 min; [M+1]³⁰=182.08

### Step 3: Synthesis of (S)—N-(4-(2-isopropoxypropan-2-yl)thiazol-2-yl)-2-(pyridin-4-ylmethoxy)propanamide

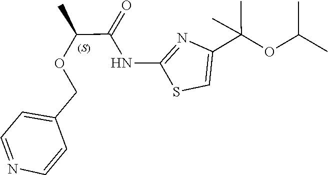

To a solution of (S)-2-(pyridin-4-ylmethoxy)propanoic acid (67 mg, 0.37 mmol, 1.5 eq) in DMA (2 mL) were added 4-(2-isopropoxypropan-2-yl)thiazol-2-amine (50 mg, 0.25 mmol, 1 eq), EDCI (71 mg, 0.37 mmol, 1.5 eq) and DMAP (61 mg, 0.5 mmol, 2 eq). After stirring at 100° C. for 1 h in microwave. The solution was extracted with EtOAc (3 mL×3), washed with water (5 mL×3), brine (5 mL), dried over Na₂SO₄ and concentrated to give a crude, the crude was purification by prep.TLC (DCM/MeOH=10:1, v/v) to give the desired product (8.5 mg, 0.25 mmol, 8%) as a white solid.

LCMS (Agilent-1290): Rt: 2.25 min, m/z [M+1]+=364.1

HNMR (400 MHz, DMSO-d6): δ ppm: 12.28 (s, 1H), 8.53-8.51 (m, 2H), 7.37-7.35 (m, 2H), 7.05 (s, 1H), 4.62-4.49 (m, 2H), 4.30-4.25 (m, 1H), 3.63-3.57 (m, 1H), 1.46 (s, 6H), 1.40-1.38 (m, 3H), 0.93-0.91 (m, 6H).

HPLC (Agilent-1200-A2): Rt: 9.44 min, 100% purity.

### Synthesis of Compound 16

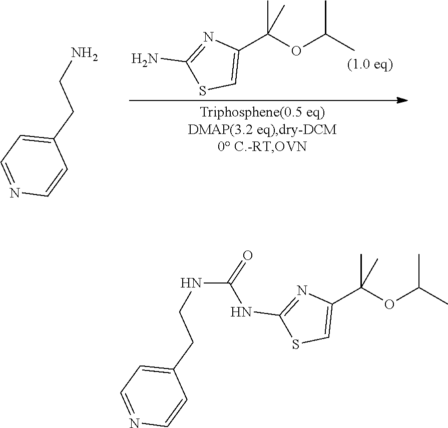 [SMILES: NCCc1ccncc1 ; CC(C)OC(C)(C)c1csc(N)n1 ; CC(C)OC(C)(C)c1csc(NC(=O)NCCc2ccncc2)n1]

### Step 1: Synthesis of 1-(4-(2-isopropoxypropan-2-yl)thiazol-2-yl)-3-(2-(pyridin-4-yl)ethyl)urea

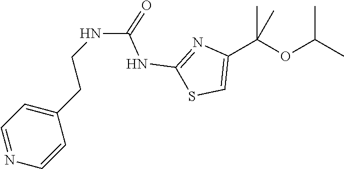 [SMILES: CC(C)OC(C)(C)c1csc(NC(=O)NCCc2ccncc2)n1]

To a solution of 4-(2-isopropoxypropan-2-yl)thiazol-2-amine (50 mg, 0.24 mmol, 1 eq) in DCM (4 mL) were added Triphosgene (37 mg, 0.12 mmol, 0.5 eq), DMAP (97 mg, 0.79 mmol, 3.2 eq) at 0° C. The solution was stirred at 0° C. for 2 h. 2-(pyridin-4-yl)ethan-1-amine (31 mg, 0.24 mmol, 1 eq) was added to the solution. After stirring at room temperature for 16 h. The solution was extracted with DCM (5 mL×3), washed with water (5 mL×3), brine (5 mL), dried over Na₂SO₄ and concentrated to give a crude, the crude was purification by prep.TLC (DCM/MeOH=20:1, v/v) to give the desired product (20 mg, 0.057 mmol, 22%) as a white solid.

LCMS (Agilent-1290): Rt: 1.93 min, m/z [M+1]+=349.2

HNMR (400 MHz, DMSO-d6): δ ppm: 10.54 (s, 1H), 8.48-8.45 (m, 2H), 7.26-7.25 (m, 2H), 6.82 (s, 1H), 6.41 (s, 1H), 3.62-3.56 (m, 1H), 3.43-3.38 (m, 2H), 2.80-2.76 (m, 2H), 1.41 (s, 6H), 0.90-0.89 (m, 6H).

HPLC (Agilent-1200-A2): Rt: 8.49 min, 98% purity.

### Synthesis of Compound 17

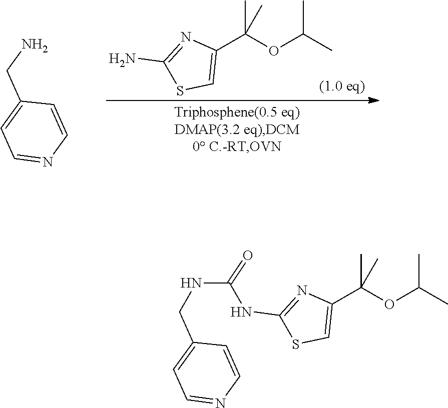 [SMILES: NCc1ccncc1 ; CC(C)OC(C)(C)c1csc(N)n1 ; CC(C)OC(C)(C)c1csc(NC(=O)NCc2ccncc2)n1]

### Step 1: Synthesis of 1-(4-(2-isopropoxypropan-2-yl)thiazol-2-yl)-3-(pyridin-4-ylmethyl)urea

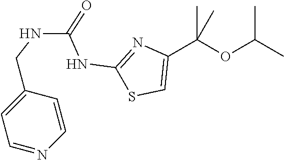 [SMILES: CC(C)OC(C)(C)c1csc(NC(=O)NCc2ccncc2)n1]

To a solution of 4-(2-isopropoxypropan-2-yl)thiazol-2-amine (200 mg, 0.99 mmol, 1 eq) in DCM (6 mL) were added Triphosgene (148 mg, 0.49 mmol, 0.5 eq), DMAP (390 mg, 3.19 mmol, 3.2 eq) at 0° C. The solution was stirred at 0° C. for 2 h. pyridin-4-ylmethanamine (108 mg, 0.99 mmol, 1 eq) was added to the solution. After stirring at room temperature for 16 h. The solution was extracted with DCM (5 mL×3), washed with water (5 mL×3), brine (5 mL), dried over Na₂SO₄ and concentrated to give a crude, the crude was purification by prep.TLC (DCM/MeOH=20:1, v/v) to give the desired product (185 mg, 0.99 mmol, 55%) as a white solid.

LCMS (Agilent-1290): Rt: 1.97 min, m/z [M+1]+=335.2/336.2

HNMR (400 MHz, DMSO-d6): δ ppm: 10.83 (s, 1H), 8.50-8.49 (m, 2H), 7.27-7.26 (m, 2H), 7.00 (s, 1H), 6.83 (s, 1H), 4.37-4.35 (m, 2H), 3.63-3.57 (m, 1H), 1.43 (s, 6H), 0.92-0.90 (m, 6H).

HPLC (Agilent-1200-A2): Rt: 8.41 min, 100% purity.

### Synthesis of Compound 18

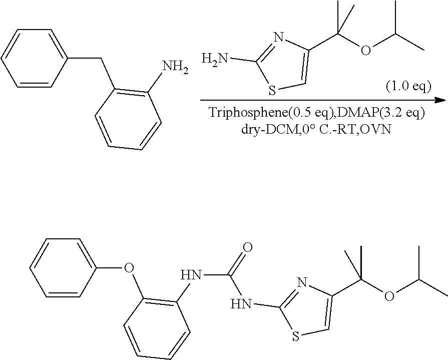 [SMILES: Nc1ccccc1Cc1ccccc1 ; CC(C)OC(C)(C)c1csc(N)n1 ; CC(C)OC(C)(C)c1csc(NC(=O)Nc2ccccc2Oc2ccccc2)n1]

### Step 1: Synthesis of 1-(4-(2-isopropoxypropan-2-yl)thiazol-2-yl)-3-(2-phenoxyphenyl)urea

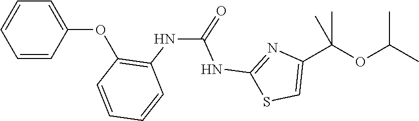 [SMILES: CC(C)OC(C)(C)c1csc(NC(=O)Nc2ccccc2Oc2ccccc2)n1]

To a solution of 2-phenoxyaniline (46 mg, 0.24 mmol, 1 eq) in DCM (4 mL) were added Triphosgene (37 mg, 0.12 mmol, 0.5 eq), DMAP (97 mg, 0.79 mmol, 3.2 eq) at 0° C. The solution was stirred at 0° C. for 2 h. 4-(2-isopropoxypropan-2-yl)thiazol-2-amine (50 mg, 0.24 mmol, 1 eq) was added to the solution. After stirring at room temperature for 16 h. The solution was extracted with DCM (5 mL×3), washed with water (5 mL×3), brine (5 mL), dried over Na₂SO₄ and concentrated to give a crude, the crude was purification by prep.TLC (DCM/MeOH=20:1, v/v) to give the desired product (20 mg, 0.048 mmol, 19%) as a white solid.

LCMS (Agilent-1290): Rt: 4.18 min, m/z [M+1]+=412.2

HNMR (400 MHz, DMSO-d6): δ ppm: 11.14 (s, 1H), 8.80 (s, 1H), 8.26-8.23 (m, 1H), 7.42-7.37 (m, 2H), 7.18-7.12 (m, 2H), 7.05-7.00 (m, 3H), 6.99-6.90 (m, 2H), 3.63-3.57 (m, 1H), 1.42 (s, 6H), 0.90-0.89 (m, 6H).

HPLC (Agilent-1200-A2): Rt: 9.70 min, 100% purity.

### Synthesis of Compound 19

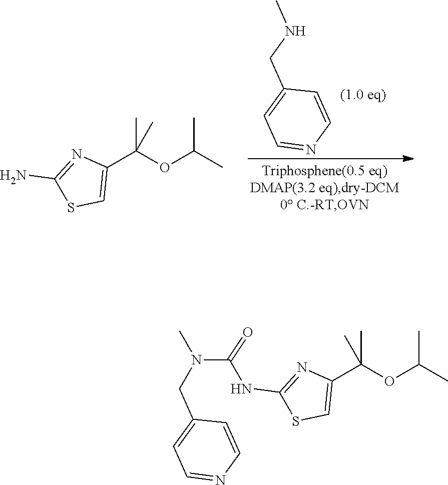

### Step 1: 3-(4-(2-isopropoxypropan-2-yl)thiazol-2-yl)-1-methyl-1-(pyridin-4-ylmethyl)urea

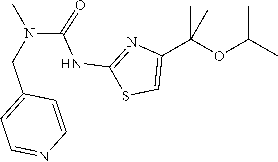 [SMILES: CC(C)OC(C)(C)c1csc(NC(=O)N(C)Cc2ccncc2)n1]

To a solution of 4-(2-isopropoxypropan-2-yl)thiazol-2-amine (50 mg, 0.24 mmol, 1 eq) in DCM (4 mL) were added Triphosgene (37 mg, 0.12 mmol, 0.5 eq), DMAP (97 mg, 0.79 mmol, 3.2 eq) at 0° C. The solution was stirred at 0° C. for 2 h. N-methyl-1-(pyridin-4-yl)methanamine (30 mg, 0.24 mmol, 1 eq) was added to the solution. After stirring at room temperature for 16 h. The solution was extracted with DCM (5 mL×3), washed with water (5 mL×3), brine (5 mL), dried over Na₂SO₄ and concentrated to give a crude, the crude was purification by prep.TLC (DCM/MeOH=20:1, v/v) to give the desired product (40 mg, 0.11 mmol, 45%) as a white solid.

LCMS (Agilent-1290): Rt: 2.05 min, m/z [M+1]+=349.2/350.2

HNMR (400 MHz, DMSO-d6): δ ppm: 10.91 (s, 1H), 8.52-8.51 (s, 2H), 7.22-7.20 (s, 2H), 6.86 (s, 1H), 4.62 (s, 2H), 3.62-3.56 (m, 1H), 3.29 (s, 3H), 1.45 (s, 6H), 0.92-0.90 (m, 6H).

HPLC (Agilent-1200-A2): Rt: 8.42 min, 99% purity.

### Synthesis of Compound 20

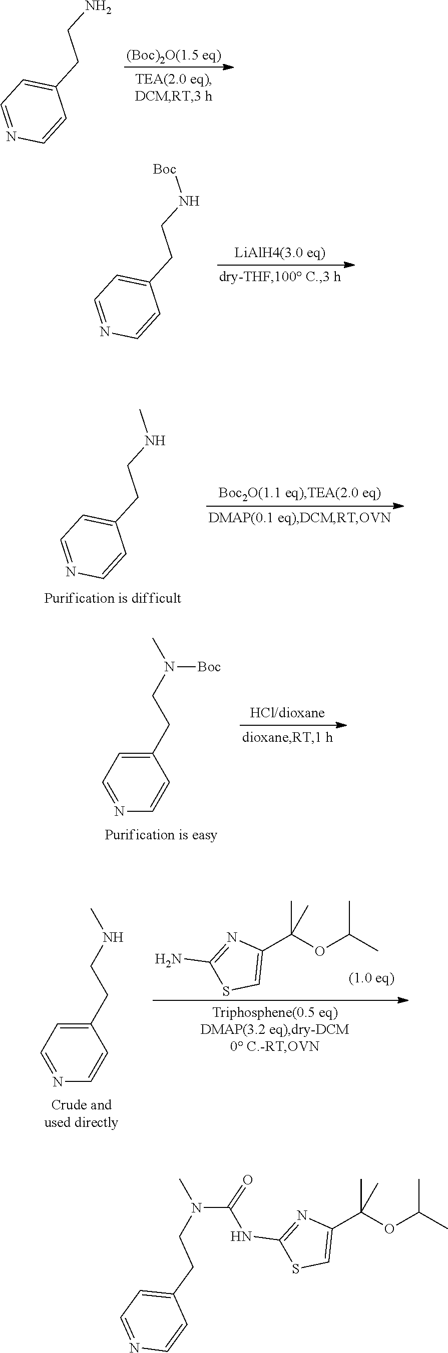

### Step 1: Synthesis of tert-butyl (2-(pyridin-4-yl)ethyl)carbamate

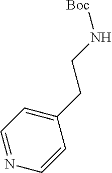 [SMILES: CC(C)(C)OC(=O)NCCc1ccncc1]

To a solution of 2-(pyridin-4-yl)ethan-1-amine (400 mg, 3.27 mmol, 1 eq) in DCM (5 mL) were added Boc₂O (1.07 g, 4.91 mmol, 1.5 eq) and TEA (663 mg, 6.55 mmol, 2 eq). After stirring at room temperature for 16 h, the reaction mixture was concentrated under reduced pressure. The residue was purified by silica gel column (DCM/MeOH=50/1, v/v) to give a mixture of desired product (700 mg, 3.27 mmol, 96%) as a white oil.

LCMS (Waters-QDa-02): Rt 2.90 min; [M+1]⁺=223.2

### Step 2: Synthesis of N-methyl-2-(pyridin-4-yl)ethan-1-amine

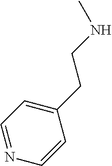

To a solution of tert-butyl (2-(pyridin-4-yl)ethyl)carbamate (100 mg, 0.45 mmol, 1 eq) in THE (3 mL) was added LiAlH4 (51 mg, 1.35 mmol, 3 eq). After stirring at 100° C. for 3 h, the reaction mixture was quenched with water (1 mL) and added diatomite to filtered with (DCM/MeOH=10:1) ammonia. The mixture was concentrated to give the crude (70 mg, crude) as a white solid.

LCMS (Waters-QDa-02): Rt 0.34 min; [M+1]⁺=136.9

### Step 3: Synthesis of tert-butyl methyl(2-(pyridin-4-yl)ethyl)carbamate

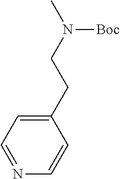 [SMILES: CN(CCc1ccncc1)C(=O)OC(C)(C)C]

To a mixture of N-methyl-2-(pyridin-4-yl)ethan-1-amine (280 mg, 2.05 mmol, 1 eq) in DCM (5 mL) were added Boc2 O (493 mg, 2.26 mmol, 1.1 eq), TEA (416 mg, 4.1 mmol, 2.0 eq) and DMAP (25 mg, 0.2 mmol, 0.1 eq). After stirring at room temperature for 16 h, the reaction mixture was concentrated under reduced pressure to give a crude, the crude was purification by prep.TLC (DCM/MeOH=20:1, v/v) to give the desired product (88 mg, 2.05 mmol, 16%) as a white solid.

LCMS (Waters-QDa-02): Rt 0.55 min; [M+1]⁺=236.7

### Step 4: Synthesis of N-methyl-2-(pyridin-4-yl)ethan-1-amine

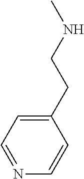

To a solution of tert-butyl methyl(2-(pyridin-4-yl)ethyl)carbamate (88 g, 0.37 mmol, 1 eq) in dioxane (1 mL) was added HCl/dioxane (2 mL). The reaction concentrated to give the desired product (80 mg crude).

LCMS (Waters-QDa-02): Rt 0.34 min; [M+1]+=136.9

### Step 5: Synthesis of 3-(4-(2-isopropoxypropan-2-yl)thiazol-2-yl)-1-methyl-1-(2-(pyridin-4-yl)ethyl)urea

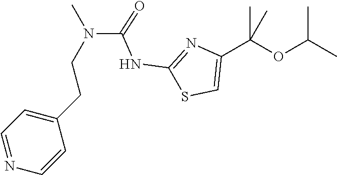 [SMILES: CC(C)OC(C)(C)c1csc(NC(=O)N(C)CCc2ccncc2)n1]

To a solution of N-methyl-2-(pyridin-4-yl)ethan-1-amine (80 mg crude, 0.58 mmol, 1.5 eq) in DCM (5 mL) were added Triphosgene (68 mg, 0.22 mmol, 0.5 eq) and DMAP (270 mg, 1.47 mmol, 3.2 eq). The reaction mixture was stirred at 0° C. for 2 h. 4-(2-isopropoxypropan-2-yl)thiazol-2-amine (80 mg, 0.58 mmol, 1 eq) was added to the solution. The reaction mixture was stirred at room temperature for 16 h. The solution was concentrated to give a crude, the crude was purification by prep.TLC (DCM/MeOH=10:1, v/v) to give the desired product (60 mg, 0.58 mmol, 36%) as a white solid.

LCMS (Agilent-1290): Rt: 1.95 min, m/z [M+1]+=363.2

HNMR (400 MHz, DMSO-d6): δ ppm: 10.69 (s, 1H), 8.45-8.44 (s, 2H), 7.30-7.28 (m, 2H), 6.83 (s, 1H), 3.62-3.56 (m, 3H), 2.94 (s, 3H), 2.84-2.80 (m, 2H), 1.45 (s, 6H), 0.91-0.90 (m, 6H).

HPLC (Agilent-1200-A2): Rt: 8.54 min, 99% purity.

### Synthesis of Compound 21

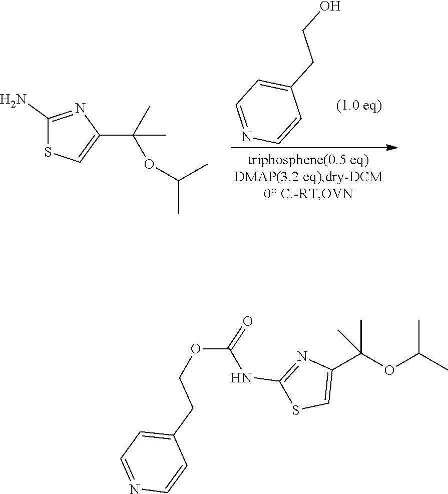 [SMILES: CC(C)OC(C)(C)c1csc(N)n1 ; OCCc1ccncc1 ; CC(C)OC(C)(C)c1csc(NC(=O)OCCc2ccncc2)n1]

### Step 1: Synthesis of 2-(pyridin-4-yl)ethyl (4-(2-isopropoxypropan-2-yl)thiazol-2-yl)carbamate

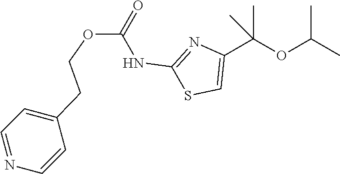 [SMILES: CC(C)OC(C)(C)c1csc(NC(=O)OCCc2ccncc2)n1]

To a solution of 4-(2-isopropoxypropan-2-yl)thiazol-2-amine (100 mg, 0.49 mmol, 1 eq) in DCM (5 mL) were added Triphosgene (74 mg, 0.24 mmol, 0.5 eq), DMAP (195 mg, 1.59 mmol, 3.2 eq) at 0° C. The solution was stirred at 0° C. for 2 h. 2-(pyridin-4-yl)ethan-1-ol (61 mg, 0.49 mmol, 1 eq) was added to the solution. After stirring at room temperature for 16 h. The solution was extracted with DCM (3 mL×3), washed with water (5 mL×3), brine (5 mL), dried over Na₂SO₄ and concentrated to give a crude, the crude was purification by prep.TLC (DCM/MeOH=20:1, v/v) to give the desired product (100 mg, 0.49 mmol, 57%) as a white solid.

LCMS (Agilent-1290): Rt: 2.15 min, m/z [M+1]+=350.1

HNMR (400 MHz, DMSO-d6): δ ppm: 11.77 (s, 1H), 8.48-8.46 (m, 2H), 7.34-7.32 (m, 2H), 6.98 (s, 1H), 4.39-4.35 (m, 2H), 3.61-3.53 (m, 1H), 2.7-2.94 (m, 2H), 1.43 (s, 6H), 0.90-0.89 (m, 6H).

HPLC (Agilent-1200-A2): Rt: 8.65 min, 96% purity.

### Synthesis of Compound 22

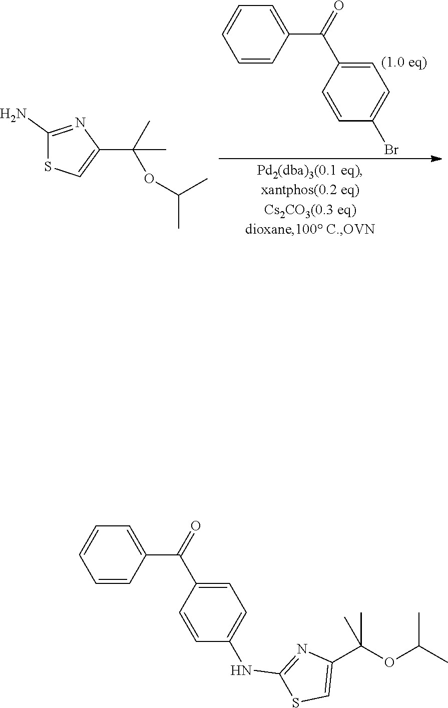 [SMILES: CC(C)OC(C)(C)c1csc(N)n1 ; O=C(c1ccccc1)c1ccc(Br)cc1 ; CC(C)OC(C)(C)c1csc(Nc2ccc(C(=O)c3ccccc3)cc2)n1]

### Step 1: Synthesis of (4-((4-(2-isopropoxypropan-2-yl)thiazol-2-yl)amino)phenyl)(phenyl)methanone

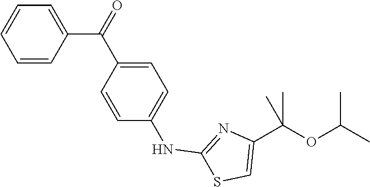 [SMILES: CC(C)OC(C)(C)c1csc(Nc2ccc(C(=O)c3ccccc3)cc2)n1]

To a solution of 4-(2-isopropoxypropan-2-yl)thiazol-2-amine (50 mg, 0.25 mmol, 1 eq) in dioxane (5 mL) were added (4-bromophenyl)(phenyl)methanone (65 mg, 0.25 mmol, 1 eq), Pd2(dba)₃ (26 mg, 0.025 mmol, 0.1 eq), xantphos (29 mg, 0.05 mmol, 0.2 eq) and Cs₂CO₃ (244 mg, 0.75 mmol, 3 eq). The solution was stirred at 100° C. for 16 h. The reaction was concentrated to give a crude, the crude was purified by prep.TLC (DCM/MeOH=10/1, v/v) to give the desired product (15 mg, 0.03 mmol, 15%) as a yellow solid.

LCMS (Agilent-1290): Rt: 4.13 min, m/z [M+1]+=381.1

HNMR (400 MHz, DMSO-d6): δ ppm: 10.66 (s, 1H), 7.80-7.52 (m, 9H), 6.85 (s, 1H), 3.72-3.63 (m, 1H), 1.49 (s, 6H), 0.95-0.94 (m, 6H).

HPLC (Agilent-1200-A2): Rt: 8.64 min, 100% purity.

### Synthesis of Compound 23

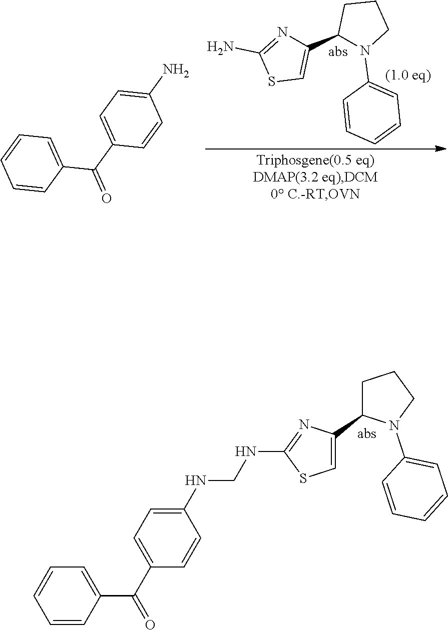 [SMILES: Nc1ccc(C(=O)c2ccccc2)cc1 ; Nc1nc([C@H]2CCCN2c2ccccc2)cs1 ; O=C(c1ccccc1)c1ccc(NCNc2nc([C@H]3CCCN3c3ccccc3)cs2)cc1]

### Step 1: Synthesis of (R)-1-(4-benzoylphenyl)-3-(4-(1-phenylpyrrolidin-2-yl)thiazol-2-yl)urea

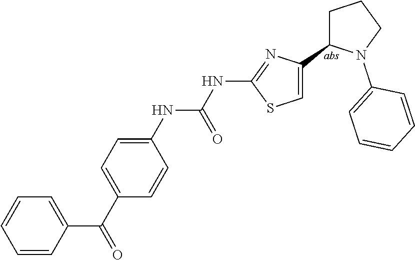 [SMILES: O=C(Nc1ccc(C(=O)c2ccccc2)cc1)Nc1nc([C@H]2CCCN2c2ccccc2)cs1]

To a solution of (R)-4-(1-phenylpyrrolidin-2-yl)thiazol-2-amine (100 mg, 0.4 mmol, 1 eq) in DCM (3 mL) were added Triphosgene (60 mg, 0.2 mmol, 0.5 eq), DMAP (159 mg, 1.3 mmol, 3.2 eq) at 0° C. The solution was stirred at 0° C. for 2 h. (4-aminophenyl)(phenyl)methanone (80 mg, 0.4 mmol, 1 eq) was added to the solution. After stirring at room temperature for 16 h. The solution was extracted with DCM (5 mL×3), washed with water (5 mL×3), brine (5 mL), dried over Na₂SO₄ and concentrated to give a crude, the crude was purification by prep.TLC (DCM/MeOH=20:1, v/v) to give the desired product (26 mg, 0.055 mmol, 13%) as a white solid.

LCMS (Agilent-1290): Rt: 4.20 min, m/z [M+1]+=469.2/470.2

HNMR (400 MHz, DMSO-d6): δ ppm: 10.64 (s, 1H), 9.34 (s, 1H), 7.76-7.63 (m, 8H), 7.57-7.53 (m, 2H), 7.12-7.08 (m, 2H), 6.66 (s, 1H), 6.57-6.54 (m, 1H), 6.51-6.49 (m, 2H), 4.74-4.72 (m, 1H), 3.59-3.55 (m, 1H), 3.27-3.23 (m, 1H), 2.26-2.22 (m, 1H), 2.09-1.97 (m, 3H).

HPLC (Agilent-1200-A2): Rt: 14.93 min, 95% purity.

### Synthesis of Compound 24

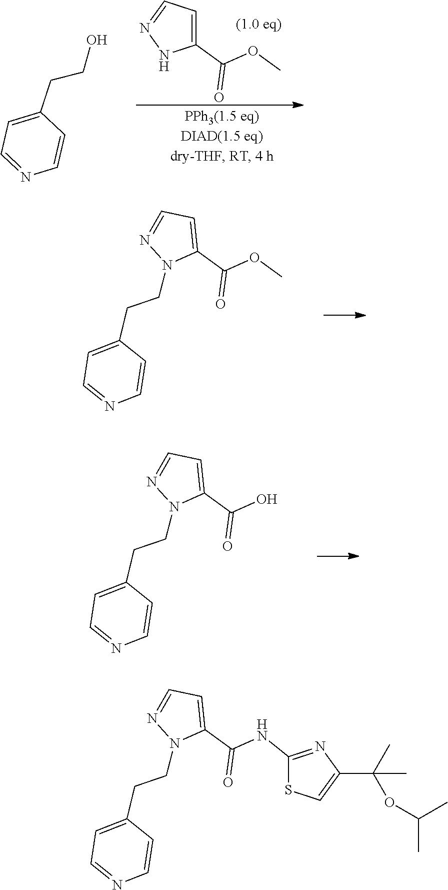

### Step 1: Synthesis of methyl 1-(2-(pyridin-4-yl)ethyl)-1H-pyrazole-5-carboxylate

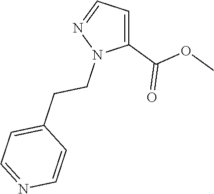 [SMILES: COC(=O)c1ccnn1CCc1ccncc1]

To a solution of 2-(pyridin-4-yl)ethan-1-ol (500 mg, 4.06 mmol, 1 eq) and in dry THE (10 mL) were added methyl 1H-pyrazole-5-carboxylate (500 mg, 4.06 mmol, 1 eq), PPh3 (1.59 g, 6.09 mmol, 1.5 eq) and DIAD (820 mg, 6.09 mmol, 1.5 eq). After stirring at room temperature for 5 h, the reaction mixture was concentrated under reduced pressure. The residue was purified by silica gel column (DCM/MeOH=100/1, v/v) to give a mixture of desired product (930 mg, 4.06 mmol, 99% yield) as a yellow oil.

LCMS (Waters-QDa-02): Rt 1.90 min; [M+1]⁺=231.9

### Step 2: Synthesis of 1-(2-(pyridin-4-yl)ethyl)-1H-pyrazole-5-carboxylic acid

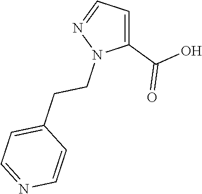 [SMILES: O=C(O)c1ccnn1CCc1ccncc1]

To a solution of methyl 1-(2-(pyridin-4-yl)ethyl)-1H-pyrazole-5-carboxylate (930 mg, 4 mmol, 1 eq) in MeOH (9 mL) and H₂O (3 ml) was added LiOH (193 mg, 2 mmol, 2 eq). After stirring at room temperature for 2 h, the reaction mixture was adjusted PH to 1-2, by HCl aqueous solution (3 M) and concentrated to give the desired product (868 mg, 4 mmol, 100% yield) as a yellow oil.

LCMS (Waters-QDa-02): Rt 1.90 min; [M+1]⁺=217.9

### Step 3: Synthesis of N-(4-(2-isopropoxypropan-2-yl)thiazol-2-yl)-1-(2-(pyridin-4-yl)ethyl)-1H-pyrazole-5-carboxamide

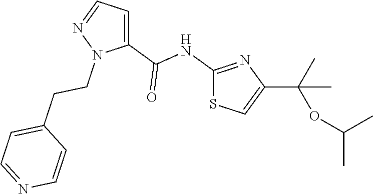 [SMILES: CC(C)OC(C)(C)c1csc(NC(=O)c2ccnn2CCc2ccncc2)n1]

To a mixture of 1-(2-(pyridin-4-yl)ethyl)-1H-pyrazole-5-carboxylic acid (216 mg, 0.99 mmol, 2 eq) in DMA (3 mL) were added 4-(2-isopropoxypropan-2-yl)thiazol-2-amine (100 mg, 0.49 mmol, 1 eq), EDCI (143 mg, 0.74 mmol, 1.5 eq) and DMAP (152 mg, 1.24 mmol, 2.5 eq). After stirring at 100° C. for 3 h in microwave, the reaction mixture was extracted with EtOAc (5 mL×3), washed with water (5 mL×3), brine (5 ml), dried over NaSO3 and concentrated to give a crude, the crude was purification with prep.TLC (DCM/MeOH=10:1, v/v) to give the desired product (29 mg, 0.49 mmol, 14%) as a yellow solid.

LCMS (Agilent-1290): Rt: 2.29 min, m/z [M+1]+=400.2

HNMR (400 MHz, DMSO-d6): δ ppm: 12.65 (s, 1H), 8.41-8.39 (m, 2H), 7.56 (s, 1H), 7.35-7.34 (m, 1H), 7.16-7.11 (m, 3H), 4.85-4.81 (m, 2H), 3.65-3.59 (m, 1H), 3.13-3.10 (m, 2H), 1.50 (s, 6H), 0.93-0.92 (m, 6H).

HPLC (Agilent-1200-A2): Rt: 6.41 min, 94% purity.

### Synthesis of Compound 25

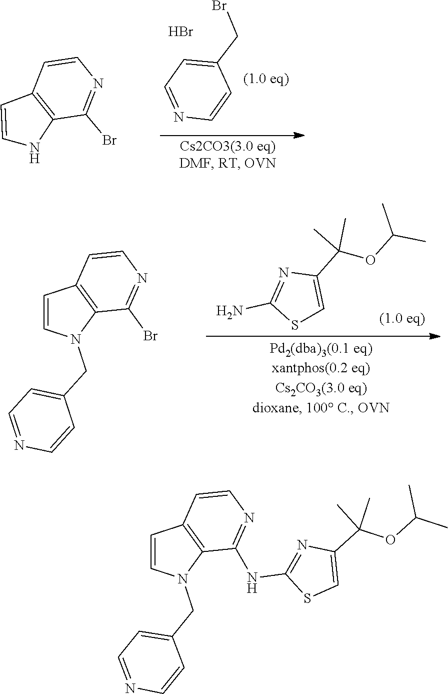 [SMILES: Brc1nccc2cc[nH]c12 ; BrCc1ccncc1 ; Br ; Brc1nccc2ccn(Cc3ccncc3)c12 ; CC(C)OC(C)(C)c1csc(N)n1 ; CC(C)OC(C)(C)c1csc(Nc2nccc3ccn(Cc4ccncc4)c23)n1]

### Step 1: Synthesis of 7-bromo-1-(pyridin-4-ylmethyl)-1H-pyrrolo[2,3-c]pyridine

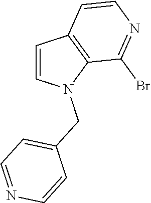 [SMILES: Brc1nccc2ccn(Cc3ccncc3)c12]

To a solution of 7-bromo-1H-pyrrolo[2,3-c]pyridine (100 mg, 0.5 mmol, 1 eq) in DMF (5 mL) were added 4-(bromomethyl)pyridine (129 mg, 0.5 mmol, 1 eq) and Cs₂CO₃ (498 mg, 1.5 mmol, 3.0 eq). After stirring at room temperature for 16 h, the reaction was extracted with EtOAc (5 mL×3), washed with water (5 mL×3), brine (5 ml), dried over NaSO₄ and concentrated to give a crude, the crude was purified by prep.TLC (PE/EtOAc=1/1, v/v) to give the desired product (110 mg, 0.5 mmol, 76%) as a white oil.

LCMS (Waters-QDa-02): Rt 1.80 min; [M+1]⁺=288.1/290.2

### Step 2: Synthesis of 4-(2-isopropoxypropan-2-yl)-N-(1-(pyridin-4-ylmethyl)-1H-pyrrolo[2,3-c]pyridin-7-yl)thiazol-2-amine

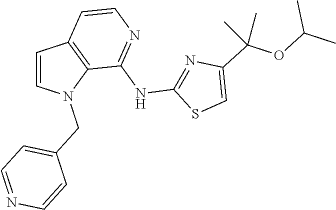 [SMILES: CC(C)OC(C)(C)c1csc(Nc2nccc3ccn(Cc4ccncc4)c23)n1]

To a solution of 7-bromo-1-(pyridin-4-ylmethyl)-1H-pyrrolo[2,3-c]pyridine (110 mg, 0.38 mmol, 1 eq) in dioxane (5 mL) were added 4-(2-isopropoxypropan-2-yl)thiazol-2-amine (76 mg, 0.38 mmol, 1 eq), Pd2(dba)₃ (39 mg, 0.03 mmol, 0.1 eq), xantphos (44 mg, 0.06 mmol, 0.2 eq) and Cs₂CO₃ (374 mg, 1.14 mmol, 3.0 eq). After stirring at 100° C. for 16 h, the reaction mixture was concentrated in vacuo. The residue was purified by prep.TLC (DCM/MeOH=10/1, v/v) to give the desired product (107 mg, 0.38 mmol, 69%) as a white solid.

LCMS (SHIMADZU-2020-P2): Rt: 3.15 min, m/z [M+1]+=408.2

HNMR (400 MHz, DMSO-d6): δ ppm: 13.95 (s, 1H), 8.46 (s, 2H), 7.67 (s, 1H), 7.47 (s, 1H), 7.07 (s, 2H), 6.84 (s, 1.5H), 6.52 (s, 1H), 5.98 (s, 2H), 3.65 (s, 1H), 1.49 (s, 6H), 0.96 (s, 6H).

HPLC (Agilent-1200-A2): Rt: 8.19 min, 95% purity.

### Synthesis of Compound 26

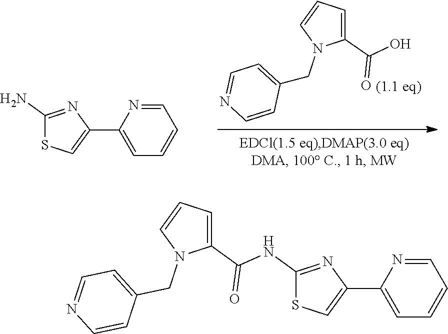 [SMILES: Nc1nc(-c2ccccn2)cs1 ; O=C(O)c1cccn1Cc1ccncc1 ; O=C(Nc1nc(-c2ccccn2)cs1)c1cccn1Cc1ccncc1]

### Step 1: N-(4-(pyridin-2-yl)thiazol-2-yl)-1-(pyridin-4-ylmethyl)-1H-pyrrole-2-carboxamide

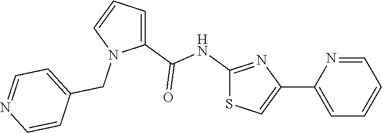 [SMILES: O=C(Nc1nc(-c2ccccn2)cs1)c1cccn1Cc1ccncc1]

To a solution of 4-(pyridin-2-yl)thiazol-2-amine (200 mg, 1.13 mmol, 1 eq) in DMA (3 mL) were added 1-(pyridin-4-ylmethyl)-1H-pyrrole-2-carboxylic acid (295 mg, 1.24 mmol, 1.1 eq), EDCI (324 mg, 1.69 mmol, 1.5 eq) and DMAP (414 mg, 3.39 mmol, 3.0 eq). After stirring at 100° C. for 1 h in microwave, the reaction was extracted with EtOAc (5 mL×3), washed with water (5 mL×3), brine (5 ml), dried over NaSO4 and concentrated to give a crude, the crude was purified by prep.TLC (DCM/MeOH=10/1, v/v) to give the desired product (20 mg, 1.13 mmol, 4.9%) as a white solid.

LCMS (Agilent-1290): Rt: 2.05 min, m/z [M+1]⁺=362.2/363.2

HNMR (400 MHz, DMSO-d6): δ ppm: 12.35 (s, 1H), 8.60-8.58 (m, 1H), 8.49-8.47 (m, 2H), 8.13 (s, 1.5H), 8.00-7.98 (m, 1H), 7.89-7.85 (m, 1H), 7.77 (s, 1H), 7.57-7.55 (m, 1H), 7.35-7.30 (m, 2H), 6.98-6.97 (m, 2H), 6.30-6.29 (m, 1H), 5.70 (s, 2H).

HPLC (Agilent-1200-A2): Rt: 7.78 min, 98% purity.

### Synthesis of Compound 27

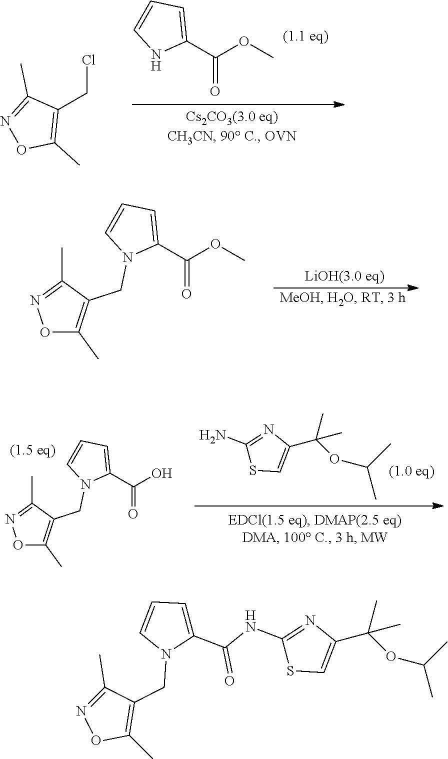

### Synthesis of methyl 1-((3,5-dimethylisoxazol-4-yl)methyl)-1H-pyrrole-2-carboxylate

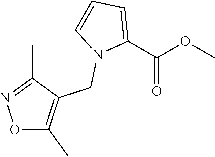 [SMILES: COC(=O)c1cccn1Cc1c(C)noc1C]

To a solution of 4-(chloromethyl)-3,5-dimethylisoxazole (2 g, 0.014 mol, 1 eq) in CH₃CN (30 mL) were added methyl 1H-pyrrole-2-carboxylate (1.9 g, 0.015 mol, 1.1 eq) and Cs₂CO₃ (13.5 g, 0.042 mol, 3 eq) at RT. After stirring at 90° C. for 16 h, the reaction mixture was concentrated under reduced pressure. The residue was purified by silica gel column (PE/EtOAc=2/1, v/v) to give a mixture of desired product (2 g, 0.014 mol, 62%) as a white solid.

LCMS (Waters-QDa-02): Rt 1.89 min; [M+1]⁺=235.0

### Step 2: Synthesis of 1-((3,5-dimethylisoxazol-4-yl)methyl)-1H-pyrrole-2-carboxylic acid

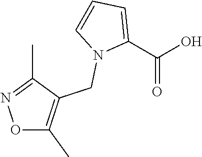 [SMILES: Cc1noc(C)c1Cn1cccc1C(=O)O]

To a solution of methyl 1-((3,5-dimethylisoxazol-4-yl)methyl)-1H-pyrrole-2-carboxylate (1 g, 0.0043 mol, 1 eq) in MeOH (12 mL) and H₂ (4 mL) was added LiOH (305 mg, 0.0129 mmol, 3 eq). After stirring at room temperature for 4 h, the reaction mixture was acid adjustment, and extracted with EtOAc (5 mL×3), washed with water (5 mL×3), brine (5 mL), dried over Na₂SO₄ and concentrated in vacuo give the desired product (400 mg, 0.0043 mol, 44%) as a yellow solid.

LCMS (Water-QDa-02): Rt 1.34 min; [M+1]⁺=221.1

### Step 3: Synthesis of 1-((3,5-dimethylisoxazol-4-yl)methyl)-N-(4-(2-isopropoxypropan-2-yl)thiazol-2-yl)-1H-pyrrole-2-carboxamide

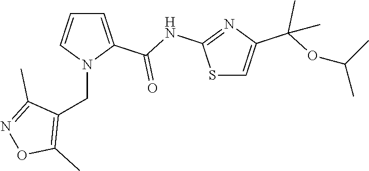 [SMILES: Cc1noc(C)c1Cn1cccc1C(=O)Nc1nc(C(C)(C)OC(C)C)cs1]

To a mixture of 1-((3,5-dimethylisoxazol-4-yl)methyl)-1H-pyrrole-2-carboxylic acid (165 mg, 0.75 mmol, 1.5 eq) in DMA (3 mL) were added 4-(2-isopropoxypropan-2-yl)thiazol-2-amine (100 mg, 0.5 mmol, 1 eq) and EDCI (144 mg, 0.75 mmol, 1.5 eq) and DMAP (153 mg, 1.25 mmol, 2.5 eq). After stirring at 100° C. for 3 h in microwave, the reaction mixture was extracted with EtOAc (5 mL×3), washed with water (5 mL×3), brine (5 mL), dried over Na₂SO₄ and concentrated in vacuo. The residue was purified by prep.TLC (DCM/MeOH=10/1, v/v) to give the desired product (50 mg, 0.75 mmol, 25%) as a white oil.

LCMS (Agilent-1290): Rt: 3.62 min, m/z [M+1]⁺=403.1.

HNMR (400 MHz, DMSO-d6): δ ppm: 12.18 (s, 1H), 7.39-7.38 (m, 1H), 7.20-7.19 (m, 1H), 7.00 (s, 1H), 6.17-6.15 (d, J=8 Hz, 1H), 5.41 (s, 2H), 3.65-3.56 (m, 1H), 2.26 (s, 3H), 1.99 (s, 3H), 1.48 (s, 6H), 0.92-0.91 (d, J=4 Hz, 6H).

HPLC (Agilent-1200-A2): Rt: 13.71 min, 99% purity.

### Synthesis of Compound 28

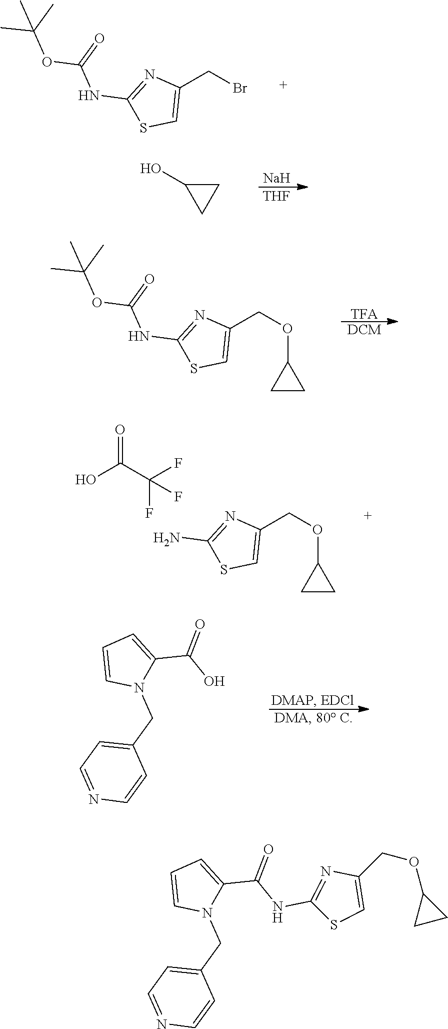

### Step 1

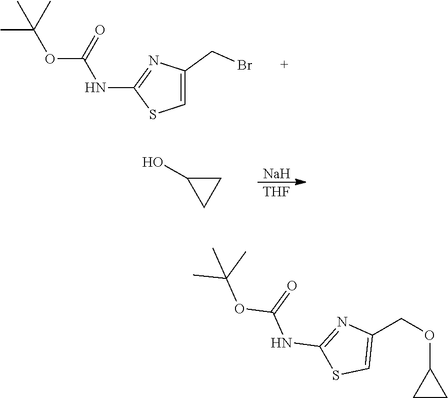

A 20 mL vial with stir bar was charged with cyclopropanol (86.4 μL, 4 eq., 1.36 mmol) in tetrahydrofuran (3 mL, 0.11 M). Sodium hydride 60% w/w (55.9 mg, 4.1 eq., 1.4 mmol) was added to the reaction mixture at 0° C. and stirred at room temperature for 30 minutes. After 30 minutes, tert-butyl N-[4-(bromomethyl)-1,3-thiazol-2-yl]carbamate (0.1 g, 1 eq., 341 μmol) was added to the reaction mixture at 0° C., and stirred at room temperature overnight. The reaction mixture was diluted with saturated aqueous NH₄Cl (5 mL), extracted with EtOAc (5 mL×3), and washed with brine. The organic layers were dried over MgSO₄, filtered, and concentrated in vacuo. The crude material was purified via normal-phase silica gel chromatography (0-100% EtOAc in hexanes) to give the desired product (63 mg, 68% yield). LCMS (+ESI): calc. [M+H]+=271; found 271.

### Step 2

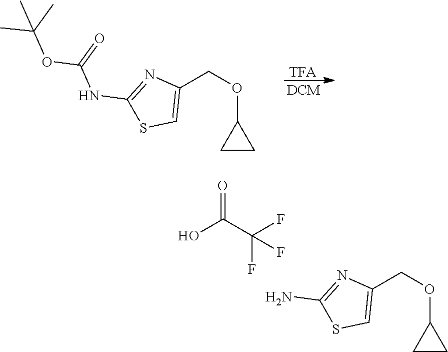

An 8 mL vial with stir bar was charged with tert-butyl N-[4-(cyclopropoxymethyl)-1,3-thiazol-2-yl]carbamate (63 mg, 1 eq., 233 μmol) and DCM (2 mL, 0.12 M). Trifluoroacetic acid (0.5 mL, 28 eq., 6.53 mmol) was added and the reaction mixture stirred at room temperature for 3 hours. The reaction mixture was concentrated in vacuo to give the desired product, assuming quantitative yield (66 mg, 100% yield). LCMS (+ESI): calc. [M+H]+=171; found 171.

### Step 3

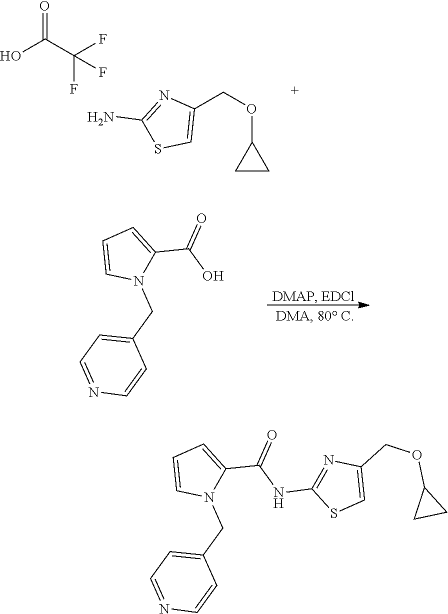

A 4 ml vial with stir bar was charged with 1-[(pyridin-4-yl)methyl]-1H-pyrrole-2-carboxylic acid (39 mg, 1 eq., 193 μmol), 4-(cyclopropoxymethyl)-1,3-thiazol-2-amine; trifluoroacetic acid (66 mg, 1.2 eq., 231 μmol), 4-(dimethylamino)pyridin-1-ium (95 mg, 4 eq., 771 μmol), ({[3-(dimethylamino)propyl]imino}methylidene)(ethyl)amine hydrochloride (55.5 mg, 1.5 eq., 289 μmol), and dimethylacetamide (750 μL, 0.26 M). The reaction mixture was stirred at 80° C. for 3 hours. The crude reaction mixture was purified directly via C₁₈ reverse-phase silica gel chromatography (0-100% ACN in water) to give the desired product (12.5 mg, 18% yield). LCMS (+ESI): calc. [M+H]+=355; found 355.

### Synthesis of Compound 29

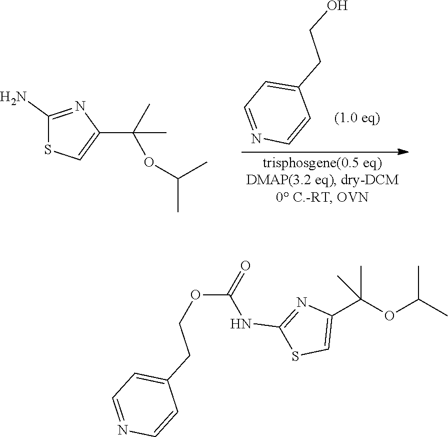 [SMILES: CC(C)OC(C)(C)c1csc(N)n1 ; OCCc1ccncc1 ; CC(C)OC(C)(C)c1csc(NC(=O)OCCc2ccncc2)n1]

### Step 1: 2-(pyridin-4-yl)ethyl (4-(2-isopropoxypropan-2-yl)thiazol-2-yl)carbamate

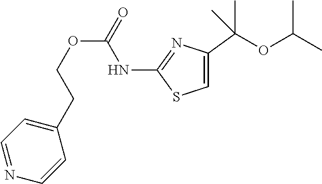 [SMILES: CC(C)OC(C)(C)c1csc(NC(=O)OCCc2ccncc2)n1]

To a solution of 4-(2-isopropoxypropan-2-yl)thiazol-2-amine (100 mg, 0.49 mmol, 1 eq) in DCM (5 mL) were added Triphosgene (74 mg, 0.24 mmol, 0.5 eq), DMAP (195 mg, 1.59 mmol, 3.2 eq) at 0° C. The solution was stirred at 0° C. for 2 h. 2-(pyridin-4-yl)ethan-1-ol (61 mg, 0.49 mmol, 1 eq) was added to the solution. After stirring at room temperature for 16 h. The solution was extracted with DCM (3 mL×3), washed with water (5 mL×3), brine (5 mL), dried over Na₂SO₄ and concentrated to give a crude, the crude was purification by prep.TLC (DCM/MeOH=20:1, v/v) to give the desired product (100 mg, 0.49 mmol, 57%) as a white solid.

LCMS (Agilent-1290): Rt: 2.15 min, m/z [M+1]+=350.1

### HNMR (400 MHz, DMSO-d6): δ ppm: 11.77 (s, 1H), 8.48-8.46 (m, 2H), 7.34-7.32 (m, 2H), 6.98 (s, 1H), 4.39-4.35 (m, 2H), 3.61-3.53 (m, 1H), 2.7-2.94 (m, 2H), 1.43 (s, 6H), 0.90-0.89 (m, 6H).

HPLC (Agilent-1200-A2): Rt: 8.65 min, 96% purity.

### Synthesis of Compounds 48 and 49

### Intermediate 1-2

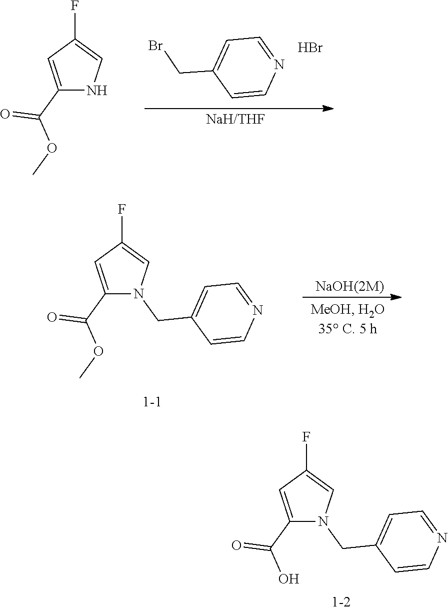

### Step 1: methyl 4-fluoro-1-(pyridin-4-ylmethyl)-1H-pyrrole-2-carboxylate

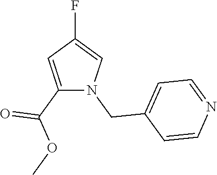 [SMILES: COC(=O)c1cc(F)cn1Cc1ccncc1]

To a solution of methyl 4-fluoro-1H-pyrrole-2-carboxylate (2 g, 14 mmol, 1 eq) in THE (20 mL) was slowly added NaH (60%, 1.7 g, 42 mmol, 3 eq) at 0° C. under N₂. The mixture was stirred at r. t. for 1 h. 4-(bromomethyl)pyridine hydrobromide (7.1 g, 28 mmol, 2 eq) was added and reaction mixture was stirred at RT overnight. The reaction mixture was adjusted to pH=5 with HCl and concentrated under reduced pressure. The residue was purified by silica gel column (DCM/MeOH=20/1, v/v) to give the product (1.6 g, 50%) as a brown solid.

LCMS (Waters-QDa-02): Rt 0.93 min; [M+1]⁺=235.0

### Step 2: 4-fluoro-1-(pyridin-4-ylmethyl)-1H-pyrrole-2-carboxylic acid

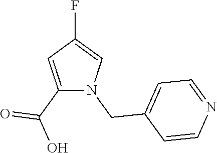 [SMILES: O=C(O)c1cc(F)cn1Cc1ccncc1]

To a solution of methyl 4-fluoro-1-(pyridin-4-ylmethyl)-1H-pyrrole-2-carboxylate (900 mg, 3.84 mmol, 1 eq) in MeOH (4.5 mL) and H₂ (2 mL) was added NaOH (306 mg, 7.68 mmol, 2 eq). After stirring at 35° C. for 4 h, the reaction mixture was acid adjustment, and concentrated in vacuo give the desired product (1400 mg, contain NaCl) as a yellow solid.

LCMS (Water-QDa-02): Rt 0.81 min; [M+1]⁺=221

### Intermediate 2-2

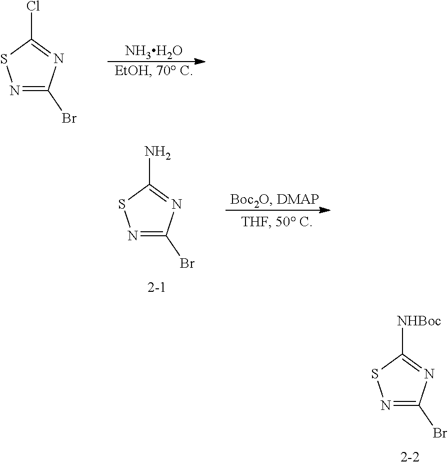 [SMILES: Clc1nc(Br)ns1 ; "NH3·H2O" ; Nc1nc(Br)ns1 ; CC(C)(C)OC(=O)Nc1nc(Br)ns1]

### Step 1: 3-Bromo-1,2,4-thiadiazol-5-amine

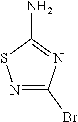

To a solution of 3-bromo-5-chloro-1,2,4-thiadiazole (5 g, 25.1 mmol, 1 eq) in ethanol (15 mL) was added 28% (w/w) ammonia aqueous solution (3.4 mL, 50.1 mmol, 2 eq). The mixture was stirred at 70° C. for 3 h. The reaction mixture was cooled to room temperature, and sodium hydrogen carbonate aqueous solution was added to the mixture. The precipitate was filtered, and the resulting solid was washed with water and dried to afford compound 2-1 (3.8 g, yield:84.4%) as a white solid.

LCMS (Water-QDa-02): Rt 0.89 min; [M+1]⁺=181

### Step 2: tert-butyl (3-bromo-1,2,4-thiadiazol-5-yl)carbamate

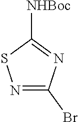 [SMILES: CC(C)(C)OC(=O)Nc1nc(Br)ns1]

To a solution of compound 2-1 (3.8 g 13.5 mmol, 1 eq) in THE (38 mL) were added N,N-dimethyl-4-aminopyridine (82.5 mg, 0.675 mmol, 0.05 eq), di-tert-butyl dicarbonate (3.7 mL, 16.2 mmol, 1.2 eq), and the mixture was stirred at 50° C. for 1 hour. The reaction mixture was evaporated, and to the residue was added dichloromethane-methanol. The insoluble matter in the solution was filtered, the filtrate was evaporated, and the residue was purified by silica-gel column chromatography (PE:EA=10:1) to give compound 2-2 (4.7 g yield:79.6%) as a white solid.

LCMS (Water-QDa-02): Rt 1.25; [M−1]⁺=279

### Compound 49

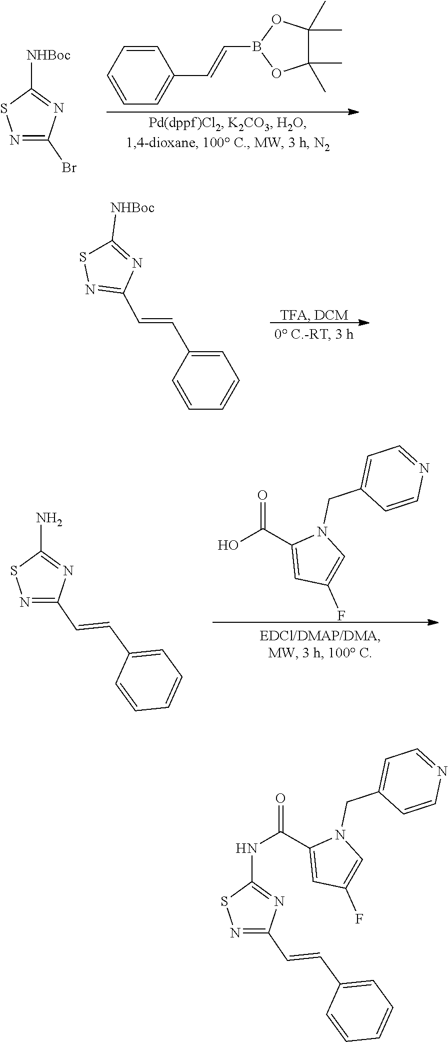 [SMILES: CC(C)(C)OC(=O)Nc1nc(Br)ns1 ; CC1(C)OB(/C=C/c2ccccc2)OC1(C)C ; CC(C)(C)OC(=O)Nc1nc(/C=C/c2ccccc2)ns1 ; Nc1nc(/C=C/c2ccccc2)ns1 ; O=C(O)c1cc(F)cn1Cc1ccncc1 ; O=C(Nc1nc(/C=C/c2ccccc2)ns1)c1cc(F)cn1Cc1ccncc1]

### Step 1: tert-butyl (E)-(3-styryl-1,2,4-thiadiazol-5-yl)carbamate

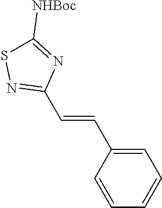 [SMILES: CC(C)(C)OC(=O)Nc1nc(/C=C/c2ccccc2)ns1]

To a solution of tert-butyl (3-bromo-1,2,4-thiadiazol-5-yl)carbamate (200 mg, 0.714 mmol, 1 eq) in H₂O (0.4 mL) and 1,4-dioxane (2 mL) were added (E)-4,4,5,5-tetramethyl-2-styryl-1,3,2-dioxaborolane (492 mg, 2.142 mmol, 3 eq), K₂CO₃ (296 mg, 2.142 mmol, 3 eq), and Pd(dppf)Cl₂ (51 mg, 0.071 mmol, 0.1 eq). The mixture was stirred at 100° C. by microwave under a N₂ atmosphere for 3 h. The reaction mixture was extracted with EtOAc (5 mL×3), washed with water (5 mL×3), brine (5 mL), dried over Na₂SO₄, concentrated in vacuo and purified by revered phase column to give the desired product (145 mg, 0.478 mmol, 67%) as a white solid.

LCMS (Agilent-1290): Rt 2.578 min; [M+1]⁺=304.

### Step 2: (E)-3-styryl-1,2,4-thiadiazol-5-amine

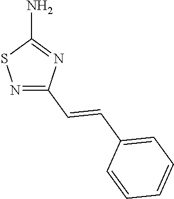 [SMILES: Nc1nc(/C=C/c2ccccc2)ns1]

To a solution of tert-butyl (E)-(3-styryl-1,2,4-thiadiazol-5-yl)carbamate (145 mg, 0.478 mmol, 1 eq) in DCM (1.8 mL) was added TFA (1.8 mL) at 0° C. The mixture was stirred at room temperature for 3 h. The reaction mixture was basified by Na₂CO₃ solution, and extracted with EtOAc (5 mL×3), washed with water (5 mL×3), brine (5 mL), dried over Na₂SO₄, concentrated in vacuo and purified by Prep.TLC (DCM/MeOH=20/1, v/v) to give the desired product (52 mg, 0.256 mmol, 54%) as a white solid.

LCMS (Agilent-1290): Rt 1.514 min; [M+1]⁺=204.

### Step 3: (E)-4-fluoro-1-(pyridin-4-ylmethyl)-N-(3-styryl-1,2,4-thiadiazol-5-yl)-1H-pyrrole-2-carboxamide

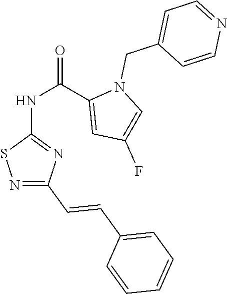 [SMILES: O=C(Nc1nc(/C=C/c2ccccc2)ns1)c1cc(F)cn1Cc1ccncc1]

To a solution of (E)-3-styryl-1,2,4-thiadiazol-5-amine (52 mg, 0.256 mmol, 1 eq) in DMA (1 mL) were added 4-fluoro-1-(pyridin-4-ylmethyl)-1H-pyrrole-2-carboxylic acid (113 mg, 0.512 mmol, 2 eq), EDCI (81 mg, 0.512 mmol, 2 eq) and DMAP (63 mg, 0.512 mmol, 2 eq). The mixture was stirred at 100° C. by microwave for 3 h. The reaction mixture was extracted with EtOAc (5 mL×3), washed with water (5 mL×3), brine (5 mL), dried over Na₂SO₄ and concentrated in vacuo. The residue was purified by prep.TLC (DCM/MeOH=20/1, v/v) to give the desired product (10 mg, 0.025 mmol, 9.6%) as a white solid.

LCMS (Agilent-1290): Rt: 1.750 min, m/z [M+1]⁺=406.

HNMR (400 MHz, DMSO-d6): δ 8.49 (d, J=5.0 Hz, 2H), 7.69-7.58 (m, 3H), 7.41 (t, J=7.4 Hz, 2H), 7.35 (d, J=7.2 Hz, 1H), 7.30 (s, 1H), 7.20 (d, J=11.3 Hz, 2H), 7.03 (d, J=4.9 Hz, 2H), 5.71 (s, 2H).

HPLC (Agilent-1200-A2): Rt: 10.649 min, 96.1% purity.

### Synthesis of Compound 48

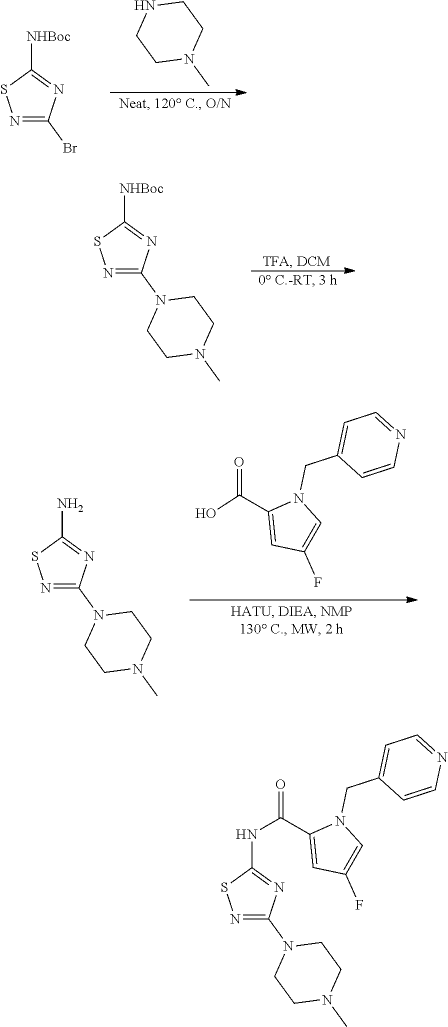

### Step 1: tert-butyl (3-(4-methylpiperazin-1-yl)-1,2,4-thiadiazol-5-yl)carbamate

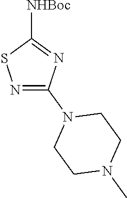 [SMILES: CN1CCN(c2nsc(NC(=O)OC(C)(C)C)n2)CC1]

A solution of tert-butyl (3-bromo-1,2,4-thiadiazol-5-yl)carbamate (500 mg, 1.785 mmol, 1 eq) in 1-methylpiperazine (5 mL) was heated at 120° C. for 16 h. The reaction mixture was extracted with EtOAc (5 mL×3), washed with water (5 mL×3), brine (5 mL), dried over Na₂SO₄, concentrated in vacuo and purified by silica gel column (DCM/MeOH=20/1, v/v) to give the desired product (230 mg, 0.769 mmol, 43%) as a white solid.

LCMS (Agilent-1290): Rt 0.831 min; [M+1]⁺=300.

### Step 2: 3-(4-methylpiperazin-1-yl)-1,2,4-thiadiazol-5-amine

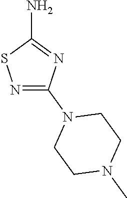

To a solution of tert-butyl (3-(4-methylpiperazin-1-yl)-1,2,4-thiadiazol-5-yl)carbamate (230 mg, 0.769 mmol, 1 eq) in DCM (2 mL) was added TFA (2 mL) at 0° C. The mixture was stirred at room temperature for 3 h, the reaction mixture was basified by Na₂CO₃ solution and extracted with EtOAc (5 mL×3), washed with water (5 mL×3), brine (5 mL), dried over Na₂SO₄, and concentrated in vacuo to give the desired product (90 mg, 0.452 mmol, 59%) as a white solid.

LCMS (Agilent-1290): Rt 1.500 min; [M+1]⁺=200.

### Step 3: 4-fluoro-N-(3-(4-methylpiperazin-1-yl)-1,2,4-thiadiazol-5-yl)-1-(pyridin-4-ylmethyl)-1H-pyrrole-2-carboxamide

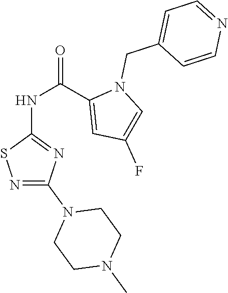 [SMILES: CN1CCN(c2nsc(NC(=O)c3cc(F)cn3Cc3ccncc3)n2)CC1]

To a solution of 3-(4-methylpiperazin-1-yl)-1,2,4-thiadiazol-5-amine (90 mg, 0.452 mmol, 1 eq) in NMP (1 mL) were added 4-fluoro-1-(pyridin-4-ylmethyl)-1H-pyrrole-2-carboxylic acid (198 mg, 0.903 mmol, 2 eq), HATU (343 mg, 0.903 mmol, 2 eq) and DIEA (175 mg, 1.354 mmol, 3 eq). The mixture was stirred at 130° C. by microwave for 2 h. The reaction mixture was extracted with EtOAc (5 mL×3), washed with water (5 mL×3), brine (5 mL), dried over Na₂SO₄ and concentrated in vacuo. The residue was purified by prep.TLC (DCM/MeOH=10/1, v/v) to give the desired product (5 mg, 0.012 mmol, 2.8%) as a yellow solid.

LCMS (Agilent-1290): Rt: 0.922 min, m/z [M+1]⁺=402.

HNMR (400 MHz, DMSO-d6): δ 8.60-8.38 (m, 2H), 7.43-7.32 (m, 2H), 7.00 (d, J=4.9 Hz, 2H), 5.61 (s, 2H), 3.51 (d, J=5.1 Hz, 4H), 2.37 (t, J=5.0 Hz, 4H), 2.20 (s, 3H).

HPLC (Agilent-1200-A3): Rt: 8.290 min, 96.8% purity.

### Synthesis of Compound 37

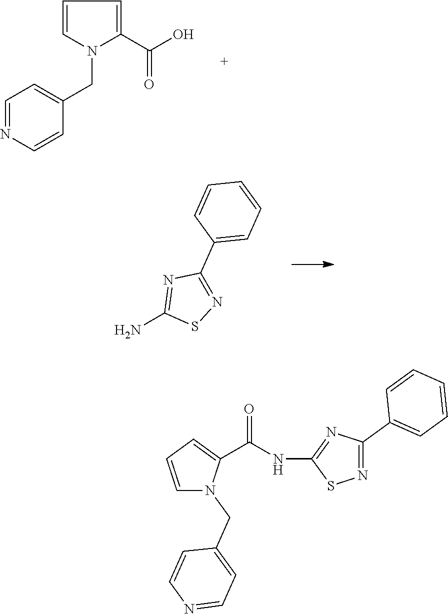 [SMILES: O=C(O)c1cccn1Cc1ccncc1 ; Nc1nc(-c2ccccc2)ns1 ; O=C(Nc1nc(-c2ccccc2)ns1)c1cccn1Cc1ccncc1]

A mixture of 1-[(pyridin-4-yl)methyl]-1H-pyrrole-2-carboxylic acid (50 mg, 247 μmol), 3-tert-butyl-1H-1,2,4-triazol-5-amine (30 mg, 214 μmol) N-(3-tert-butyl-1H-1,2,4-triazol-5-yl)-1-[(pyridin-4-yl)methyl]-1H-pyrrole-2-carboxamide (55 mg, 170 μmol), and EDCI (57.6 mg, 1.5 eq., 371 μmol) in dimethylacetamide (578 μL, 6.21 mmol) was heated at 80° C. for 24 hour. The reaction mixture was diluted with methanol and the product precipitated as a yellow solid, which was collected by filtration. LCMS [M+H]+ expected=362.4. Found=362.0

### Synthesis of Compound 50

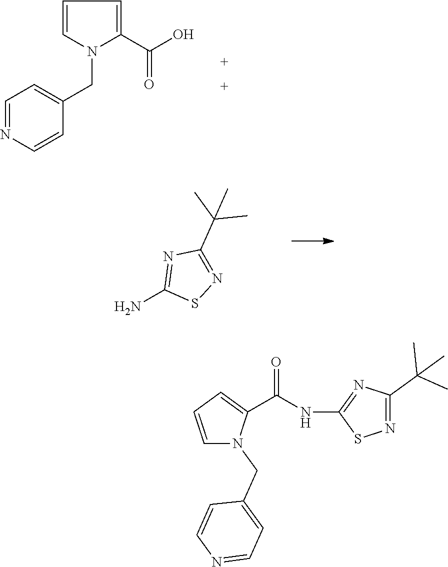

A mixture of 1-[(pyridin-4-yl)methyl]-1H-pyrrole-2-carboxylic acid (50 mg, 247 μmol), 3-tert-butyl-1,2,4-thiadiazol-5-amine (38.9 mg, 247 μmol) N-(3-tert-butyl-1H- and EDCI (57.6 mg, 1.5 eq., 371 μmol) and 4-(dimethylamino)pyridin-1-ium (60.9 mg, 2 eq., 495 μmol) in dimethylacetamide (578 μL, 6.21 mmol) was heated at 80° C. for 24 hour. The reaction mixture was subjected directly to chromatography C₁₈, ISCO, 5-95 ACN/water. LCMS [M+H]+ expected=342.4. Found=342.0

### Example 2. Identifying Molecules that Inhibit PrP Synthesis

Flp-In 293 T-Rex™ cells are transfected with pcDNATM5/FRT/TO plasmid inserted with cDNA encoding Prion protein signal sequence plus 10 amino acids (PrionSS) fused to Gaussia Luciferase (GLuc) (SEQ ID NO: 1). Transfected cells are selected for resistance to the selectable markers Hygromycin and Blasticidin to create a stable cell line that contains the PrionSS-GLuc cDNA insert whose expression is regulated under the T-Rex™ system. The day before assay, cells are trypsinized and plated in 384-well tissue culture plates. The next day, compound dilutions in DMSO/media containing doxycycline are added to the wells and incubated at 37° C., 5% CO2. 24 hours later, coelenterazine substrate is added to each well and luciferase signal is quantified using Tecan Infinite M1000 Pro for potency determination.

Treatment with compound reduces the amount of PrionSS-GLuc protein expressed as measured by reduced luminescence, which indicates that the compound inhibits the secretion of, and thus the biosynthesis of, Prion protein.

The sequence of the PrionSS-Gluc insert is SE ID NO: 1):

MANLGCWMLVLFVATWSDLGLCKKRPKPGGWNKPTENNEDFNIVAVASN
FATTDLDADRGKLPGKKLPLEVLKEMEANARKAGCTRGCLICLSHIKCT
PKMKKFIPGRCHTYEGDKESAQGGIGEAIVDIPEIPGFKDLEPMEQFIA
QVDLCVDCTTGCLKGLANVQCSDLLKKWLPQRCATFASKIQGQVDKIKG
AGGD

### Example 3. HiBit Assay for Quantifying Potency of Protein Secretion Inhibitors

The assay utilized the T-REx™-293 Cell Line, which constitutively expressed the TetR protein, and a plasmid-based transfection, where the signal peptide (SP) for Prion protein (PRP)+10 amino acids form the mature domain (MANLGCWMLVLFVATWSDLGLCKKRPKPGG SEQ ID NO: 2) in-frame with HaloTag-Hibit fusion protein under the control by TetO. Cells were maintained at 37° C. at 5% CO₂ for 24 h after transfection with the plasmid, then treated with compounds. At the same time, doxycycline was added, which enabled expression of the SP-HaloTag-HiBit fusion protein that was secreted from the cells. If the chemical molecules tested were active against the SP of Prion translational translocation of the protein was inhibited and expression of the protein was reduced.

The readout of this assay was the Nano-Glo® HiBiT Extracellular Detection System, a luminescence-based assay where the secreted SP-HaloTag-HiBit fusion protein was quantified by adding a nonlytic detection reagent containing the substrate furimazine and Large BiT (LgBit), the large subunit used for high-affinity binding to HiBit. The HiBit-LgBit-furimazine complex generated a bright, luminescent enzyme. The amount of luminescence generated was proportional to the amount of HiBiT-tagged protein accessible in the medium.

This assay resulted in a dose response curve for each chemical entity, performed as 3-fold dilutions from 30 μM to 1M and includes DMSO (no compound=baseline inhibition) and uninduced (no doxycycline) controls, from which an IC50 was be calculated. The assay, as described here, was designed to test compounds in triplicate against Prion protein.

This assay was modified to test for the inhibition of the secretion of Programmed Cell Death Receptor 1 (PDCD1) and Prion (PRNP). For those experiments, the secretion of SP-HaloTag-HiBit fusion proteins that incorporated the signal peptide+10 amino acids from the mature domain of either PDCD1 or PRNP was measured as described above. The sequence of the SP+10 mature amino acids of PDCD1 was [[MQIPQAPWPVVWAVLQLGWRPGWPLDSPDRPWN (SEQ ID NO:3)]]. The sequence of the SP+10 mature amino acids of PRNP was [[MANLGCWMLVFVATWSDLGLCKKRPKPGGWN (SEQ ID NO: 4)]].

The results of the HiBit assays are summarized in the tables below.

| | HiBit |
|---|---|
| | Assay |
| | IC₅₀ for |
| | PDCD1 |
| | Construct |
| # | (μM) |
| 1 | − |
| 2 | − |
| 3 | − |
| 4 | − |
| 5 | − |
| 6 | − |
| 7 | − |
| 8 | +++ |
| 9 | − |
| 10 | − |
| 11 | − |
| 12 | − |
| 13 | − |
| 14 | − |
| 15 | − |
| 16 | − |
| 17 | − |
| 18 | − |
| 19 | − |
| 20 | − |
| 21 | − |
| 22 | − |
| 23 | − |
| 24 | + |
| 25 | +++ |
| 26 | − |
| 27 | − |
| 28 | − |
| 29 | − |
| 30 | − |
| 31 | − |
| 32 | − |
| 33 | − |
| 35 | − |
| 36 | − |
| 37 | ++++ |
| 38 | + |
| 39 | − |
| 43 | ++++ |
| 44 | + |
| 45 | − |
| 46 | − |
| 47 | − |
| 48 | + |
| 49 | ++++ |
| 50 | ++++ |

++++ stands for <5 μM; +++ stands for 5-10 μM; ++ stands for 10-30 μM; + stands for >30 μM, and − stands for no data.

| | HiBit |
|---|---|
| | Assay |
| | IC₅₀ for |
| | PRP |
| | Construct |
| # | (μM) |
| 1 | + |
| 2 | + |
| 3 | + |
| 4 | ++ |
| 5 | +++ |
| 6 | + |
| 7 | ++ |
| 8 | ++++ |
| 9 | − |
| 10 | ++ |
| 11 | + |
| 12 | + |
| 13 | + |
| 14 | + |
| 15 | + |
| 16 | − |
| 17 | + |
| 18 | + |
| 19 | + |
| 20 | + |
| 21 | + |
| 22 | + |
| 23 | + |
| 24 | + |
| 25 | ++ |
| 26 | − |
| 27 | − |
| 28 | − |
| 29 | − |
| 30 | − |
| 31 | − |
| 32 | − |
| 33 | − |
| 35 | − |
| 36 | − |
| 37 | +++ |
| 38 | + |
| 39 | ++ |
| 43 | +++ |
| 44 | + |
| 45 | + |
| 46 | + |
| 47 | + |
| 48 | + |
| 49 | ++++ |
| 50 | ++++ |

++++ stands for <5 μM; +++ stands for 5-10 μM; ++ stands for 10-30 μM; + stands for >30 μM, and − stands for no data.

### Numbered Embodiments

1. A compound, or pharmaceutically acceptable salt thereof, having the structure of Formula 1:

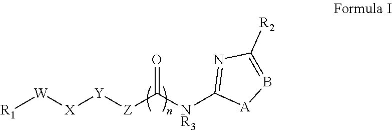

wherein
R₁ is H, optionally substituted C₁-C₆ alkyl, optionally substituted C₃-C₇ cycloalkyl, optionally substituted C₂-C₉ heterocycle, optionally substituted C₆-C₁₀ aryl, optionally substituted C₂-C₉ heteroaryl, C₂-C₉ heteroaryl C₁-C₆ alkyl, or Rₐ-R_{b}-R_{c},
   wherein Rₐ is optionally substituted C₆-C₁₀ arylene or optionally substituted C₂-C₉ heteroarylene;
   R_{b} is —O— or optionally substituted C₁-C₆ alkylene, and
   R_{c} is optionally substituted C₆-C₁₀ aryl;
Z is absent, optionally substituted C₃-C₁₀ cycloalkylene, optionally substituted C₂-C₉ heterocyclylene, C₆-C₁₀ arylene, or optionally substituted C₂-C₉ heteroarylene;
Y is absent, O, or NR₃;
X is absent or optionally substituted C₁-C₆ alkylene;
W is absent, S, O or NR₃;
n is 0 or 1;
A is S, O, NH, or CH;
B is C or N;
R₂ is optionally substituted C₁-C₆ alkyl, optionally substituted C₂-C₆ alkenyl, optionally substituted C₃-C₇ cycloalkyl, optionally substituted C₂-C₉ heterocycle, optionally substituted C₆-C₁₀ aryl, optionally substituted C₂-C₉ heteroaryl; optionally substituted C₂-C₉ heteroaryl C₁-C₆ alkyl, optionally substituted C₃-C₇ cycloalkyl C₁-C₆ aryl, optionally substituted C₁-C₆ heteroalkyl, cyano, or has the structure X₁—O—Y₁;
   wherein X₁ is optionally substituted C₁-C₆ alkylene, and
   Y₁ is optionally substituted C₁-C₆ alkyl, optionally substituted C₃-C₈ cycloalkyl, or optionally
substituted C₆-C₁₀ aryl; and
and
each R₃ is, independently, H or C₁-C₆ alkyl.

2. The compound, or pharmaceutically acceptable salt thereof, of embodiment 1:
wherein R₁ is H, optionally substituted C₁-C₆ alkyl, optionally substituted C₃-C₇ cycloalkyl, optionally substituted C₂-C₉ heterocycle, optionally substituted C₆-C₁₀ aryl, or optionally substituted C₂-C₉ heteroaryl;
Z is absent, optionally substituted C₃-C₁₀ cycloalkyl, optionally substituted C₂-C₉ heterocyclyl, C₆-C₁₀ aryl, or optionally substituted C₂-C₉ heteroaryl;
Y is absent, O, or NR₃;
X is absent or optionally substituted C₁-C₆ alkylene;
W is absent, S, O or NR₃
n is 0 or 1;
A is S, O, or CH;
B is C or N;
R₂ is optionally substituted C₁-C₆ alkyl, optionally substituted C₃-C₇ cycloalkyl, optionally substituted C₂-C₉ heterocycle, optionally substituted C₆-C₁₀ aryl, optionally substituted C₂-C₉ heteroaryl; optionally substituted C₂-C₉ heteroaryl C₁-C₆ alkyl, optionally substituted C₃-C₇ cycloalkyl C₁-C₆ aryl, optionally substituted C₆-C₁₀; and
each R₃ is, independently, H or C₁-C₆ alkyl.

3. The compound, or pharmaceutically acceptable salt thereof, of embodiment 1 or 2, wherein n is 1.

4. The compound, or pharmaceutically acceptable salt thereof, of any one of embodiments 1 to 3, wherein the compound has the structure of Formula II:

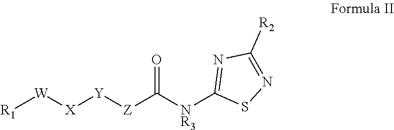

5. The compound, or pharmaceutically acceptable salt thereof, of any one of embodiments 1 to 3, wherein the compound has the structure of Formula III:

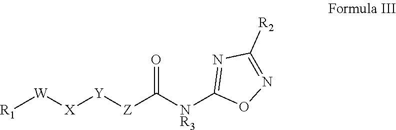

6. The compound, or pharmaceutically acceptable salt thereof, of embodiment 1 or 3, wherein the compound has the structure of Formula IV:

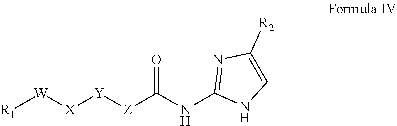

7. The compound, or pharmaceutically acceptable salt thereof, of any one of embodiments 4 to 6, wherein Z is optionally substituted C₆-C₁₀ aryl, or optionally substituted C₂-C₉ heteroaryl.

8. The compound, or pharmaceutically acceptable salt thereof, of any one of embodiments 1 to 7, wherein Y is O or NR₃.

9. The compound, or pharmaceutically acceptable salt thereof, of embodiment 8, wherein Y is O.

10. The compound, or pharmaceutically acceptable salt thereof, of embodiment 8, wherein Y is NR₃.

11. The compound, or pharmaceutically acceptable salt thereof, of embodiment 8, wherein Y is NH.

12. The compound, or pharmaceutically acceptable salt thereof, of any one of embodiments 1 to 11, X is optionally substituted C₁-C₆ alkylene.

13. The compound, or pharmaceutically acceptable salt thereof, of any one of embodiments 1 to 12, wherein W is S, O or NR₃.

14. The compound, or pharmaceutically acceptable salt thereof, of embodiment 13, wherein W is S.

15. The compound, or pharmaceutically acceptable salt thereof, of embodiment 13, wherein W is O.

16. The compound, or pharmaceutically acceptable salt thereof, of embodiment 13, wherein W is NR₃.

17. The compound, or pharmaceutically acceptable salt thereof, of embodiment 16, wherein W is NH.

18. The compound, or pharmaceutically acceptable salt thereof, of any one of embodiments 1 to 3, wherein A is S and B is C.

19. The compound, or pharmaceutically acceptable salt thereof, of any one of embodiments 1 to 3 or 18, wherein the compound has the structure:

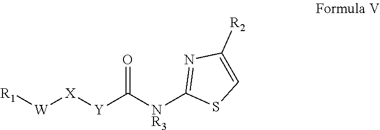

wherein Y is O or NR₃.

20. The compound, or pharmaceutically acceptable salt thereof, of embodiment 19, wherein X is optionally substituted C₁-C₆ alkylene.

21. The compound, or pharmaceutically acceptable salt thereof, of embodiments 19 or 20, wherein Y is 0.

22. The compound, or pharmaceutically acceptable salt thereof, of embodiment 19 or 20, wherein Y is NR₃.

23. The compound, or pharmaceutically acceptable salt thereof, of embodiment 22, wherein Y is NH.

24. The compound, or pharmaceutically acceptable salt thereof, of any one of embodiments 1 to 3 or 18, wherein the compound has the structure of Formula VI:

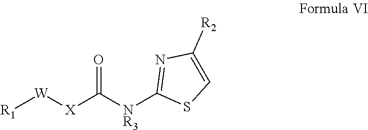

wherein W is NR₃.

25. The compound, or pharmaceutically acceptable salt thereof, of embodiment 24, wherein X is optionally substituted C₁-C₆ alkylene.

26. The compound, or pharmaceutically acceptable salt thereof, of embodiment 24 or 25, wherein W is NH.

27. The compound, or pharmaceutically acceptable salt thereof, of embodiment 1 or 2, wherein n is 0.

28. The compound, or pharmaceutically acceptable salt thereof, of embodiment 27, wherein A is S and B is C.

29. The compound, or pharmaceutically acceptable salt thereof, of any one of embodiments 1 to 6, 8 to 18, 27, or 28, wherein Z is optionally substituted phenylene, optionally substituted pyridinylene, optionally substituted pyrimidinylene, optionally substituted pyridazinylene, optionally substituted pyrazinylene, optionally substituted triazinylene, optionally substituted tetrazinylene, optionally substituted thiophenylene, optionally substituted pyrrolylene, optionally substituted furanylene, optionally substituted pyrazolylene, optionally substituted thiazolylene, optionally substituted oxadiazolylene, optionally substituted thiadiazolylene, optionally substituted isoxazolylene, optionally substituted isothiazolylene, optionally substituted thiazolylene, optionally substituted oxazolylene, optionally substituted imidazolylene, optionally substituted cyclohexylene, optionally substituted cyclopentylene, optionally substituted cyclobutylene, optionally substituted cyclopropylene, optionally substituted cycloheptylene, optionally substituted cyclooctylene, optionally substituted indolylene, or optionally substituted azaindolylene.

30. The compound, or pharmaceutically acceptable salt thereof, of any one of embodiments 1 to 18, or 27 to 29, wherein Z has the structure of Formula VIIi:

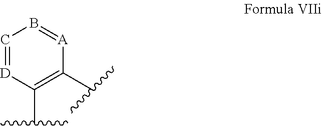

wherein A, B, C and D are, each, independently, N, CH, or CR₄; and
each R₄ is independently optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol.

31. The compound, or pharmaceutically acceptable salt thereof, of embodiment 30, wherein Z has the structure of Formula VIIIi:

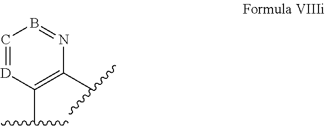

wherein B, C, and D are each, independently, CH or CR₄.

32. The compound, or pharmaceutically acceptable salt thereof, of embodiment 30, wherein Z has the structure of Formula IXi:

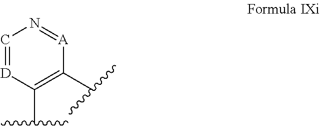

wherein A, C, and D are each, independently, CH or CR₄.

33. The compound, or pharmaceutically acceptable salt thereof, of embodiment 30, wherein Z has the structure of Formula Xi:

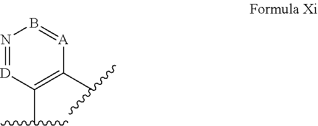

wherein A, B, and D are each, independently, CH or CR₄.

34. The compound, or pharmaceutically acceptable salt thereof, of embodiment 30, wherein Z has the structure of Formula Xli:

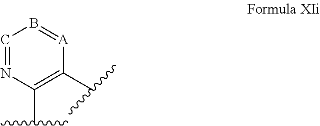

wherein A, B, and C are each, independently, CH or CR₄.

35. The compound, or pharmaceutically acceptable salt thereof, of embodiment 30, wherein Z has the structure of Formula XIIi:

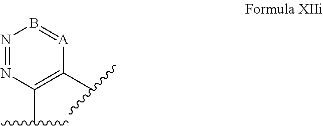

wherein A and B are each, independently, CH or CR₄.

36. The compound, or pharmaceutically acceptable salt thereof, of embodiment 30, wherein Z has the structure of Formula XIIIi:

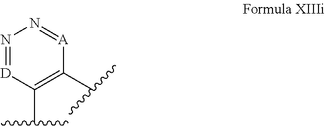

wherein A and D are each, independently, CH or CR₄.

37. The compound, or pharmaceutically acceptable salt thereof, of embodiment 30, wherein Z has the structure of Formula XIVi:

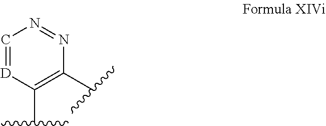

wherein C and D are each, independently, CH or CR₄.

38. The compound, or pharmaceutically acceptable salt thereof, of embodiment 30, wherein Z has the structure of Formula XVi:

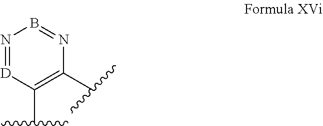

wherein B and D are each, independently, CH or CR₄.

39. The compound, or pharmaceutically acceptable salt thereof, of embodiment 30, wherein Z has the structure of Formula XVIi:

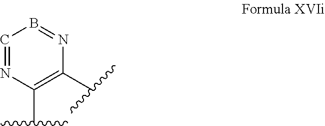

wherein B and C are each, independently, CH or CR₄.

40. The compound, or pharmaceutically acceptable salt thereof, of embodiment 30, wherein Z has the structure of Formula XVIIi:

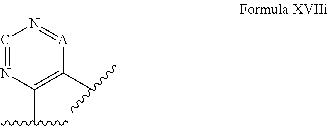

wherein A and C are each, independently, CH or CR₄.

41. The compound, or pharmaceutically acceptable salt thereof, of embodiment 30, wherein Z has the structure of Formula XVIIIi:

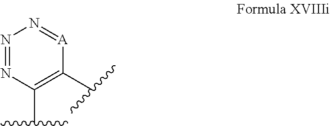

wherein A is CH or CR₄.

42. The compound, or pharmaceutically acceptable salt thereof, of embodiment 30, wherein Z has the structure of Formula XIXi:

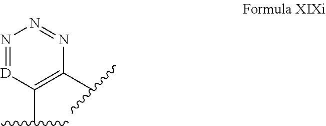

wherein D Is CH or CR₄

43. The compound, or pharmaceutically acceptable salt thereof, of embodiment 30, wherein Z has the structure of Formula XXi:

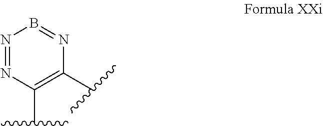

wherein B is CH or CR₄.

44. The compound, or pharmaceutically acceptable salt thereof, of embodiment 30, wherein Z has the structure of Formula XXIi:

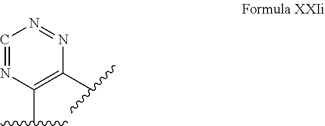

wherein C is CH or CR₄.

45. The compound, or pharmaceutically acceptable salt thereof, of embodiment 30, wherein Z has the structure of Formula XXIIi:

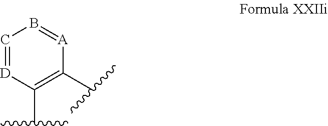

wherein A, B, C, and D are each, independently, CH or CR₄

46. The compound, or pharmaceutically acceptable salt thereof, of any one of embodiments 1 to 6, 8 to 18, or 27 to 29, wherein Z has the structure of Formula XXIlli:

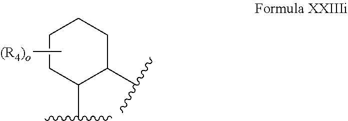

wherein o is 0, 1, 2, 3, or 4; and

each R₄ is, independently, optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol.

47. The compound, or pharmaceutically acceptable salt thereof, of any one of embodiments 1 to 6, 8 to 18, or 27 to 29, wherein Z has the structure of Formula XXIi:

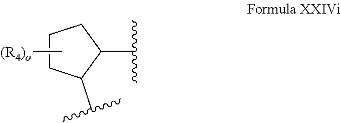

wherein o is 0, 1, 2, or 3; and each R₄ is, independently, optionally substituted aryl, optionally substituted carbocyclyl, halogen, hydroxyl, optionally substituted heteroalkyl, optionally substituted heteroaryl

48. The compound, or pharmaceutically acceptable salt thereof, of any one of embodiments 1 to 18, or 27 to 29, wherein Z has the structure of Formula XXVi:

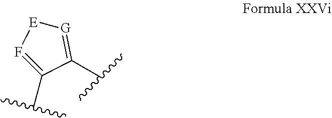

wherein
F and G are each, independently, N, CH, or CR₄;
each R₄ is independently optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol; and E is NH, O, or S.
49. The compound, or pharmaceutically acceptable salt thereof, of embodiment 48, wherein Z has the structure of Formula XXVIi:

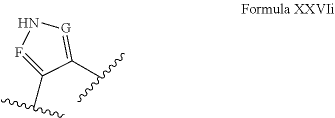

wherein F and G are each, independently, CH or CR₄.

50. The compound, or pharmaceutically acceptable salt thereof, of embodiment 48, wherein Z has the structure of Formula XXVIIi:

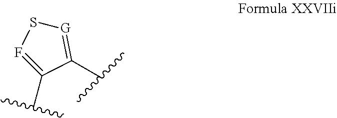

wherein F and G are each, independently, CH or CR₄.

51. The compound, or pharmaceutically acceptable salt thereof, of embodiment 48, wherein Z has the structure of Formula XXVIIIi:

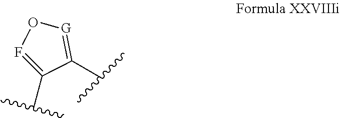

wherein F and G are each, independently, CH or CR₄.

52. The compound, or pharmaceutically acceptable salt thereof, of any one of embodiments 1 to 18, or 27 to 29, wherein Z has the structure of Formula XXIXi:

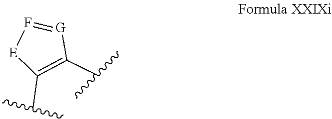

wherein
F and G are each, independently, N, CH, or CR₄;
each R₄ is independently optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol; and
E is NH, O, or S.

53. The compound, or pharmaceutically acceptable salt thereof, of embodiment 52, wherein Z has the structure of Formula XXXi:

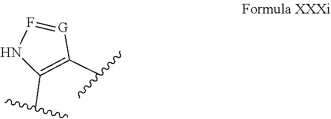

wherein F and G are each, independently, CH or CR₄.

54. The compound, or pharmaceutically acceptable salt thereof, of embodiment 52, wherein Z has the structure of Formula XXXIi:

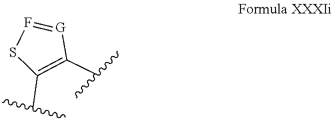

wherein F and G are each, independently, CH or CR₄.

55. The compound, or pharmaceutically acceptable salt thereof, of embodiment 52, wherein Z has the structure of Formula XXXIIi:

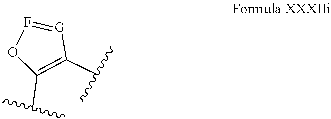

wherein F and G are each, independently, CH or CR₄.

56. The compound, or pharmaceutically acceptable salt thereof, of any one of embodiments 1 to 18, or 27 to 29, wherein Z has the structure of Formula XXXIIIi:

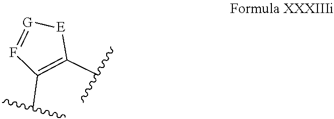

wherein
F and G are each, independently, N, CH, or CR₄;
each R₄ is independently optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol; and
E is NH, O, or S.

57. The compound, or pharmaceutically acceptable salt thereof, of embodiment 56, wherein Z has the structure of Formula XXXIVi:

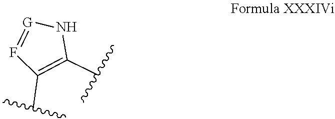

wherein F and G are each, independently, CH or CR₄.

58. The compound, or pharmaceutically acceptable salt thereof, of embodiment 56, wherein Z has the structure of Formula XXXVi:

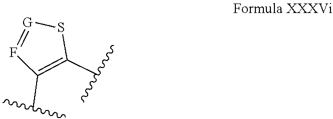

wherein F and G are each, independently, CH or CR₄.

59. The compound, or pharmaceutically acceptable salt thereof, of embodiment 56, wherein Z has the structure of Formula XXXVIi:

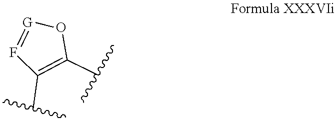

wherein F and G are each, independently, CH or CR₄.

60. The compound, or pharmaceutically acceptable salt thereof, of any one of embodiments 1 to 18, or 27 to 29, wherein Z has the structure of Formula XXXVIIi:

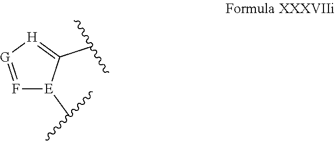

wherein
F, G, and H are each, independently, N, CH, or CR₄;
each R₄ is independently optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol; and
E is N or CH.

61. The compound, or pharmaceutically acceptable salt thereof, of embodiment 60, wherein Z has the structure of Formula XXXVIIIi:

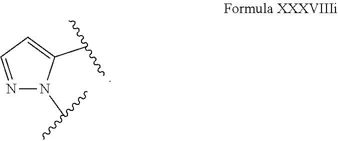 [SMILES: *c1ccnn1*]

62. The compound, or pharmaceutically acceptable salt thereof, of embodiment 60, wherein Z has the structure of Formula XXXIXi:

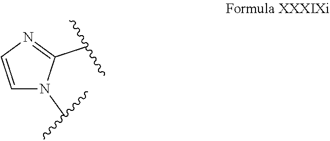 [SMILES: *c1nccn1*]

63. The compound, or pharmaceutically acceptable salt thereof, of embodiment 60, wherein Z has the structure of Formula XXXXi:

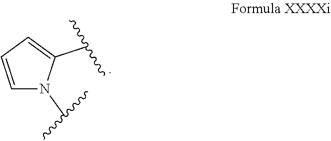 [SMILES: *c1cccn1*]

64. The compound, or pharmaceutically acceptable salt thereof, of embodiment 60, wherein Z has the structure of Formula XXXXIi:

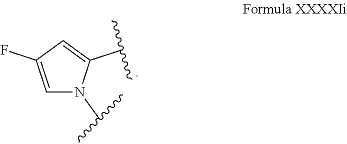 [SMILES: *c1cc(F)cn1*]

65. The compound, or pharmaceutically acceptable salt thereof, of embodiment 60, wherein Z has the structure of Formula XXXXIVi:

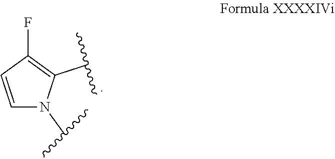 [SMILES: *c1c(F)ccn1*]

66. The compound, or pharmaceutically acceptable salt thereof, of any one of embodiments 1 to 18, or 27 to 29, wherein Z has the structure of Formula XXXXIIi:

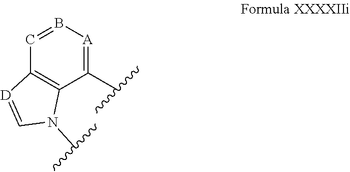

wherein A, B, C, and D are each, independently, CH, CR₄, or N; and
each R₄ is independently optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol.

67. The compound, or pharmaceutically acceptable salt thereof, of embodiment 66, wherein Z has the structure of Formula XXXXIIIi:

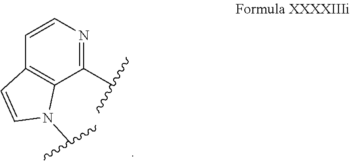 [SMILES: *c1nccc2ccn(*)c12]

68. The compound, or pharmaceutically acceptable salt thereof, of any one of embodiments 1 to 18, or 27 to 29, wherein Z has the structure of Formula XXXXVi:

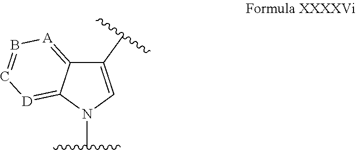

wherein A, B, C, and D are each, independently, CH, CR₄, or N; and
each R₄ is independently optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol

69. The compound, or pharmaceutically acceptable salt thereof, of any one of embodiments 1, 2, or 27 to 29, wherein the compound has the structure of Formula VII:

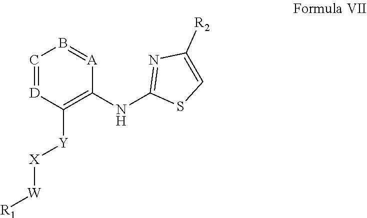

wherein A, B, C and D are, each, independently, N, CH, or CR₄; and
each R₄ is independently optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol.

70. The compound, or pharmaceutically acceptable salt thereof, of embodiment 69, wherein the compound has the structure of Formula VIII:

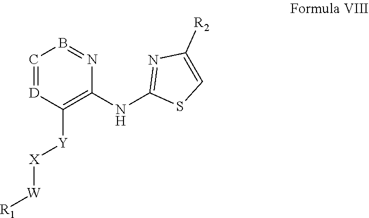

wherein B, C, and D are each, independently, CH or CR₄.

71. The compound, or pharmaceutically acceptable salt thereof, of embodiment 69, wherein the compound has the structure of formula IX:

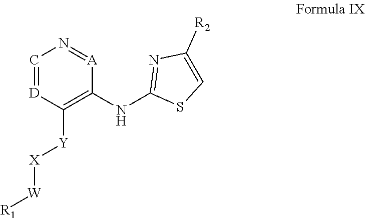

wherein A, C, and D are each, independently, CH or CR₄.

72. The compound, or pharmaceutically acceptable salt thereof, of embodiment 69, wherein the compound has the structure of formula X:

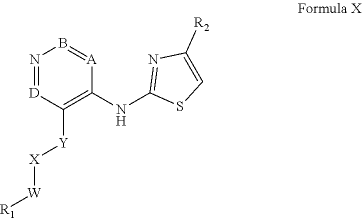

wherein A, B, and D are each, independently, CH or CR₄.

73. The compound, or pharmaceutically acceptable salt thereof, of embodiment 69, wherein the compound has the structure of Formula XI:

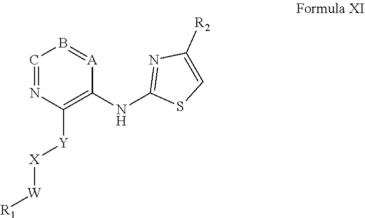

wherein A, B, and C are each, independently, CH or CR₄.

74. The compound, or pharmaceutically acceptable salt thereof, of embodiment 69, wherein the compound has the structure of Formula XII:

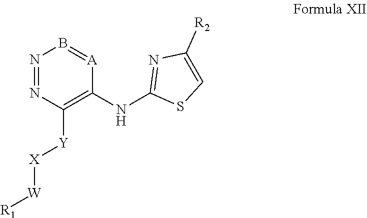

wherein A and B are each, independently, CH or CR₄.

75. The compound, or pharmaceutically acceptable salt thereof, of embodiment 69, wherein the compound has the structure of Formula XIII:

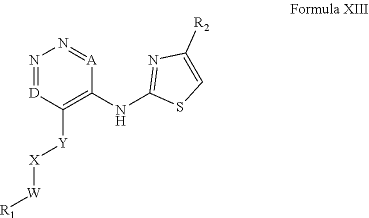

wherein A and D are each, independently, CH or CR₄.

76. The compound, or pharmaceutically acceptable salt thereof, of embodiment 69, wherein the compound has the structure of Formula XIII:

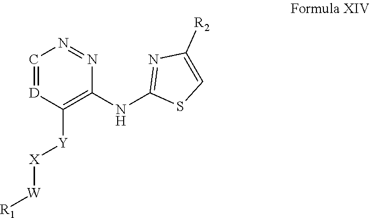

wherein C and D are each, independently, CH or CR₄.

77. The compound, or pharmaceutically acceptable salt thereof, of embodiment 69, wherein the compound has the structure of Formula XV:

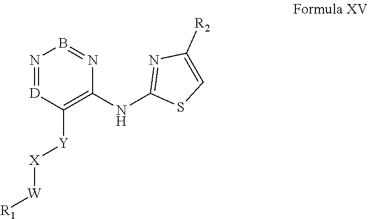

wherein B and D are each, independently, CH or CR₄.

78. The compound, or pharmaceutically acceptable salt thereof, of embodiment 69, wherein the compound has the structure of Formula XVI:

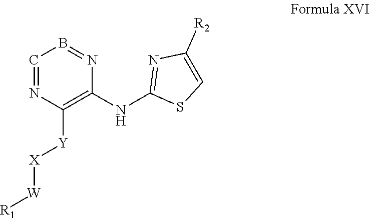

wherein B and C are each, independently, CH or CR₄.

79. The compound, or pharmaceutically acceptable salt thereof, of embodiment 69, wherein the compound has the structure of Formula XVII:

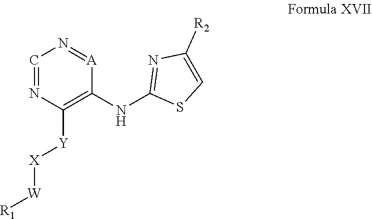

wherein A and C are each, independently, CH or CR₄.

80. The compound, or pharmaceutically acceptable salt thereof, of embodiment 69, wherein the compound has the structure of Formula XVIII:

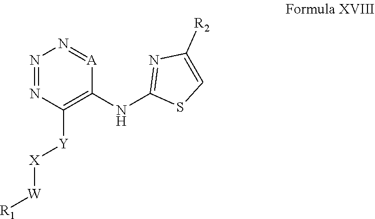

wherein A is CH or CR₄.

81. The compound, or pharmaceutically acceptable salt thereof, of embodiment 69, wherein the compound has the structure of Formula XIX:

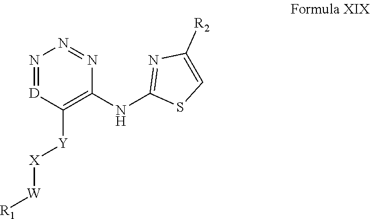

wherein D Is CH or CR₄

82. The compound, or pharmaceutically acceptable salt thereof, of embodiment 69, wherein the compound has the structure of Formula XX:

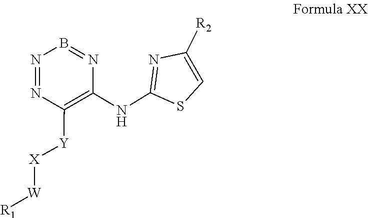

wherein B is CH or CR₄.

83. The compound, or pharmaceutically acceptable salt thereof, of embodiment 69, wherein the compound has the structure of Formula XXI:

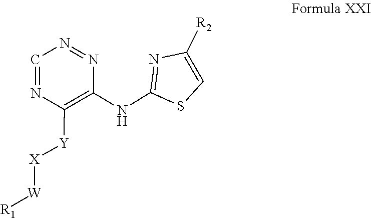

wherein C is CH or CR₄.

84. The compound, or pharmaceutically acceptable salt thereof, of embodiment 69, wherein the compound has the structure of Formula XXII:

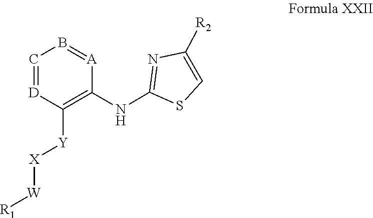

wherein A, B, C, and D are each, independently, CH or CR₄.

85. The compound, or pharmaceutically acceptable salt thereof, of one of embodiments 1, 2, 27 26 to 29, wherein the compound has the structure of Formula XXIII

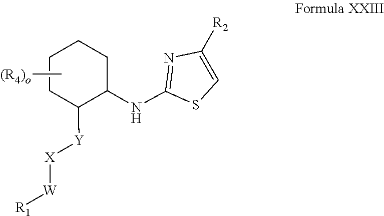

wherein o is 0, 1, 2, 3, or 4; and
each R₄ is, independently, optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol.

86. The compound, or pharmaceutically acceptable salt thereof, of one of embodiments 1, 2, or 27 to 29, wherein the compound has the structure of Formula XXIV:

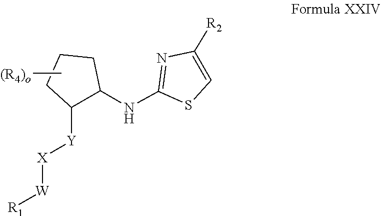

wherein o is 0, 1, 2, or 3; and
each R₄ is, independently, optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol.

87. The compound, or pharmaceutically acceptable salt thereof, of one of embodiments 1, 2, or 27 to 29, wherein the compound has the structure of Formula XXV:

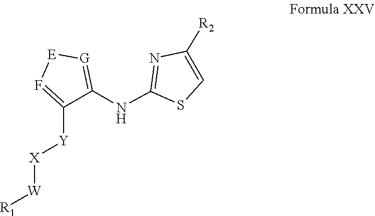

wherein;
each R₄ is independently optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol;
E is NH, O, or S; and
F and G are each, independently, N, CH, or CR₄.

88. The compound, or pharmaceutically acceptable salt thereof, of embodiment 87, wherein the compound has the structure of Formula XXVI:

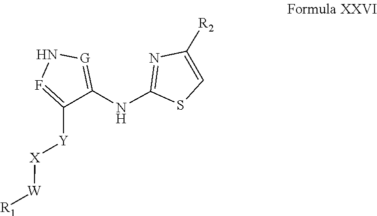

wherein F and G are each, independently, CH or CR₄.

89. The compound, or pharmaceutically acceptable salt thereof, of embodiment 87, wherein the compound has the structure of Formula XXVII:

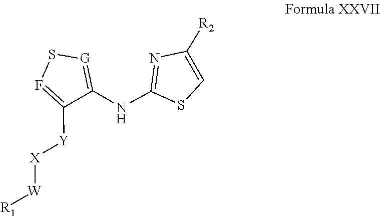

wherein F and G are each, independently, CH or CR₄.

90. The compound, or pharmaceutically acceptable salt thereof, of embodiment 87, wherein the compound has the structure of Formula XXVIII:

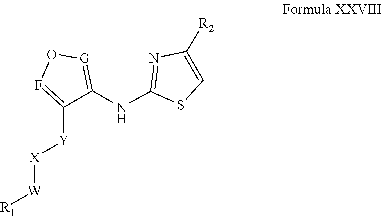

wherein F and G are each, independently, CH or CR₄.

91. The compound, or pharmaceutically acceptable salt thereof, of any one of embodiments 1, 2, or 27 to 29, wherein the compound has the structure of Formula XXIX:

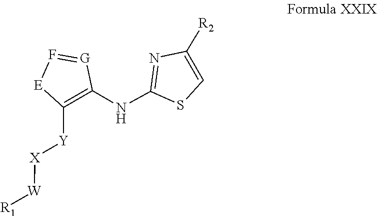

wherein
each R₄ is independently optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol;
E is NH, O, or S; and
F and G are each, independently, N, CH, or CR₄.

92. The compound, or pharmaceutically acceptable salt thereof, of embodiment 91, wherein the compound has the structure of Formula XXX:

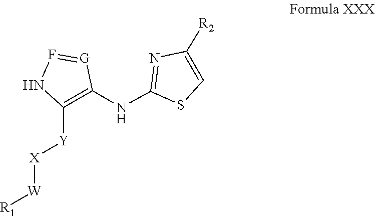

wherein F and G are each, independently, CH or CR₄.

93. The compound, or pharmaceutically acceptable salt thereof, of embodiment 91, wherein the compound has the structure of Formula XXXI:

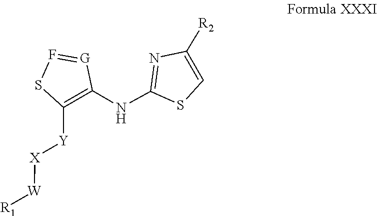

wherein F and G are each, independently, CH or CR₄.

94. The compound, or pharmaceutically acceptable salt thereof, of embodiment 91, wherein the compound has the structure of Formula XXXII:

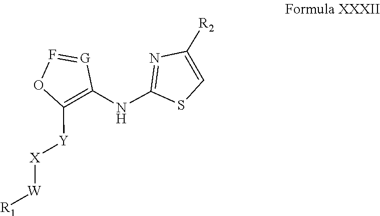

wherein F and G are each, independently, CH or CR₄.

95. The compound, or pharmaceutically acceptable salt thereof, of any one of embodiments 1, 2, or 27 to 29, wherein the compound has the structure of Formula XXXIII:

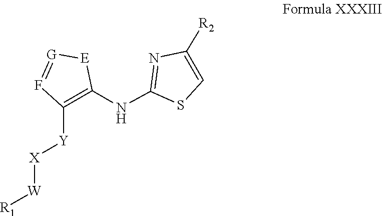

wherein
F and G are each, independently, N, CH, or CR₄;
each R₄ is independently optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol; and
E is NH, O, or S.

96. The compound, or pharmaceutically acceptable salt thereof, of embodiment 95, wherein the compound has the structure of Formula XXXIV:

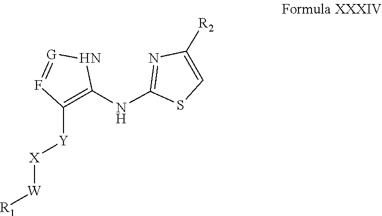

wherein F and G are each, independently, CH or CR₄.

97. The compound, or pharmaceutically acceptable salt thereof, of embodiment 95, wherein the compound has the structure of Formula XXXV:

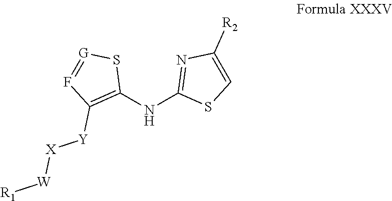

wherein F and G are each, independently, CH or CR₄.

98. The compound, or pharmaceutically acceptable salt thereof, of embodiment 95, wherein the compound has the structure of Formula XXXVI:

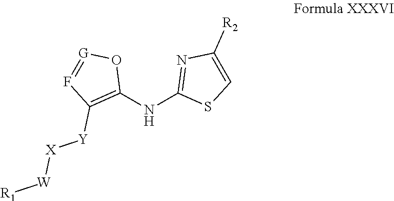

wherein F and G are each, independently, CH or CR₄.

99. The compound, or pharmaceutically acceptable salt thereof, of any one of embodiments 1, 2, or 27 to 29, wherein the compound has the structure of Formula XXXVII:

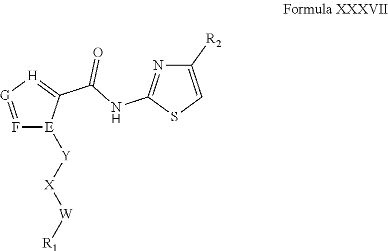

wherein
each R₄ is independently optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol;
E is N or CH
F, G, and H are each, independently, N, CH, or CR₄.

100. The compound, or pharmaceutically acceptable salt thereof, of embodiment 99, wherein the compound has the structure of Formula XXXVIII:

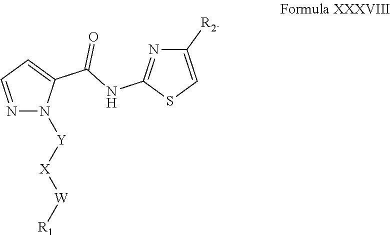

101. The compound, or pharmaceutically acceptable salt thereof, of embodiment 99, wherein the compound has the structure of Formula XXXIX:

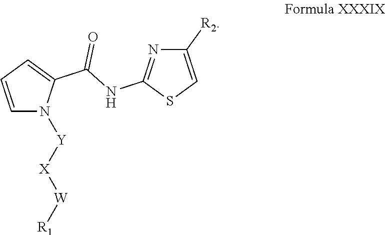

102. The compound, or pharmaceutically acceptable salt thereof, of embodiment 99, wherein the compound has the structure of Formula XXXX:

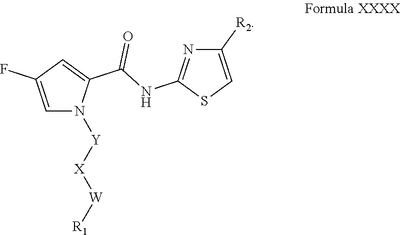

103. The compound, or pharmaceutically acceptable salt thereof, of any one of embodiments 1, 2, or 97 to 99, wherein the compound has the structure of Formula XXXXII:

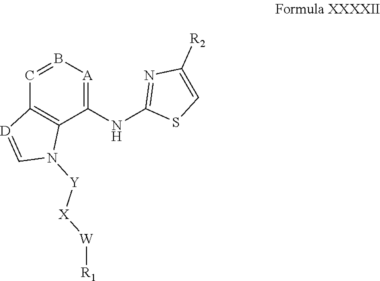

wherein A, B, C, and D are each, independently, CH, CR₄, or N; and
each R₄ is independently optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol.

104. The compound, or pharmaceutically acceptable salt thereof, of embodiment 103, wherein the compound has the structure of Formula XXXXIII:

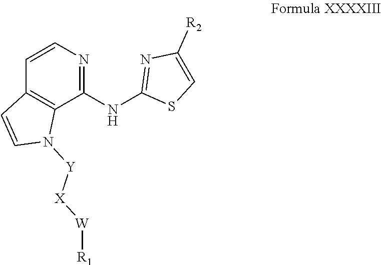

105. The compound, or pharmaceutically acceptable salt thereof, of any one of embodiments 18 or 27 to 104, wherein X is C₁-C₆ alkylene.

106. The compound, or pharmaceutically acceptable salt thereof, of any one of embodiments 18 or 27 to 105, wherein Y is O or NR₃.

107. The compound, or pharmaceutically acceptable salt thereof, of embodiment 106, wherein Y is O.

108. The compound, or pharmaceutically acceptable salt thereof, of embodiment 106, wherein Y is NR₃.

109. The compound, or pharmaceutically acceptable salt thereof, of embodiment 106, wherein Y is NH.

110. The compound, or pharmaceutically acceptable salt thereof, of any one of embodiments 18 or 27 to 109, wherein W is S, O, or NR₃

111. The compound, or pharmaceutically acceptable salt thereof, of embodiment 110, wherein W is S.

112. The compound, or pharmaceutically acceptable salt thereof, of embodiment 110, wherein W is O.

113. The compound, or pharmaceutically acceptable salt thereof, of embodiment 110, wherein W is NR₃.

114. The compound, or pharmaceutically acceptable salt thereof, of embodiment 113, wherein W is NH.

115. The compound, or pharmaceutically acceptable salt thereof, of any one of embodiments 1 to 114, wherein R₂ is an optionally substituted C₁-C₆ alkyl group.

116. The compound, or pharmaceutically acceptable salt thereof, of embodiment 115, wherein R₂ is

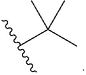 [SMILES: *C(C)(C)C]

117. The compound, or pharmaceutically acceptable salt thereof, of any one of embodiments 1 to 114, wherein R₂ is optionally substituted C₂-C₉ heterocyclyl, such as an optionally substituted pyrrolidine.

118. The compound, or pharmaceutically acceptable salt thereof, of embodiment 115, wherein R₂ is optionally substituted with a halogen, e.g., a fluorine.

119. The compound, or pharmaceutically acceptable salt thereof, of embodiment 117, wherein R₂ is

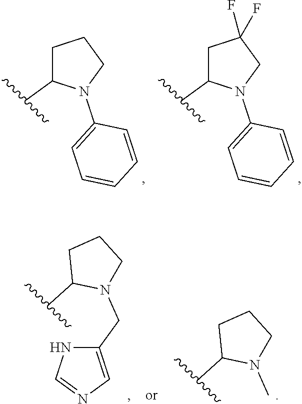

120. The compound, or pharmaceutically acceptable salt thereof, of any one of embodiments 1 to 114, wherein R₂ is an optionally substituted C₂-C₉ heteroaryl, such as an optionally substituted pyridine or optionally substituted oxazole.

121. The compound, or pharmaceutically acceptable salt thereof, of embodiment 120, wherein R₂ is

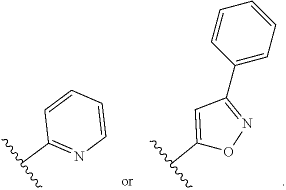 [SMILES: *c1ccccn1 ; *c1cc(-c2ccccc2)no1]

122. The compound, or pharmaceutically acceptable salt thereof, of any one of embodiments 1 to 114, wherein R₂ has the structure X₁—O—Y₁.

123. The compound, or pharmaceutically acceptable salt thereof, of embodiment 122, wherein X₁ is optionally substituted with one or two methyl groups.

124. The compound, or pharmaceutically acceptable salt thereof, of embodiment 122 or 123, wherein Y₁ is optionally substituted phenyl or optionally substituted cyclopropyl.

125. The compound, or pharmaceutically acceptable salt thereof, of any one of embodiments 122 to 123,
wherein R₂ is, or

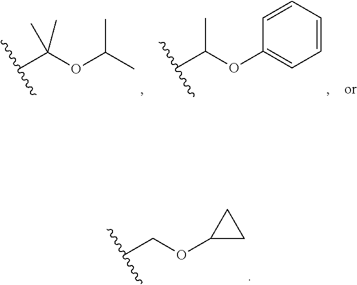

126. The compound, or pharmaceutically acceptable salt thereof, of any one of embodiments 1 to 114, wherein R₂ is an optionally substituted C₆-C₁₀ aryl.

127. The compound, or pharmaceutically acceptable salt thereof, of embodiment 126, wherein R₂ is

phenyl,

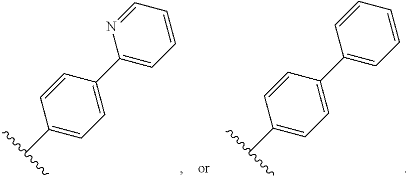 [SMILES: *c1ccc(-c2ccccn2)cc1 ; *c1ccc(-c2ccccc2)cc1]

128. The compound, or pharmaceutically acceptable salt thereof, of any one of embodiments 1 to 114, wherein R² is an optionally substituted C₂-C₆ alkenyl.

129. The compound, or pharmaceutically acceptable salt thereof, of embodiment 128, wherein R² is

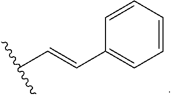 [SMILES: */C=C/c1ccccc1]

130. The compound, or pharmaceutically acceptable salt thereof, of any one of embodiments 1 to 129, wherein R₁ is an optionally substituted C₂-C₉ heteroaryl.

131. The compound, or pharmaceutically acceptable salt thereof, of embodiment 130, wherein R₁ is

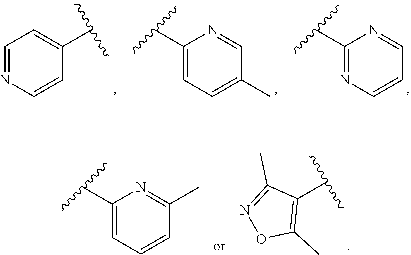

132. The compound, or pharmaceutically acceptable salt thereof, of any one of embodiments 1 to 129, wherein R₁ is Rₐ-R_{b}-R_{c}.

133. The compound, or pharmaceutically acceptable salt thereof, of embodiment 132, wherein Rₐ is an optionally substituted C₆-C₁₀ arylene.

134. The compound, or pharmaceutically acceptable salt thereof, of embodiment 132 wherein R_{c} is an optionally substituted C₂-C₉ heteroarylene.

135. The compound, or pharmaceutically acceptable salt thereof, of any one of embodiments 132 to 134, wherein R_{b} is —O—.

136. The compound, or pharmaceutically acceptable salt thereof, of any one of embodiments 132, 133, or 135, wherein R₁ is

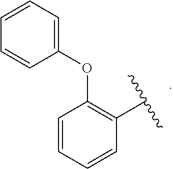 [SMILES: *c1ccccc1Oc1ccccc1]

137. The compound, or pharmaceutically acceptable salt thereof, of any one of embodiments 132 to 134, wherein R_{b} is an optionally substituted C₁-C₆ alkylene.

138. The compound, or pharmaceutically acceptable salt thereof, of any one of embodiments 132, 133, or 137, wherein R_{b} is

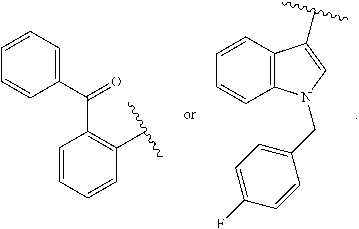 [SMILES: *c1ccccc1C(=O)c1ccccc1 ; *c1cn(Cc2ccc(F)cc2)c2ccccc12]

139. The compound, or pharmaceutically acceptable salt thereof, of any one of embodiments 1 to 129, wherein R₁ is an optionally substituted optionally substituted C₂-C₉ heteroaryl C₁-C₆ alkyl.

140 The compound, or pharmaceutically acceptable salt thereof, of embodiment 139, wherein R₁ is

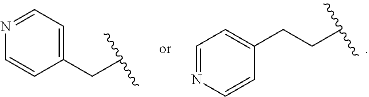 [SMILES: *Cc1ccncc1 ; *CCc1ccncc1]

141. The compound, or pharmaceutically acceptable salt thereof, of any one of embodiments 1 to 129, wherein R₁ is an optionally substituted C₆-C₁₀ aryl.

142. The compound, or pharmaceutically acceptable salt thereof, of embodiment 141, wherein R₁ is

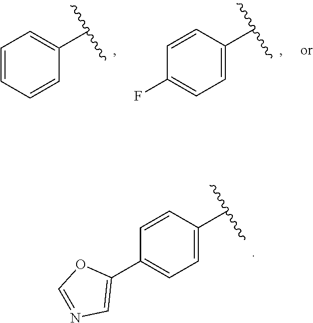 [SMILES: *c1ccccc1 ; *c1ccc(F)cc1 ; *c1ccc(-c2cnco2)cc1]

143. The compound, or pharmaceutically acceptable salt thereof, of any one of embodiments 1 to 142, wherein X is

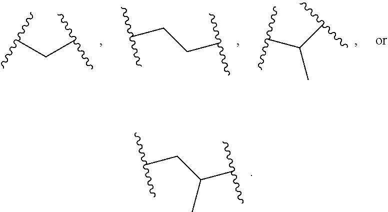

144. The compound, or pharmaceutically acceptable salt thereof, of embodiment 143, wherein X is

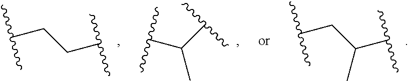

145. A compound having the structure of any one of compounds 1 to 50 in Table 1, or a pharmaceutically acceptable salt thereof.

146. A compound having the structure

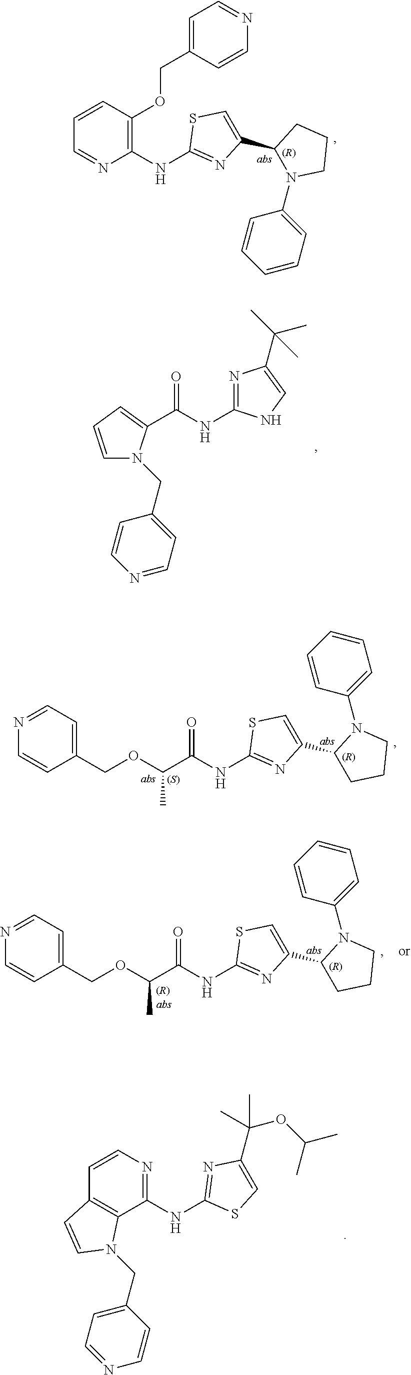

147. A pharmaceutical composition comprising a compound of any one of embodiments 1 to 146, or a pharmaceutically acceptable salt thereof, and a pharmaceutically acceptable excipient.

148. A method of treating a Sec61-associated disease or disorder in a subject in need thereof, the method comprising administering to the subject an effective amount of a compound, or pharmaceutically acceptable salt thereof, of any one of embodiments 1 to 146 or a pharmaceutical composition of embodiment 147.

149. The method of embodiment 148, wherein the disease or disorder is selected from amyloidosis, light chain amyloidosis, autoantibody diseases, chronic kidney disease, fibrosis, neurodegeneration, autoimmune disease, genetically-defined kidney disease, viral disease, influenza, dengue virus, zika virus, hepatitis B virus, hepatitis C virus, SARS-CoV-2, human immunodeficiency virus, malaria, cancer, glioma, myeloma, multiple types of cancer with solid tumors, autoimmune diseases, rheumatoid arthritis, ankylosing spondylitis, celiac disease, multiple sclerosis, atopic dermatitis, Crohn's disease, psoriasis, allergic asthma, autoimmune antibody diseases, myasthenia gravis, neuromyelitis optica, warm antibody hemolytic anemia, prion disease, immune thrombocytopenic purpura, chronic inflammatory demyelinating polyradiculoneuropathy, fibrotic diseases, idiopathic pulmonary fibrosis, endometriosis, nonalcoholic steatohepatitis, neurodegenerative diseases, Alzheimer's disease, Parkinson's disease, Huntington's disease, amyotrophic lateral sclerosis, hypercholesterolemia, Creutzfeldt-Jakob disease, Gerstmann-Strsussler-Scheinker syndrome, high cholesterol, metabolic syndrome, and fatal familial insomnia.

150. The method of embodiment 149, wherein the disease is a viral disease.

151. The method of embodiment 149, wherein the disease is cancer.

152. The method of embodiment 149, wherein the disease is prion disease.

153. The method of embodiment 149, wherein the disorder is light chain amyloidosis.

154. The method of embodiment 149, wherein the disease is autoimmune antibody disease.

155. The method of embodiment 149, wherein the disease is genetically-defined kidney disease.

156. The method of embodiment 149, wherein the disease is malaria.

157. A method of inhibiting the translocation of a target protein through Sec61, the method comprising contacting a cell with an effective amount of a compound of any one of embodiments 1 to 146, or a pharmaceutically acceptable salt thereof, or a pharmaceutical composition of embodiment 147.

158. The method of embodiment 157, wherein inhibition of translocation is selective for a target protein over a non-target protein.

159. A compound, or pharmaceutically acceptable salt thereof, of any one of embodiments 1 to 146 or a pharmaceutical composition of embodiment 147 for use in a treating a Sec61-associated disease or disorder in a subject in need thereof.

160. The compound, or pharmaceutically acceptable salt thereof, or pharmaceutically composition for use according to embodiment 159, wherein the disease or disorder is selected from amyloidosis, light chain amyloidosis, autoantibody diseases, chronic kidney disease, fibrosis, neurodegeneration, autoimmune disease, genetically-defined kidney disease, viral disease, influenza, dengue virus, zika virus, hepatitis B virus, hepatitis C virus, SARS-CoV-2, human immunodeficiency virus, malaria, cancer, glioma, myeloma, multiple types of cancer with solid tumors, autoimmune diseases, rheumatoid arthritis, ankylosing spondylitis, celiac disease, multiple sclerosis, atopic dermatitis, Crohn's disease, psoriasis, allergic asthma, autoimmune antibody diseases, myasthenia gravis, neuromyelitis optica, warm antibody hemolytic anemia, prion disease, immune thrombocytopenic purpura, chronic inflammatory demyelinating polyradiculoneuropathy, fibrotic diseases, idiopathic pulmonary fibrosis, endometriosis, nonalcoholic steatohepatitis, neurodegenerative diseases, Alzheimer's disease, Parkinson's disease, Huntington's disease, amyotrophic lateral sclerosis, hypercholesterolemia, Creutzfeldt-Jakob disease, Gerstmann-Strsussler-Scheinker syndrome, high cholesterol, metabolic syndrome, and fatal familial insomnia.

161. The compound, or pharmaceutically acceptable salt thereof, or pharmaceutically composition for use according to embodiment 160, wherein the disease is a viral disease.

162. The compound, or pharmaceutically acceptable salt thereof, or pharmaceutically composition for use according to embodiment 160, wherein the disease is cancer.

163. The compound, or pharmaceutically acceptable salt thereof, or pharmaceutically composition for use according to embodiment 160, wherein the disease is prion disease.

164. The compound, or pharmaceutically acceptable salt thereof, or pharmaceutically composition for use according to embodiment 160, wherein the disorder is light chain amyloidosis.

165. The compound, or pharmaceutically acceptable salt thereof, or pharmaceutically composition for use according to embodiment 160, wherein the disease is autoimmune antibody disease.

166. The compound, or pharmaceutically acceptable salt thereof, or pharmaceutically composition for use according to embodiment 160, wherein the disease is genetically-defined kidney disease.

167. The compound, or pharmaceutically acceptable salt thereof, or pharmaceutically composition for use according to embodiment 160, wherein the disease is malaria.

168. A compound of any one of embodiments 1 to 146, or a pharmaceutically acceptable salt thereof, or a pharmaceutical composition of embodiment 147, for use in inhibiting the translocation of a target protein through Sec61.

169. The compound, or pharmaceutically acceptable salt thereof, or pharmaceutically composition for use according to embodiment 166, wherein inhibition of translocation is selective for a target protein over a non-target protein.

### Other Embodiments

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

All references, patents, patent application publications, and patent applications recited herein ae hereby incorporated by reference in their entirety.

Other embodiments are in the claims.

1. A compound, or pharmaceutically acceptable salt thereof, having the structure of Formula 1: 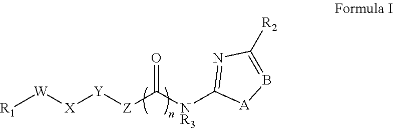
wherein
R₁ is H, optionally substituted C₁-C₆ alkyl, optionally substituted C₃-C₇ cycloalkyl, optionally substituted C₂-C₉ heterocycle, optionally substituted C₆-C₁₀ aryl, optionally substituted C₂-C₉ heteroaryl, C₂-C₉ heteroaryl C₁-C₆ alkyl, or Rₐ-R_{b}-R_{c},
wherein Rₐ is optionally substituted C₆-C₁₀ arylene or optionally substituted C₂-C₉ heteroarylene;
R_{b} is —O— or optionally substituted C₁-C₆ alkylene, and
R_{c} is optionally substituted C₆-C₁₀ aryl;
Z is absent, optionally substituted C₃-C₁₀ cycloalkylene, optionally substituted C₂-C₉ heterocyclylene, C₆-C₁₀ arylene, or optionally substituted C₂-C₉ heteroarylene;
Y is absent, O, or NR₃;
X is absent or optionally substituted C₁-C₆ alkylene;
W is absent, S, O or NR₃;
n is 0 or 1;
A is S, O, NH, or CH_{2;}
B is CH or N;
R₂ is optionally substituted C₁₋₆ alkyl, optionally substituted C₂-C₆ alkenyl, optionally substituted C₃₋₇ cycloalkyl, optionally substituted C₂₋₉ heterocycle, optionally substituted C₆-C₁₀ aryl, optionally substituted C₂₋₉ heteroaryl; optionally substituted C₂-C₉ heteroaryl C₁-C₆ alkyl, optionally substituted C₃-C₇ cycloalkyl C₁-C₆ aryl, optionally substituted C₁-C₆ heteroalkyl, cyano, or has the structure X₁—O—Y₁;
wherein X₁ is optionally substituted C₁-C₆ alkylene, and
Y₁ is optionally substituted C₁-C₆ alkyl, optionally substituted C₃-C₈ cycloalkyl, or optionally substituted C₆-C₁₀ aryl; and
each R₃ is, independently, H or C₁-C₆ alkyl.

2. The compound, or pharmaceutically acceptable salt thereof, of claim 1, wherein n is 1.

3. The compound, or pharmaceutically acceptable salt thereof, of claim 1, wherein the compound has the structure of Formula II:

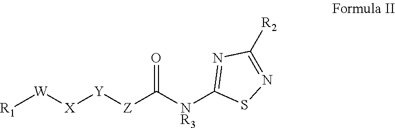

4. The compound, or pharmaceutically acceptable salt thereof, of claim 1, wherein the compound has the structure of Formula III:

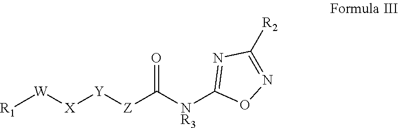

5. The compound, or pharmaceutically acceptable salt thereof, of claim 1, wherein the compound has the structure of Formula IV:

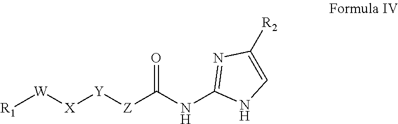

6. The compound, or pharmaceutically acceptable salt thereof, of any one of claims 3 to 5, wherein Z is optionally substituted C₆-C₁₀ aryl, or optionally substituted C₂-C₉ heteroaryl.

7. The compound, or pharmaceutically acceptable salt thereof, of any one of claims 1 to 6, wherein Y is O or NR₃.

8. The compound, or pharmaceutically acceptable salt thereof, of claim 7, wherein Y is O.

9. The compound, or pharmaceutically acceptable salt thereof, of claim 7, wherein Y is NR₃.

10. The compound, or pharmaceutically acceptable salt thereof, of claim 7, wherein Y is NH.

11. The compound, or pharmaceutically acceptable salt thereof, of any one of claims 3 to 10 wherein X is optionally substituted C₁-C₆ alkylene.

12. The compound, or pharmaceutically acceptable salt thereof, of any one of claims 1 to 11, wherein W is S, O or NR₃.

13. The compound, or pharmaceutically acceptable salt thereof, of claim 12, wherein W is S.

14. The compound, or pharmaceutically acceptable salt thereof, of claim 12, wherein W is O.

15. The compound, or pharmaceutically acceptable salt thereof, of claim 12, wherein W is NR₃.

16. The compound, or pharmaceutically acceptable salt thereof, of claim 15, wherein W is NH.

17. The compound, or pharmaceutically acceptable salt thereof, of claim 1 or 2 wherein A is S and B is C.

18. The compound, or pharmaceutically acceptable salt thereof, of any one of claims 1, 2, or 17, wherein the compound has the structure of Formula IV: 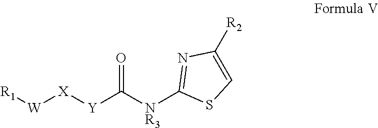
wherein Y is O or NR₃.

19. The compound, or pharmaceutically acceptable salt thereof, of claim 18, wherein X is optionally substituted C₁-C₆ alkylene.

20. The compound, or pharmaceutically acceptable salt thereof, of claim 19, wherein Y is O.

21. The compound, or pharmaceutically acceptable salt thereof, of claim 19, wherein Y is NR₃

22. The compound, or pharmaceutically acceptable salt thereof, of claim 22, wherein Y is NH.

23. The compound, or pharmaceutically acceptable salt thereof, of any one of claims 1, 2, or 17, wherein the compound has the structure of Formula V: 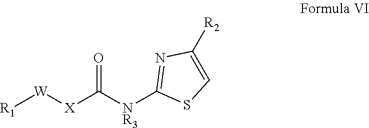
wherein W is NR₃.

24. The compound, or pharmaceutically acceptable salt thereof, of claim 22, wherein X is optionally substituted C₁-C₆ alkylene.

25. The compound, or pharmaceutically acceptable salt thereof, of claim 23 or 24, wherein W is NH.

26. The compound, or pharmaceutically acceptable salt thereof, of claim 1, wherein n is 0.

27. The compound, or pharmaceutically acceptable salt thereof, of claims 1 to 5, 7 to 17, or 26, wherein Z is optionally substituted phenylene, optionally substituted pyridinylene, optionally substituted pyrimidinylene, optionally substituted pyridazinylene, optionally substituted pyrazinylene, optionally substituted triazinylene, optionally substituted tetrazinylene, optionally substituted thiophenylene, optionally substituted pyrrolylene, optionally substituted furanylene, optionally substituted pyrazolylene, optionally substituted thiazolylene, optionally substituted oxadiazolylene, optionally substituted thiadiazolylene, optionally substituted isoxazolylene, optionally substituted isothiazolylene, optionally substituted thiazolylene, optionally substituted oxazolylene, optionally substituted imidazolylene, optionally substituted cyclohexylene, optionally substituted cyclopentylene, optionally substituted cyclobutylene, optionally substituted cyclopropylene, optionally substituted cycloheptylene, optionally substituted cyclooctylene, optionally substituted indolylene, or optionally substituted azaindolylene.

28. The compound, or pharmaceutically acceptable salt thereof, of any one of claims 1 to 17, 26, or 27, wherein Z has the structure of Formula VIi: 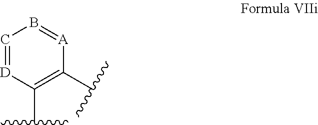
wherein A, B, C and D are, each, independently, N, CH, or CR₄; and
each R₄ is independently optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol.

29. The compound, or pharmaceutically acceptable salt thereof, of claim 28, wherein Z has the structure of Formula VIIi: 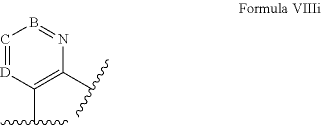
wherein B, C, and D are each, independently, CH or CR₄.

30. The compound, or pharmaceutically acceptable salt thereof, of claim 28, wherein Z has the structure of formula VIIIi: 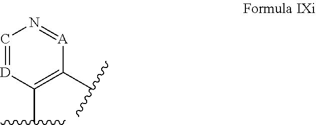
wherein A, C, and D are each, independently, CH or CR₄.

31. The compound, or pharmaceutically acceptable salt thereof, of claim 28, wherein Z has the structure of formula IXi: 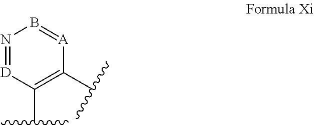
wherein A, B, and D are each, independently, CH or CR₄.

32. The compound, or pharmaceutically acceptable salt thereof, of claim 28, wherein Z has the structure of Formula Xi: 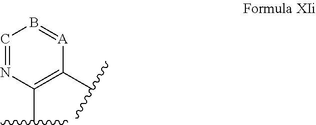
wherein A, B, and C are each, independently, CH or CR₄.

33. The compound, or pharmaceutically acceptable salt thereof, of claim 28, wherein Z has the structure of Formula Xli: 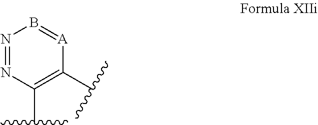
wherein A and B are each, independently, CH or CR₄.

34. The compound, or pharmaceutically acceptable salt thereof, of claim 28, wherein Z has the structure of Formula XIIi: 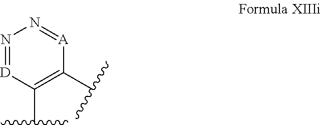
wherein A and D are each, independently, CH or CR₄.

35. The compound, or pharmaceutically acceptable salt thereof, of claim 28, wherein Z has the structure of Formula XIIIi: 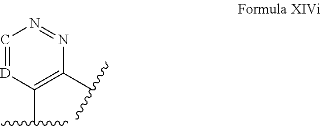
wherein C and D are each, independently, CH or CR₄.

36. The compound, or pharmaceutically acceptable salt thereof, of claim 28, wherein Z has the structure of Formula XIVi: 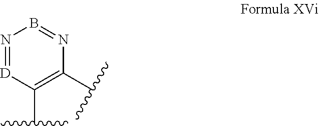
wherein B and D are each, independently, CH or CR₄.

37. The compound, or pharmaceutically acceptable salt thereof, of claim 28, wherein Z has the structure of Formula XVi: 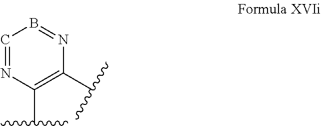
wherein B and C are each, independently, CH or CR₄.

38. The compound, or pharmaceutically acceptable salt thereof, of claim 28, wherein Z has the structure of Formula XVIi: 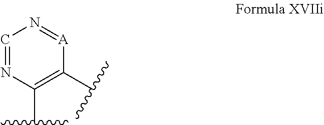
wherein A and C are each, independently, CH or CR₄.

39. The compound, or pharmaceutically acceptable salt thereof, of claim 28, wherein Z has the structure of Formula XVIIi: 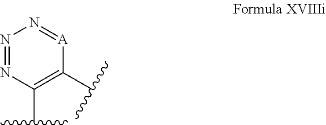
wherein A is CH or CR₄.

40. The compound, or pharmaceutically acceptable salt thereof, of claim 28, wherein Z has the structure of Formula XVIIIi: 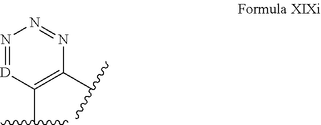
wherein D Is CH or CR₄.

41. The compound, or pharmaceutically acceptable salt thereof, of claim 28, wherein Z has the structure of Formula XIXi: 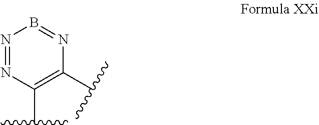
wherein B is CH or CR₄.

42. The compound, or pharmaceutically acceptable salt thereof, of claim 28, wherein Z has the structure of Formula XXi: 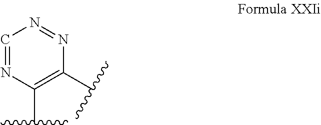
wherein C is CH or CR₄.

43. The compound, or pharmaceutically acceptable salt thereof, of claim 28, wherein Z has the structure of Formula XXIi: 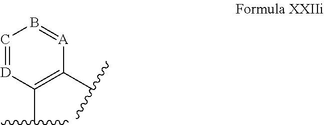
wherein A, B, C, and D are each, independently, CH or CR₄

44. The compound, or pharmaceutically acceptable salt thereof, of any one of claims 1 to 5, 7 to 17, 26, or 27, wherein Z has the structure of Formula XXIIi: 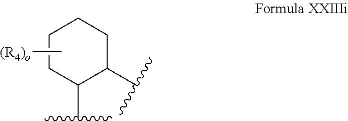
wherein o is 0, 1, 2, 3, or 4; and
each R₄ is, independently, optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol.

45. The compound, or pharmaceutically acceptable salt thereof, of any one of claims 1 to 5, 7 to 17, 26, or 27, wherein Z has the structure of Formula XXIIIi: 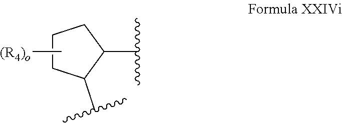
wherein o is 0, 1, 2, or 3; and
each R₄ is, independently, optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol.

46. The compound, or pharmaceutically acceptable salt thereof, of any one of claims 1 to 17, 26, or 27, wherein Z has the structure of Formula XXIVi: 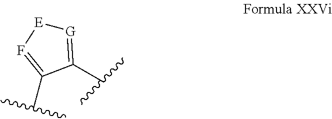
wherein E is NH, O, or S;
F and G are each, independently, N, CH, or CR₄; and
each R₄ is independently optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol.

47. The compound, or pharmaceutically acceptable salt thereof, of claim 46, wherein Z has the structure of Formula XXVi: 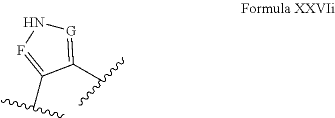
wherein F and G are each, independently, CH or CR₄.

48. The compound, or pharmaceutically acceptable salt thereof, of claim 46, wherein Z has the structure of Formula XXVIi: 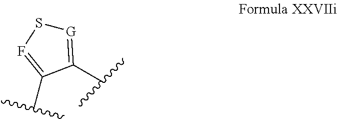
wherein F and G are each, independently, CH or CR₄

49. The compound, or pharmaceutically acceptable salt thereof, of claim 46, wherein Z has the structure of Formula XXVIIi: 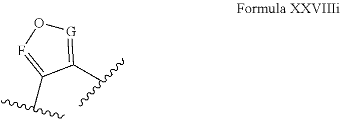
wherein F and G are each, independently, CH or CR₄.

50. The compound, or pharmaceutically acceptable salt thereof, of any one of claims 1 to 17, 26, or 27, wherein Z has the structure of Formula XXVIIIi: 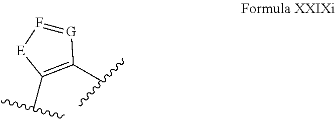
wherein E is NH, O, or S;
F and G are each, independently, N, CH, or CR₄; and
each R₄ is independently optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol.

51. The compound, or pharmaceutically acceptable salt thereof, of claim 50, wherein Z has the structure of Formula XXIXi: 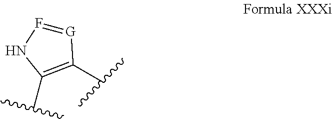
wherein F and G are each, independently, CH or CR₄.

52. The compound, or pharmaceutically acceptable salt thereof, of claim 50, wherein Z has the structure of Formula XXXi: 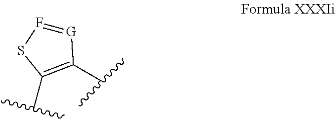
wherein F and G are each, independently, CH or CR₄.

53. The compound, or pharmaceutically acceptable salt thereof, of claim 50, wherein Z has the structure of Formula XXXIi: 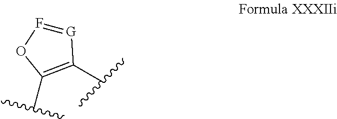
wherein F and G are each, independently, CH or CR₄.

54. The compound, or pharmaceutically acceptable salt thereof, of any one of claims 1 to 17, 26, or 27, wherein Z has the structure of Formula XXXIIi: 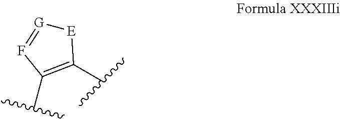
wherein E is NH, O, or S;
F and G are each, independently, N, CH, or CR₄; and
each R₄ is independently optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol.

55. The compound, or pharmaceutically acceptable salt thereof, of claim 54, wherein Z has the structure of Formula XXXIIIi: 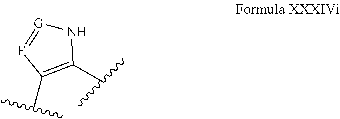
wherein F and G are each, independently, CH or CR₄.

56. The compound, or pharmaceutically acceptable salt thereof, of claim 54, wherein Z has the structure of Formula XXXIVi: 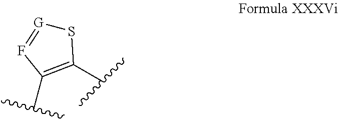
wherein F and G are each, independently, CH or CR₄.

57. The compound, or pharmaceutically acceptable salt thereof, of claim 54, wherein Z has the structure of Formula XXXVi: 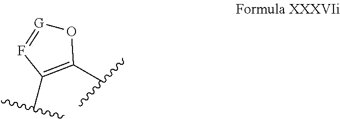
wherein F and G are each, independently, CH or CR₄.

58. The compound, or pharmaceutically acceptable salt thereof, of any one of claims 1 to 17, 26, or 27, wherein Z has the structure of Formula XXXVIi: 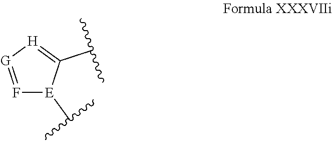
wherein E is N or CH;
F, G, and H are each, independently, N, CH, or CR₄; and
each R₄ is independently optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol.

59. The compound, or pharmaceutically acceptable salt thereof, of claim 58, wherein Z has the structure of Formula XXXVIIi:

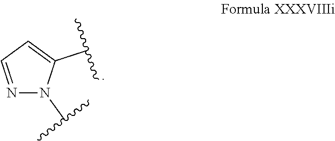 [SMILES: *c1ccnn1*]

60. The compound, or pharmaceutically acceptable salt thereof, of claim 58, wherein Z has the structure of Formula XXXVIIii:

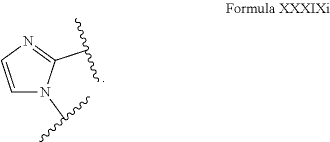 [SMILES: *c1nccn1*]

60. The compound, or pharmaceutically acceptable salt thereof, of claim 58, wherein Z has the structure of Formula XXXVIIIi:

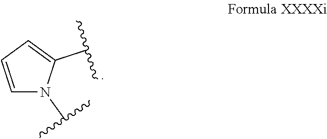 [SMILES: *c1cccn1*]

61. The compound, or pharmaceutically acceptable salt thereof, of claim 58, wherein Z has the structure of Formula XXXIXi:

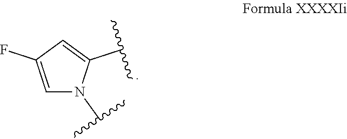 [SMILES: *c1cc(F)cn1*]

62. The compound, or pharmaceutically acceptable salt thereof, of any one of claims 1 to 17, 26, or 27, wherein Z has the structure of Formula XXXXIIi: 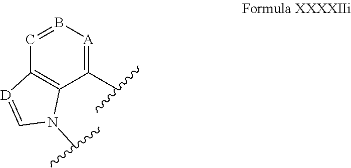
wherein A, B, C, and D are each, independently, CH, CR₄, or N; and
each R₄ is independently optionally substituted aryl, optionally substituted carbocyclyl, halogen, hydroxyl, optionally substituted heteroalkyl, optionally substituted heteroaryl, optionally substituted heterocyclyl, optionally substituted amino azido, cyano, nitro, or thiol.

63. The compound, or pharmaceutically acceptable salt thereof, of claim 62, wherein Z has the structure of Formula XXXX:

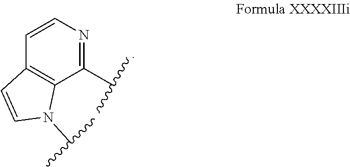 [SMILES: *c1nccc2ccn(*)c12]

64. The compound, or pharmaceutically acceptable salt thereof, of any one of claims 17 or 26 to 63 wherein X is C₁-C₆ alkylene.

65. The compound, or pharmaceutically acceptable salt thereof, of any one of claims 17 or 26 to 64, wherein Y is O or NR₃.

66. The compound, or pharmaceutically acceptable salt thereof, of claim 64, wherein Y is O.

67. The compound, or pharmaceutically acceptable salt thereof, of claim 64, wherein Y is NR₃.

68. The compound, or pharmaceutically acceptable salt thereof, of claim 66, wherein Y is NH.

69. The compound, or pharmaceutically acceptable salt thereof, of any one of claims 17 or 26 to 68, wherein W is S, O, or NR₃.

70. The compound, or pharmaceutically acceptable salt thereof, of claim 69, wherein W is S.

71. The compound, or pharmaceutically acceptable salt thereof, of claim 69, wherein W is O.

72. The compound, or pharmaceutically acceptable salt thereof, of claim 69, wherein W is NR₃.

73. The compound, or pharmaceutically acceptable salt thereof, of claim 69, wherein W is NH.

74. The compound, or pharmaceutically acceptable salt thereof, of any one of claims 1 to 73, wherein R₂ is optionally substituted C₁-C₆ alkyl.

75. The compound, or pharmaceutically acceptable salt thereof, of claim 74, wherein R₂ is

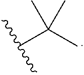 [SMILES: *C(C)(C)C]

76. The compound, or pharmaceutically acceptable salt thereof, of any one of claims 1 to 73, wherein R₂ is optionally substituted C₂-C₉ heterocyclyl.

77. The compound, or pharmaceutically acceptable salt thereof, of claim 73, wherein R₂

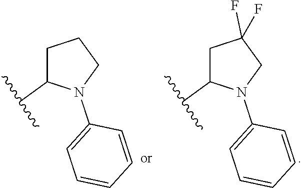 [SMILES: *C1CCCN1c1ccccc1 ; *C1CC(F)(F)CN1c1ccccc1]

78. The compound, or pharmaceutically acceptable salt thereof, of any one of claims 1 to 73, wherein R₂ is optionally substituted C₂-C₉ heteroaryl.

79. The compound, or pharmaceutically acceptable salt thereof, of claim 78, wherein R₂ is

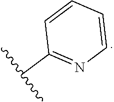 [SMILES: *c1ccccn1]

80. The compound, or pharmaceutically acceptable salt thereof, of any one of claims 1 to 73, wherein R₂ has the structure X₁—O—Y₁.

81. The compound, or pharmaceutically acceptable salt thereof, of claim 80, wherein X₁ is optionally substituted with one or two methyl groups.

82. The compound, or pharmaceutically acceptable salt thereof, of claim 80 or 81, wherein Y₁ is optionally substituted phenyl or optionally substituted cyclopropyl.

83. The compound, or pharmaceutically acceptable salt thereof, of claim any one of claims 80 to 82, wherein R₂ is

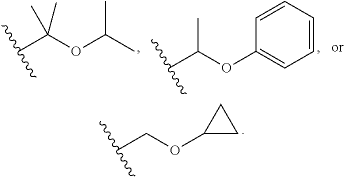

84. The compound, or pharmaceutically acceptable salt thereof, of any one of claims 1 to 73, wherein R₂ is optionally substituted C₆-C₁₀ aryl.

85. The compound, or pharmaceutically acceptable salt thereof, of claim 84, wherein R₂ is phenyl.

86. The compound, or pharmaceutically acceptable salt thereof, of any one of claims 1 to 85, wherein R₁ is optionally substituted C₂-C₉ heteroaryl.

87. The compound, or pharmaceutically acceptable salt thereof, of claim 86, wherein R₁ is

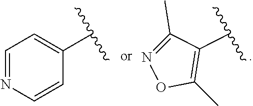

88. The compound, or pharmaceutically acceptable salt thereof, of any one of claims 1 to 85, wherein R₁ is Rₐ-R_{b}-R_{c}.

89. The compound, or pharmaceutically acceptable salt thereof, of claim 88, wherein R_{b} is —O—.

90. The compound, or pharmaceutically acceptable salt thereof, of claim 88 or 89, wherein R₁ is

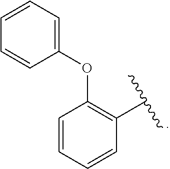 [SMILES: *c1ccccc1Oc1ccccc1]

91. The compound, or pharmaceutically acceptable salt thereof, of claim 88, wherein R_{b} is optionally substituted C₁-C₆ alkylene.

92. The compound, or pharmaceutically acceptable salt thereof, of claim 88 or 91, wherein R₁ is

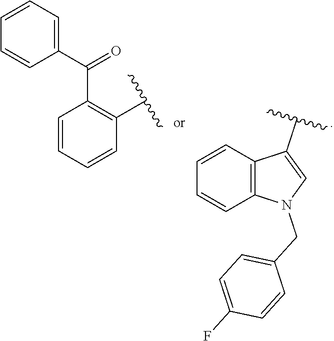 [SMILES: *c1ccccc1C(=O)c1ccccc1 ; *c1cn(Cc2ccc(F)cc2)c2ccccc12]

93. The compound, or pharmaceutically salt thereof, of any one of claims 1 to 85, wherein R₁ is an optionally substituted C₂-C₉ heteroaryl C₁-C₆ alkyl.

94. The compound, or pharmaceutically acceptable salt thereof, of claim 93, wherein R₁ is or

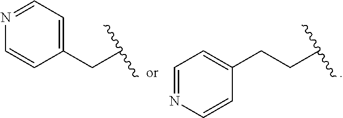 [SMILES: *Cc1ccncc1 ; *CCc1ccncc1]

95. The compound, or pharmaceutically acceptable salt thereof, of any one of claims 1 to 85, wherein R₁ is an optionally substituted C₆-C₁₀ aryl.

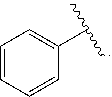 [SMILES: *c1ccccc1]

96. The compound, or pharmaceutically acceptable salt thereof, of claim 95, wherein R₁ is.

97. The compound, or pharmaceutically acceptable salt thereof, of any one of claims 1 to 96, wherein X is

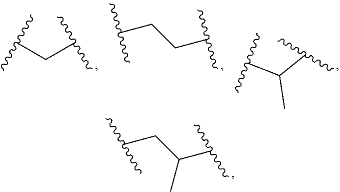

98. A compound having the structure of any one of compounds 1 to 50 in Table 1, or a pharmaceutically acceptable salt thereof.

99. A pharmaceutical composition comprising a compound of any one of claims 1 to 98, or a pharmaceutically acceptable salt thereof, and a pharmaceutically acceptable excipient.

100. A method of treating a Sec61-associated disease or disorder in a subject in need thereof, the method comprising administering to the subject an effective amount of a compound, or pharmaceutically acceptable salt thereof, of any one of claims 1 to 98 or a pharmaceutical composition of claim 99.

101. The method of claim 100, wherein the disease or disorder is selected from amyloidosis, light chain amyloidosis, autoantibody diseases, chronic kidney disease, fibrosis, neurodegeneration, autoimmune disease, genetically-defined kidney disease, viral disease, influenza, dengue virus, zika virus, hepatitis B virus, hepatitis C virus, SARS-CoV-2, human immunodeficiency virus, malaria, cancer, glioma, myeloma, multiple types of cancer with solid tumors, autoimmune diseases, rheumatoid arthritis, ankylosing spondylitis, celiac disease, multiple sclerosis, atopic dermatitis, Crohn's disease, psoriasis, allergic asthma, autoimmune antibody diseases, myasthenia gravis, neuromyelitis optica, warm antibody hemolytic anemia, prion disease, immune thrombocytopenic purpura, chronic inflammatory demyelinating polyradiculoneuropathy, fibrotic diseases, idiopathic pulmonary fibrosis, endometriosis, nonalcoholic steatohepatitis, neurodegenerative diseases, Alzheimer's disease, Parkinson's disease, Huntington's disease, amyotrophic lateral sclerosis, hypercholesterolemia, Creutzfeldt-Jakob disease, Gerstmann-Strsussler-Scheinker syndrome, high cholesterol, metabolic syndrome, and fatal familial insomnia.

102. The method of claim 101, wherein the disease is viral diseases.

103. The method of claim 101, wherein the disease is cancer.

104. The method of claim 101, wherein the disease is prion disease.

105. The method of claim 101, wherein the disorder is light chain amyloidosis.

106. The method of claim 101, wherein the disease is autoimmune antibody disease.

107. The method of claim 101, wherein the disease is genetically-defined kidney disease.

108. The method of claim 101, wherein the disease is malaria.

109. A method of inhibiting the translocation of a target protein through Sec61, the method comprising contacting a cell with an effective amount of a compound of any one of claims 1 to 98, or a pharmaceutically acceptable salt thereof, or a pharmaceutical composition of claim 99.

110. The method of claim 109, wherein inhibition of translocation is selective for a target protein over a non-target protein.


SEQUENCE LISTING (4 sequences)

SEQ ID NO: 1 - protein, 200 aa, synthetic construct
MANLGCWMLVLFVATWSDLGLCKKRPKPGGWNKPTENNEDFNIVAVASNFATTDLDADRGKLPGKKLPLEVLKEMEANARKAGCTRGCLICLSHIKCTPKMKKFIPGRCHTYEGDKESAQGGIGEAIVDIPEIPGFKDLEPMEQFIAQVDLCVDCTTGCLKGLANVQCSDLLKKWLPQRCATFASKIQGQVDKIKGAGGD

SEQ ID NO: 2 - protein, 30 aa, synthetic construct
MANLGCWMLVLFVATWSDLGLCKKRPKPGG

SEQ ID NO: 3 - protein, 33 aa, synthetic construct
MQIPQAPWPVVWAVLQLGWRPGWPLDSPDRPWN

SEQ ID NO: 4 - protein, 31 aa, synthetic construct
MANLGCWMLVFVATWSDLGLCKKRPKPGGWN